(19) United States
(12) Patent Application Publication
(10) Pub. No.: US 2026/0090757 A1
(43) Pub. Date: Apr. 2, 2026

(54) WEARABLE ELECTROCARDIOGRAM (ECG) MONITORING SYSTEMS AND AUTOMATIC ANTI-ARRHYTHMIA SYSTEMS

(71) Applicant: David C. Shaw, Torrance, CA (US)

(72) Inventor: David C. Shaw, Torrance, CA (US)

(21) Appl. No.: 19/332,747

(22) Filed: Sep. 18, 2025

Related U.S. Application Data
(60) Provisional application No. 63/732,838, filed on Sep. 27, 2024.
(60) Provisional application No. 63/833,068, filed on Oct. 21, 2024.

(51) Int. Cl. A61B 5/363; A61B 5/256

(52) U.S. Cl. CPC A61B 5/363; A61B 5/256

(57) ABSTRACT

Wearable electrocardiogram (ECG) systems configured to record ECG from ears (auricular ECG systems), fingers (ear-finger or finger-finger ECG systems) or toes, comprising a processing unit configured to analyze the ECG data to detect serious cardiac arrhythmias or impending serious cardiac arrhythmia. Automatic anti-arrhythmia systems comprise one of the ECG systems and a transcutaneous auricular vagus nerve stimulation (taVNS) unit. When serious cardiac arrythmias are detected, the anti-arrhythmia systems may automatically send pre-determined electric stimuli to a vagus nerve to alleviate or stop the cardiac arrhythmias. The ECG electrodes and the stimulating electrode of the taVNS unit are located on a surface of a housing structure made of flexible elastic and adaptable material such that these electrodes will be naturally and snugly contacting the wearer's skin. These wearable ECG and anti-arrhythmia systems are suitable for easy self-installation, self-removal and daily ambulatory use.

### CROSS REFERENCE TO RELATED APPLICATIONS

This application claims priority to and the benefit of the filing date of U.S. Provisional Application No. 63/732,838, filed on Sep. 27, 2024, entitled “Automatic Anti-arrhythmia Device”, which is hereby incorporated by reference in its entirety. This application also claims priority to and the benefit of the filing date of U.S. Provisional Application No. 63/833,068, filed on Oct. 21, 2024, entitled “Auricular Electrocardiography Automatic Anti-arrhythmia System”, which is hereby incorporated by reference in its entirety.

### FIELD OF THE INVENTION

This patent specification relates to the field of electrocardiogram (ECG or EKG), automatic ECG analysis, and cardiac arrhythmia. More specifically, the present invention relates to the field of wearable ECG recorder systems and transcutaneous auricular vagus nerve stimulation.

### BACKGROUND

The standard 12-lead electrocardiogram (ECG or EKG) includes ten electrodes placed on the patient's limbs and on the surface of chest skin. The 10 electrodes are attached as follows: 1 on each wrist and left ankle and 6 across the precordium. A pair of two electrodes forms a lead. There are two types of leads. One of them is the bipolar leads that measure the potential difference between two electrodes (or between two specific locations). Another type is unipolar leads that measure the electrical potential at a single point relative to a central terminal (reference point or a virtual electrode). The most commonly used virtual electrode is called Wilson's central terminal which is the average potential of 3 limb electrodes attached to the right arm, left arm and left foot. The ECG is essentially a graph of voltage versus time of the electrical activities of the heart. The ECG machine measures the difference of electric potentials between two locations of the electrode attachments. Thus, a pair of electrodes (or a pair of an electrode and a virtual electrode) is the minimal requirement to do a single-lead ECG.

There are significant advances in electrocardiography development of dry electrodes and even smaller devices and electronics to be included in smart watches and health trackers (or called fitness trackers). Because of the small size, these smart watches or health trackers usually only have two electrodes to form a single lead for ECG. The small size and being wearable on the wrist provide major advantages. However, the currently available smart watches have a disadvantage that they cannot automatically record ECG. They usually require action from the wearer (to use a finger from the opposite hand to touch Digital Crown for Apple Watch or Home Button for Samsung Galaxy Watch) in order to start the ECG recording. They can record ECG only for a short duration. Sophisticated automatic interpretation algorithms have also been developed for most modern ECG machines. These algorithms can analyze the ECG data and calculate ECG features such as PR interval, QT interval and QRS duration etc. They can also detect various cardiac arrhythmias, including atrial fibrillation (AFib or AF), atrial flutter (AFL), supraventricular tachycardia (SVT), ventricular tachycardia (VT), ventricular fibrillation (VF) and Wolff-Parkinson-White (WPW) syndrome etc. Most smart watches with ECG capability also have automatic ECG interpretation algorithms.

Conventional ECGs are recorded for a very short time (for example 1-2 minutes). Continuous ECG monitoring, like Holter monitor and Zio Patch monitor, can monitor the heart significantly longer. Most Holter monitors employ between three and eight electrodes, separated from each other. A newer technology, like Zio Patch (or Zio XT Patch), uses a single patch which contains two separated electrodes, forming a single bipolar lead. Both Holter monitor and Zio Patch monitor use special glue or attachment material to attach the electrodes to the chest skin. It is difficult to keep the electrodes attached for a long time. Patients often have skin reactions to the attachment material. Holter monitor is usually used for 24-48 hours. Zio Patch can be used for up to 14 days. None of them can be used for extended monitoring for months or even years.

With technological advancement, dry electrodes, wireless dry electrodes, and miniaturization trend, very tiny electrodes (sensor electrodes) accompanied with sophisticated algorithms have been developed in recent years. These miniature sensor electrodes are used in various smartwatches and health trackers. There are some smart watches or wearable health trackers that can do ECG. However, they cannot automatically do ECG on a continuous basis. Apple Watches (series 4-10) has two electrodes. One electrode is built on the undersurface of the Apple Watch and the electrode contacts the wearer's wrist skin. The other electrode is located at its Digital Crown. The wearer needs to use a finger from the other hand to touch the Digital Crown for the Apple Watch to start ECG recording. Samsung Galaxy Watch (4-8) also has two electrodes. One electrode is built on the undersurface of the Samsung Watch and the electrode contacts the wearer's wrist skin. The other electrode is built on its Home Button. The wearer needs to use a finger from the other hand to touch the Home Button for the Samsung Watch to start ECG recording. Because of the need for the wearer's action (to put a finger from the other hand onto a certain part of the watches), these smart watches cannot monitor the heart on a continuous basis. For example, Apple Watch usually does each ECG for 30 seconds. Cardiac arrhythmia (for example paroxysmal atrial fibrillation) can occur suddenly and might not have obvious symptoms. Thus, the wearers might not be aware of the presence of arrhythmia and very likely would miss the opportunity to turn on the ECG recording.

Modern technology has made doing ECG monitoring on upper arm become feasible. In 2019 Agel Villegas et al reported successfully doing bipolar ECG monitoring for long-term (72 hours) by placing two electrodes on left upper arm, close to the axilla area. More arm-based wearable ECG designs were published since then. In 2022, Omar Escalona et al (Ulster University, UK) reported wearable ECG by using MUAC (mid-upper arm circumference) armband on left upper arm to do continuous ECG monitoring. However, using wearable arm-based ECG for monitoring purpose often have to overcome the disadvantage of too much muscle artifacts and movement artifacts. With further advancement of technology, including wireless electrodes, advanced signal processing techniques and algorithms, recording ECG from an ear or both ears has become feasible,

Because of the above reasons there exists a great need to have a novel wearable ECG monitoring device which can be used for very long time (months or years), and can record ECG on continuous basis for monitoring of various cardiac arrhythmias.

Cardiac arrhythmia is a very common medical problem in the USA and in the world. There are several types of cardiac arrhythmia. Some of the arrhythmias could be quite serious, including atrial fibrillation (AFib or AF), atrial flutter (AFL), supraventricular tachycardia (SVT), ventricular tachycardia (VT), ventricular fibrillation (VF) and Wolff-Parkinson-White (WPW) syndrome. Ventricular fibrillation is one of the major causes of sudden cardiac death. AFib is also very prevalent and affects millions of people in the USA. AFib is one of the major causes of stroke. Prolonged AFib could lead to heart failure and other cardiac complications. Symptoms of AFib could be subtle and some patients might not be aware of it. The first time they know about the presence of AFib might be after being sent to an emergency room with a stroke and then found to have AFib. AFib could be permanent or paroxysmal. The paroxysmal AFib could start anytime of the day and could start during sleep. Ideally, therapeutic intervention for AFib, AFL, SVT, VT, VF and WPW should be started as soon as possible. However, many patients with these cardiac arrhythmias often have significant delay before they receive therapeutic intervention. The delay could be due to various factors, for example, patients being unaware of it, arrhythmia occurred during sleep, ambulance not available immediately, or emergency rooms not close-by, or healthcare provider needing preparation time, etc.

Sometimes healthcare providers might teach some patients to use self-administered maneuvers to stimulate the vagus nerve for AFib or SVT. However, there are many disadvantages with this approach. These self-administered non-electrical vagus nerve stimulations are not very effective. Many patients are unable to do these maneuvers correctly. Sometimes the patients might misjudge the situation and incorrectly use the maneuvers for the wrong situation. Not infrequently, the patients may not be aware of the presence of AFib or SVT and fail to use these maneuvers.

Notification function is valuable. There are various types of smart watches (for example Apple Watches, Series 4-10 or Ultra or Ultra-2 and Samsung Galaxy Watch 4-8) that can take electrocardiogram (ECG or EKG) and can notify the wearer when atrial fibrillation or tachycardia are detected. Some wearable health trackers could also have similar notification functions. The Apple Watch can detect the sinus rhythm classification with 99.6% accuracy, and reach the AFib classification with 98.3% accuracy. With the high accuracy, these watches are cleared by US Food and Drug Administration (FDA). These smart watches or wearable health trackers help to identify the presence of serious cardiac arrhythmia. However, these smart watches or health trackers often require the wearers to initiate the ECG recording and would not help if the wearers are not aware of the presence of the arrhythmia. Most of them can only do on-demand snapshot ECG recording for a very short time (for example, for 30 seconds). They cannot do continuous ECG monitoring.

In 2013, a company (AliveCor's KardiaMobile) developed an ECG system with the ECG electrodes based on finger(s). Users would place their fingers on the ECG electrodes to record an ECG, which would then be transmitted to the user's smartphone or tablet via the Kardia app. Around 2016, Beurer developed a device Beurer ME 90 mobile ECG device that can record ECG through chest-arm-hand or hand-arm-upper body-arm-hand contact. In 2014, MyDiagnostick was developed and it can record a single-lead ECG by having the user hold the device with both hands for one minute. The device uses embedded dry electrodes in the handles to detect the electrical activity of the heart. The device analyzes the R-R interval irregularity to detect potential atrial fibrillation (AFib). More recently, smart rings with ECG capability have been developed, for example V11 ECG Smart Ring uses electrodes placed against the finger skin to detect the electrical signals generated by the heart. In 2021, Circular developed a smart ring, Circul+, with that a user can record ECG by placing a finger from opposite hand onto a specific section on the ring, which completes the circuit and allows the ECG to be measured. In 2025, Circular Ring Gen 2 was announced with ring on finger and the ring includes ECG capability with its FDA-cleared AFib detection algorithm. It's made of titanium, boasts an eight-day battery life, and has enhanced sensors for more precise readings. However, none of these devices can start therapeutic intervention automatically. Thus, there exists a great need to automatically start therapeutic intervention immediately when certain serious cardiac arrhythmia is detected.

There are implantable (invasive) chest-based ECG monitors which require a surgery for placement. For example, Abbott developed Assert-IQ ICM which is an insertable cardiac monitor, usually placed subcutaneously in the left anterior chest. They use enhanced algorithms to reduce false detection of AFib by 98.7% while maintaining 97.7% accuracy. It can monitor cardiac arrhythmia on a long-term basis (3 years or 6 years). It can transfer the ECG data to a monitoring center. When the presence of AFib is detected, it can notify healthcare provider. It would inevitably take some time for the healthcare provider to contact the patient and to arrange for therapeutic intervention. One disadvantage is that it requires a surgery to insert the device under the skin of the chest. Another disadvantage is that it cannot automatically start therapeutic intervention immediately, when it is urgently needed. There exists a great need to have a non-invasive (not requiring surgery) ECG monitor that can monitor the heart continuously for a prolonged time. The auricular ECG monitoring system and smart-ring ECG monitoring system as disclosed in this invention will solve this issue and the problems with the smart watches.

Vagus nerve stimulation (VNS) therapy has been found by various studies to be effective in treating several types of cardiac arrhythmia, hypertension and heart failure. The placement of a traditional VNS device requires a surgery. The VNS device is usually placed under the skin in the upper chest with a wire going to the neck to wrap around or attach to the left vagus nerve in the neck. Various settings of stimulating strength, pattern, duration, frequency, timing and intervals have been studied to find the most effective stimulating parameters. The stimuli could also be adjusted for different time of the day or night.

Similar to VNS, transcutaneous auricular vagus nerve stimulation (taVNS or tVNS) has been studied for cardiac arrhythmia and was also found to be effective in treating cardiac arrhythmia. As compared with VNS, taVNS has the advantage that it does not require a surgery for placement. For most taVNS, its stimulating electrode is usually attached to the tragus or concha portion of the external ear and the stimulating electric current is administered transcutaneously in a way similar to transcutaneous electrical nerve stimulation (TENS). The skin of tragus, concha and the external ear canal is innervated by the auricular branch of vagus nerve. Liu C. et al reported in 2020 that taVNS stimulation is effective for cardiac arrhythmia, similar to VNS.

Various stimulation parameters for taVNS have been studied. The key parameters of taVNS include: duration, intensity of the electric stimuli, pulse width, pulse frequency and duty cycles and timing etc. In 2021 Kathrin Machetanz et al reported using taVNS for the heart. They applied taVNS to six different targets of the external ear, including cymba conchae, cavum conchae, outer tragus, inner tragus, crus helicis, and triangular fossa. They found that stimulation to cymba conchae, triangular fossa or inner tragus has strong effects. They applied 24 combinations of stimulus parameters, including 8 electrical charges per phase, 3 pulse durations and 8 charge-balanced current intensities, i.e., 100 microsecond (0.25-2 mA in steps of 0.25 mA), 260 microseconds (0.096-0.769 mA in steps of 0.096 mA) and 500 microseconds (0.050-0.400 mA in steps of 0.050 mA). Thirty bursts at each parameter combination were applied with a periodicity of 1 Hz. Each burst consisted of 5 pulses applied at 25 Hz. Left and right taVNS were studied. They found that 100 microsecond pulse duration and 2 mA current intensity are comfortable and effective.

In 2022, Giuseppe Forte et al reported using taVNS for the heart. They used a taVNS® L device which was developed by tVNS Technologies GmbH (Erlangen, Germany) which is approved with CE certification. It has a programmable stimulation unit connected to two titanium electrodes located in an earphone-like structure. Stimulation was applied to the left ear. The stimulation parameters were pre-set, with a biphasic impulse frequency at a rate of 25 Hz, width of 200-300 us and an on-off cycle of 30 seconds. They placed the stimulating electrodes at cymba conchae, because it had been shown to have the highest density of innervations from the auricular branch of the vagus nerve. There are various other studies using different stimulation parameters, as published by vagus.net.

The therapeutic benefits of taVNS were reviewed by George Bazoukis et al in 2023. It was found that stimulation to the vagus nerve, by either VNS or taVNS, is beneficial to various cardiovascular diseases, including arrhythmia, with improved clinical outcome.

The tragus and concha portions of the external ear have a unique physiological function because they have rich innervation of the autonomic nerve through the auricular branch of the vagus nerve. The auricular branch of the vagus nerve is an optimal location to place a vagus nerve stimulator. For traditional taVNS, a stimulating electrode is attached to the tragus, cymba concha or cavum concha. The stimulating electrode is connected with a long wire to a stimulating unit, similar to a TENS unit. The presence of a long wire makes it inconvenient. Some taVNS use hand-held or portable devices. However, a hand-held device has the disadvantage that it might not be readily available when it is needed urgently.

In 1997 the US Food and Drug Administration (FDA) approved cervical vagus nerve stimulation (with VNS) only for the left vagus nerve when stimulated at the neck region, due to concern of possible bradycardia when the right vagus nerve at neck region is stimulated. Traditionally, VNS devices at neck area (vagal stimulation at neck area) are typically implanted to stimulate the left vagus nerve due to concern of possibility for the right vagus nerve stimulation to have a potential adverse effect of bradycardia. However, many studies with taVNS have shown that bilateral auricular vagus nerve stimulation is safe. This is because the right cervical vagus nerve innervates the sinoatrial node, which regulates heart rhythm. The auricula branch of vagus nerve is a sensory nerve and does not have the right vagus nerve's cardiac motor efferent function. The practice of using transcutaneous auricular vagus nerve stimulation (taVNS) was reviewed by Yu Wang et al in 2020. They reviewed other studies and found that bilateral auricular vagus nerve stimulation is safe with no increase of side effects as compared with left-sided stimulation. They also stated that bilateral stimulation could be more effective. However, other studies comparing unilateral versus bilateral taVNS stimulation have shown mixed results. From review of many studies, they found that the stimulation parameters have a large range of variation. The most common stimulation frequency is 20 Hz or 25 Hz (range 0.5-120 Hz). The common stimulation pulse width is 1 ms or 0.25 ms (range 0.02-1 ms). They did find that pulse width of 500 microsecond is the most biological active. They found that the stimulation intensity is often adjusted by the patients according to their tolerance.

In 2024, Ashraf Gerges et al published a review article that also showed safety of bilateral auricular vagus nerve stimulation. They found that taVNS has favorable therapeutic effects on a wide range of clinical pathologies, including paroxysmal atrial fibrillation, heart failure, myocardial infarction, and several neuro-psychiatric diseases. They also described various electric stimulation parameters. The stimulation parameters include pulse frequency, pulse width, pulse-pause ratio, electrode type, device used, electric current type, electrode location etc. The Intensity ranged from 0.5 to 50 mA. The electrode size ranged from 2-200 mm. Pulse frequency of either 25 Hz or 20 Hz was used in 74% of the studies. The most common pulse width ranged from 0.05 to 1.0 ms, with either 0.20 or 0.25 ms. being the most commonly used. In their review, 62% of the studies stimulated only the left auricular branch of vagus nerve, while bilateral vagus nerve stimulation was done in 27% of the studies.

Recent advances in ECG analysis algorithms have helped detection of impending serious cardiac arrhythmia. Key predictive patterns of premature atrial contractions (PACs) have been found to help predict development of paroxysmal atrial fibrillation (AFib), including frequency of PACs (or PAC burden) (for example more than 3,000 PACs in a 24-hour period). Origin of PAC from pulmonary veins area is also a highly critical predictor. The pattern (clustering and timing) of PACs, including short rapid bursts or clusters of PACs, using clustering analysis algorithms and variable timing analysis algorithms can also help to predict paroxysmal atrial fibrillation. With artificial intelligence and machine learning, it has been shown that clustering PACs based on their P-wave morphology, timing, and coupling intervals can predict impending AFib. A high variability in the timing of PACs, especially in the coupling interval (the time between the premature beat and the preceding normal beat), has also been found to help predict impending AFib risk. Presence of these specific PACs patterns indicate atrial cardiomyopathy that make the patient vulnerable to atrial fibrillation.

Similarly, key predictive patterns of premature atrial contractions (PACs) have been found to help predict development of atrial flutter (AFL). These include frequency of PACs (PAC burden) (for example over 3,000 PACs in 24 hours). The temporal patterns of PACs are also correlated with risk of impending atrial flutter. With artificial intelligence and machine learning to analyze ECG data, distinct clusters (high-risk clusters) of PACs have been identified, including the PACs morphology, timing, and coupling interval and these have been developed into ECG analysis algorithms for prediction of impending AFL. Source of the PACs (from pulmonary vein area) also helps in prediction of atrial flutter.

Likewise, key predictive patterns of premature atrial contractions (PACs) have been found to help predict development of supraventricular tachycardia (SVT). Frequent PACs (for example: over 3,000 PACs in 24 hours) is again indicative of high risk for SVT. The PAC patterns can also help to predict SVT, including repetitive and organized PAC patterns significantly increase the risk of SVT as compared to simple, isolated PACs, including bigeminy and trigeminy. Occasional short run of non-sustained SVT can also predict impending sustained SVT. Early PACs that are blocked (not conducted to the ventricles) can also be a significant risk factor for impending more complex SVT.

There are ECG patterns that can help to predict impending ventricular tachycardia (VT). VT may occur with underlying cardiac diseases, for example Brugada syndrome with its specific ECG patterns (prominent, coved ST-segment elevation of at least 2 mm in leads V1 and V2 with an associated T-wave inversion.) or arrhythmogenic right ventricular cardiomyopathy (ECG showing T-wave inversion, Epsilon wave, prolong S-wave upstroke). Other ECG technics, like signal-averaged ECG and heart-rate variability may also help to predict impending ventricular tachycardia (VT). Frequent premature ventricular contractions (PVCs) or high burden of PVCs, especially with uniform morphology, can help to predict impending VT. Algorithms for differentiating VT from other wide-complex tachycardias are known in the art.

Similarly, there are ECG patterns and features that can help predict impending ventricular fibrillation (VF). These ECG markers may include T-wave alternans, prolonged T-peak to T-end interval, QT interval prolongation, early repolarization pattern, fragmented QRS, long QRS duration, Epsilon waves, frequent PVCs or episodes of non-sustained ventricular tachycardia, etc. and presence of these may indicate increased risks of impending ventricular fibrillation.

Therefore, a need exists for a novel wearable convenient and non-invasive ECG monitoring system that can monitor the heart of a wearer for a long time. A need also exists for a novel automatic anti-arrhythmia system which can automatically initiate therapy for serious cardiac arrhythmia or impending serious cardiac arrhythmia by integrating a wearable ECG monitoring system and a transcutaneous auricular vagus nerve stimulation unit.

### BRIEF SUMMARY OF THE INVENTION

According to one aspect consistent with the principles of the invention, an auricular electrocardiogram (ECG) monitoring system that is wearable, convenient, suitable for long-term monitoring and with much less muscle artifacts and movement artifacts is disclosed. In some embodiments, an auricular ECG monitoring system may be configured as a single-ear auricular ECG system. The single-ear auricular ECG system may comprise an ECG recording module. The ECG recording module may comprise at least two wired or wireless ECG electrodes (ECG sensor electrodes) in electronic communication with the ECG recording module. All of the ECG electrodes (for example a first auricular ECG electrode and a second auricular ECG electrode) may be configured to be coupled to a wearer's first ear or a peri-auricular area around the wearer's first ear. When the first and second auricular ECG electrodes are coupled to the wearer's first ear or the peri-auricular area around the wearer's first ear the first and the second auricular ECG electrodes are positioned in contact with separate skin locations on at least one area of the wearer's skin, the area selected from at least one of the following: external ear of the wearer's first ear, external ear canal of the wearer's first ear, and the peri-auricular area around the wearer's first ear. (It should be noted that the first and the second auricular ECG electrodes may contact separate skin locations within a single area. Alternatively, the first auricular ECG electrode may contact a skin location within one area and the second auricular ECG electrode may contact a separate skin location within another area.) These skin locations that the auricular ECG electrodes are attached to may be adequately separated (with different angles or directions) relative to the location of the heart of the wearer so that they can pick up some differences of the cardiac action potentials in order to enable the ECG recording module to perform an ECG. The ECG recording module may be configured to record ECG data of the wearer via the auricular ECG electrodes and the ECG recording module may be in electronic communication with a processing unit. With the help of ECG analysis algorithms, the processing unit may be configured to record and analyze the ECG data from the ECG recording module to assess the wearer's ECG profile. The processing unit may be configured to analyze ECG data recorded by the ECG recording module to detect presence or cessation of a serious cardiac arrhythmia of the wearer. The processing unit may be further configured to analyze the ECG data recorded by the ECG recording module to detect the presence or cessation of an impending serious cardiac arrhythmia of the wearer. The serious cardiac arrhythmia may include atrial fibrillation (AFib), atrial flutter (AFL), supraventricular tachycardia (SVT), ventricular tachycardia (VT), and ventricular fibrillation (VF).

As used herein, a peri-auricular area refers to a portion of the head around the auricle (pinna) and this portion of the head is typically hairless. The peri-auricular area includes a portion of the head in front of the auricle (pre-auricular area) and a portion of the head above and behind the auricle (post-auricular area). The pre-auricular area is small, about one inch wide and two inches long and curved along the anterior edge of the auricle. The post-auricular area is also small, about one inch wide and about three inches long and curved along the superior and posterior edges of the auricle (pinna). The post-auricular area is where a behind-the-ear hearing aid is usually located. The small pre-auricular area and post-auricular area together will be called “peri-auricle area” hereinafter. (Anterior, posterior, superior, in front of and behind etc. all refer to the directions relative to the wearer's head when the wearer is in an upright position.) As referred to herein, the external ear canal refers to the part of the ear that connects the visible outer ear (pinna) to the middle ear, essentially the tube that carries sound waves to the eardrum.

In some embodiments, an auricular ECG monitoring system may be configured as a dual-ear auricular ECG system. The dual-ear auricular ECG system may comprise an ECG recording module. The ECG recording module may comprise at least two wired or wireless ECG electrodes in electronic communication with the ECG recording module. At least one of the ECG electrodes (for example a first auricular ECG electrode) may be configured to be coupled to a wearer's first ear or a peri-auricular area around the wearer's first ear. When the first auricular ECG electrode is coupled to the wearer's first ear or the peri-auricular area around the wearer's first ear the first auricular ECG electrode is positioned in contact with an area of the wearer's skin, the area selected from at least one of the following: external ear of the wearer's first ear, external ear canal of the wearer's first ear, and the peri-auricular area around the wearer's first ear. At least another one of the ECG electrodes (for example a contralateral auricular ECG electrode) may be configured to be coupled to the wearer's second ear or a peri-auricular area around the wearer's second ear. When the contralateral ECG electrode is coupled to the wearer's second ear or the peri-auricular area around the wearer's second ear the contralateral auricular ECG electrode is positioned in contact with an area of the wearer's skin, the area selected from at least one of the following: external ear of the wearer's second ear, external ear canal of the wearer's second ear, and the peri-auricular area around the wearer's second ear. In some embodiments, all of the ECG electrodes, including the first auricular ECG electrode and the contralateral auricular ECG electrode, are configured as wired or wireless ECG electrodes and are in wired or wireless communication with the ECG recording module. The ECG recording module may comprise a wired or wireless amplifier. The ECG recording module may be configured to record ECG data of the wearer via all of the wired or wireless ECG electrodes (including the wired or wireless first auricular ECG electrode and the wired or wireless contralateral auricular ECG electrode) and the ECG recording module may be in electronic communication with a processing unit. With the help of ECG analysis algorithms, the processing unit may be configured to record and analyze the ECG data from the ECG recording module to assess the wearer's ECG profile. The processing unit may be further configured to analyze ECG data recorded by the ECG recording module to detect the presence or cessation of a serious cardiac arrhythmia and the presence or cessation of an impending serious cardiac arrhythmia of the wearer. The serious cardiac arrhythmia may include atrial fibrillation (AFib or AF), supraventricular tachycardia (SVT), atrial flutter (AFL), ventricular tachycardia (VT), and ventricular fibrillation (VF).

According to another aspect consistent with the principles of the invention, an auricular automatic anti-arrhythmia system is disclosed. Preferably, the auricular automatic anti-arrhythmia system may comprise an auricular electrocardiogram (ECG) monitoring system, a transcutaneous auricular vagus nerve stimulation unit (taVNS unit) and a processing unit. The auricular ECG monitoring system may be configured as a single-ear auricular ECG system or a dual-ear auricular ECG system, as described hereinbefore. The auricular electrocardiogram (ECG) monitoring system may include an ECG recording module that is configured to record ECG data of the wearer. The taVNS unit may have a stimulating electrode attached to vagus innervated auricular skin of the wearer. (Vagus innervated auricular skin refers to areas of auricular skin that have rich innervation from the auricular branch of vagus nerve, such as external ear canal, tragus, cymba concha, cavum concha and small adjacent areas of the ear). When prompted by signals from a processing unit, the taVNS unit is configured to generate electric stimulation to the vagus innervated auricular skin via its stimulating electrode. The auricular electrocardiogram (ECG) monitoring system may be in electronic communication with the processing unit. The processing unit may be configured to analyze ECG data recorded by the ECG recording module to detect presence or cessation of a serious cardiac arrhythmia. When presence of a serious cardiac arrhythmia is detected, the processing unit may be further configured to immediately send signals to the taVNS unit and the taVNS unit is configured to automatically start sending pre-determined electric stimuli to an auricular branch of a vagus nerve of the wearer to alleviate or stop the serious cardiac arrhythmia. When cessation of ECG signals suggestive of a serious cardiac arrhythmia is detected by the processing unit, the processing unit is configured to send signals to the taVNS unit to stop sending electric stimulation to the auricular branch of vagus nerve. The processing unit may be further configured to analyze ECG data recorded by the ECG recording module to detect presence or cessation of an impending serious cardiac arrhythmia. When presence of an impending serious cardiac arrhythmia is detected, the processing unit may be further configured to immediately send signals to the ta VNS unit and the taVNS unit is configured to automatically start sending pre-determined electric stimuli to an auricular branch of a vagus nerve of the wearer to alleviate or stop the impending serious cardiac arrhythmia. When cessation of ECG signals suggestive of an impending serious cardiac arrhythmia is detected by the processing unit, the processing unit is configured to send signals to the taVNS unit to stop sending electric stimulation to the auricular branch of vagus nerve. It should be noted that the auricular automatic anti-arrhythmia system is wearable and contains self-installable ECG electrodes and self-installable stimulating electrode of the taVNS unit, due to the features of the housing material and housing set-ups, details to be described hereinafter.

According to yet another aspect consistent with the principles of the invention, a chest-based automatic anti-arrhythmia system is disclosed. Preferably, the chest-based automatic anti-arrhythmia system may include a chest-based electrocardiogram (ECG) monitoring system having an ECG recording module and a plurality of chest-contacting ECG electrodes in contact with the chest of a wearer, and the plurality of ECG electrodes may be in electronic communication with the ECG recording module. A transcutaneous auricular vagus nerve stimulation unit (taVNS unit) may have a stimulating electrode attached to vagus innervated auricular skin of the wearer. The ECG recording module of the chest-based electrocardiogram (ECG) monitoring system and the taVNS unit may be in electronic communication with the processing unit. The processing unit may be configured to analyze ECG data recorded by the ECG recording module to detect presence or cessation of a serious cardiac arrhythmia; and when presence of a serious cardiac arrhythmia is detected by the processing unit, the processing unit may be further configured to immediately send signals to the taVNS unit and the taVNS unit is configured to automatically start sending pre-determined electric stimuli to an auricular branch of a vagus nerve of the wearer to alleviate or stop the serious cardia arrhythmia. The processing unit may be further configured to analyze ECG data recorded by the ECG recording module to detect presence or cessation of an impending serious cardiac arrhythmia; and when presence of an impending serious cardiac arrhythmia is detected by the processing unit, the processing unit may be further configured to immediately send signals to the taVNS unit and the taVNS unit is configured to automatically start sending pre-determined electric stimuli to an auricular branch of a vagus nerve of the wearer to alleviate or stop the impending serious cardia arrhythmia.

In alternative embodiments, an arm-based automatic anti-arrhythmia system may include an arm-based electrocardiogram (ECG) monitoring system having an ECG recording module and a plurality of ECG electrodes (arm-contacting ECG electrodes) in contact with skin of an arm (preferably the left upper arm) of a wearer, and the plurality of ECG sensor electrodes may be in electronic communication with the ECG recording module. A transcutaneous auricular vagus nerve stimulation unit (taVNS unit) may have a stimulating electrode attached to vagus innervated auricular skin of the wearer. The ECG recording module of the arm-based electrocardiogram (ECG) monitoring system and the taVNS unit may be in electronic communication with the processing unit. The processing unit may be configured to analyze ECG data recorded by the ECG module to detect presence or cessation of a serious cardiac arrhythmia; and when presence of a serious cardiac arrhythmia is detected by the processing unit, the processing unit may be further configured to immediately send signals to the taVNS unit and the taVNS unit is configured to automatically start sending pre-determined electric stimuli to an auricular branch of a vagus nerve of the wearer. The processing unit may be further configured to analyze ECG data recorded by the ECG recording module to detect presence or cessation of an impending serious cardiac arrhythmia. When presence of an impending serious cardiac arrhythmia is detected by the processing unit, the processing unit may be further configured to immediately send signals to the taVNS unit and the taVNS unit is configured to automatically start sending pre-determined electric stimuli to an auricular branch of a vagus nerve of the wearer.

According to a further aspect consistent with the principles of the invention, smart-ring electrocardiogram (ECG) monitoring systems and smart-ring automatic anti-arrhythmia systems are disclosed. In some embodiments, a smart-ring ECG monitoring system may be configured as an ear-finger ECG system or a finger-finger ECG system. In other embodiments, a smart-ring automatic anti-arrhythmia system may be configured as an ear-finger automatic anti-arrhythmia system or a finger-finger automatic anti-arrhythmia system. The novel ear-finger automatic anti-arrhythmia system may be configured to comprise an ear-finger ECG system, a transcutaneous auricular vagus verve stimulation (taVNS) unit and a processing unit. Similarly, the novel finger-finger automatic anti-arrhythmia system may be configured to comprise a finger-finger ECG system, a taVNS unit and a processing unit. In alternative embodiments, a smart-ring ECG monitoring system may be configured as an ear-toe ECG system or a finger-toe ECG system. In other embodiments, a smart-ring automatic anti-arrhythmia system may be configured as an ear-toe automatic anti-arrhythmia system or a finger-toe automatic anti-arrhythmia system. The novel ear-finger automatic anti-arrhythmia system may be configured to comprise an ear-finger ECG system, a transcutaneous auricular vagus verve stimulation (taVNS) unit and a processing unit. Similarly, the novel finger-finger automatic anti-arrhythmia system may be configured to comprise a finger-finger ECG system, a taVNS unit and a processing unit. Likewise, the novel ear-toe automatic anti-arrhythmia system may be configured to comprise an ear-toe ECG system, a transcutaneous auricular vagus verve stimulation (taVNS) unit and a processing unit. Similarly, the novel finger-toe automatic anti-arrhythmia system may be configured to comprise a finger-toe ECG system, a taVNS unit and a processing unit.

In some embodiments, an ear-finger ECG system may comprise at least two wired or wireless ECG electrodes. At least one of the ECG electrodes (for example a first finger-ring electrode) is configured to be placed on an inner surface of a first finger-ring to be worn on a finger (for example an X-finger) of a wearer's first hand. The first finger-ring electrode is configured to be located on an inner surface of the first finger-ring and the first finger-ring is configured to be made of an elastic adaptable and flexible material (such as silicone-type material) and the elastic adaptable flexible material is configured to have appropriate size, elasticity, adaptability and flexibility such that the first finger-ring will snugly contact the skin of the X-finger of the wearer's first hand when the wearer wears the first finger-ring on the X-finger of the wearer's first hand. The first finger-ring electrode is configured to be partially embedded in the inner surface of the first finger-ring with slight protrusion at the inner surface of the first finger-ring such that the first finger-ring electrode will naturally snugly contact the skin of the X-finger of the wearer's first hand when the first finger-ring is worn on the X-finger of the wearer's first hand. Thus, the first finger-ring electrode can be easily self-installed and self-removed by the wearer (user) and there is no need to have a technologist to perform installation or removal of the ECG electrode. The ear-finger ECG system further comprises at least another ECG electrode (for example a first auricular electrode) to be placed on a surface of an auricular housing structure that is configured to be worn on or in an ear of the wearer. The auricular housing structure includes an in-the-ear portion that is configured to be in contact with the wearer's external ear canal and the bowl-shaped area of tragus-concha when the in-the-ear portion is put in place into a wearer's external ear canal and the bowl-shaped area of tragus-concha. The auricular housing structure may be selected from one of the following: an earbud-style structure, a behind-the-ear-hearing-aid-style structure, or a tubular-shaped structure. Optionally, a taVNS housing may be configured to be integrated into or with the auricular housing structure. The in-the-ear portion of the auricular housing structure is configured to comprise an elastic, adaptable and flexible material (such as soft foam or soft silicone-type material.). The first auricular electrode is configured to be partially embedded in the surface of the in-the-ear portion with slight protrusion at the surface of the in-the-ear portion of the auricular housing structure. The in-the-ear portion of the auricular housing structure is configured to have appropriate size, elasticity, adaptability and flexibility such that the in-the-ear portion will naturally snugly fill the interior of the wearer's external ear canal and the bowl-shaped area of tragus-concha when the in-the-ear portion is put in place into the wearer's external ear canal and the bowl-shaped area of tragus-concha and such that the first auricular electrode will naturally snugly contact the skin of the wearer's external ear canal or skin of the bowl-shaped area of tragus-concha when the in-the-ear portion is put in place. Installing and removing the first auricular electrode will be as easy as inserting and removing the in-the-ear portion from the wearer's external ear. This feature of easy self-installation and easy self-removal will be very convenient for the wearer. The ear-finger ECG system may further comprise an ECG recording module that may be housed either within the first finger-ring or within the auricular housing structure. The first finger-ring electrode and the first auricular electrode are configured to be wired or wireless ECG electrodes. The ECG recording module is configured to comprise a wired or wireless ECG amplifier. The first finger-ring electrode and the first auricular electrode are configured to be in wired or wireless (preferably wireless) communication with the ECG recording module and the ECG recording module is configured to record ECG data of the wearer via the first finger-ring electrode and the first auricular electrode. The processing unit is configured to analyze the ECG data recorded by the ECG recording module to detect presence or cessation of any serious cardiac arrhythmia (including AFib, AFL, SVT, VT and VF). The processing unit is further configured to analyze the ECG data recorded by the ECG recording module to detect presence or cessation of any impending serious cardiac arrhythmia. The ear-finger ECG system may further comprise a network interface and one of the following: a speaker, a vibrator and a display screen on a client device of the wearer. The network interface is in electronic communication with the processing unit, the speaker, the vibrator and the display screen on a client device of the wearer. When the processing unit detects presence of a serious cardiac arrhythmia, the processing unit is configured to send signals to at least one of the following: the speaker (that is configured to generate an audible warning notification), the vibrator (that is configured to generate a tactile warning notification) and the display screen (that is configured to generate a visible warning notification) on the client device of the wearer to alert the wearer to take appropriate action. Additionally, the processing unit may be configured to send visible notification to a display screen of a client device of the wearer's healthcare provider to alert the healthcare provider to take appropriate action. When the processing unit detects presence of an impending serious cardiac arrhythmia, the processing unit is configured to send signals to at least one of the following: the speaker (that is configured to generate an audible warning notification), the vibrator (that is configured to generate a tactile warning notification) and the display screen (that is configured to generate a visible warning notification) on the client device of the wearer to alert the wearer to take appropriate action. Additionally, the processing unit may be configured to send visible notification to a display screen of a client device of the wearer's healthcare provider to alert the healthcare provider to take appropriate action.

The aforementioned features and setups of the first finger-ring serving the housing purpose for the first finger-ring electrode enable the first finger-ring electrode to become easily self-installable and self-removable by the wearer (user) and there is no need for another person or a technologist to install or to remove the ECG electrode. Applying adhesive material to secure the ECG electrode is also not needed. Similarly, these features and setups of the in-the-ear portion of the auricular housing structure to serve the housing purpose for the first auricular electrode enable the first auricular electrode to become easily self-installable and self-removable by the wearer (user) and there is no need for another person or a technologist to perform installation or removal of the ECG electrodes. Applying adhesive material to secure the ECG electrode is also unneeded. As used herein, a housing structure for ECG electrodes that can be easily self-installed and self-removed by the wearer (user) will be referred to as “self-installable-housing structure”. Examples of self-installable-housing structures may include the in-the-ear portion of the auricular housing structure, an earbud-style structures or hearing-aid-style structure when in use (when inserted inside an external ear canal) and ring-type structures for fingers or toes.

In some embodiments, a finger-finger ECG system may comprise at least two wired or wireless ECG electrodes. At least one of the ECG electrodes (for example a first finger-ring electrode) is configured to be placed on an inner surface of a first finger-ring to be worn on a finger (for example an X-finger) of the wearer's first hand. The finger-finger ECG system further comprises at least another ECG electrode (for example a second finger-ring electrode) to be placed on an inner surface of a second finger-ring that is configured to be worn on a finger (for example a Y-finger) of the wearer's second hand. The first finger-ring may be configured to be made of elastic flexible adaptable material (for example silicone-type material) and the elastic flexible adaptable material is configured to have appropriate size, elasticity, adaptability and flexibility such that the first finger-ring will snugly fit the X-finger of the wearer's first hand when the wearer wears the first finger-ring on the X-finger of the wearer's first hand. The first finger-ring electrode is configured to be partially embedded in the inner surface of the first finger-ring with slight protrusion at the inner surface of the first finger-ring such that the first finger-ring electrode will snugly contact the skin of the X-finger of the wearer's first hand when the first finger-ring is worn by the wearer. Likewise, the second finger-ring electrode is configured to be placed on the inner surface of the second finger-ring and the second finger-ring is configured to be made of elastic flexible adaptable material and the elastic flexible adaptable material is configured to have appropriate size, flexibility, adaptability and elasticity such that the second finger-ring electrode will snugly fit the Y-finger of the wearer's second hand when the wearer wears the second finger-ring on the Y-finger of the wearer's second hand. The second finger-ring electrode is configured to be partially embedded in the inner surface of the second finger-ring with slight protrusion at the inner surface of the second finger-ring such that the second finger-ring electrode will naturally contact the skin of the Y-finger of the wearer's second hand when the second finger-ring is worn by the wearer. The first finger-ring electrode and the second finger-ring electrode are configured as wired or wireless ECG electrodes. The ECG recording module is configured to comprise wired or wireless ECG amplifier. The first finger-ring electrode and the second finger-ring electrode are configured to be in wired or wireless communication (preferably wireless) with the ECG recording module and the ECG recording module is configured to record ECG data of the wearer via the first finger-ring electrode and the second finger-ring electrode. The processing unit is configured to analyze the ECG data recorded by the ECG recording module to assess the wearer's ECG profile and to detect presence or cessation of any serious cardiac arrhythmia (including AFib, AFL, SVT, VT, and VF) or any impending serious cardiac arrhythmia. The finger-finger ECG system may further comprise a network interface and one of the following: a speaker, a vibrator and a display screen on a client device of the wearer. The network interface is in electronic communication with the processing unit, the speaker, the vibrator and the display screen on a client device of the wearer. When the processing unit detects presence of a serious cardiac arrhythmia, the processing unit is configured to send signals to at least one of the following: the speaker (that is configured to generate an audible warning notification), the vibrator (that is configured to generate a tactile warning notification) and the display screen (that is configured to generate a visible warning notification) on the client device of the wearer to alert the wearer to take appropriate action. Additionally, the processing unit may be configured to send visible notification to a display screen of a client device of the wearer's healthcare provider to alert the healthcare provider to take appropriate action. When the processing unit detects presence of an impending serious cardiac arrhythmia, the processing unit is configured to send signals to at least one of the following: the speaker (that is configured to generate an audible warning notification), the vibrator (that is configured to generate a tactile warning notification) and the display screen (that is configured to generate a visible warning notification) on the client device of the wearer to alert the wearer to take appropriate action. Additionally, the processing unit may be configured to send visible notification to a display screen of a client device of the wearer's healthcare provider to alert the healthcare provider to take appropriate action.

In some embodiments, an ear-finger automatic anti-arrhythmia system may be configured to comprise an ear-finger ECG system, a transcutaneous auricular vagus verve stimulation (taVNS) unit and a processing unit. The ear-finger ECG system and the taVNS unit are as described hereinbefore. The taVNS unit comprises a stimulating electrode that is configured to contact vagus-innervated auricular skin (including one of: tragus, cavum concha, cymba concha and external ear canal). The processing unit is in electronic communication (preferably wireless communication) with the taVNS unit and the ear-finger ECG system. The processing unit is configured to process the ECG data transmitted from the ear-finger ECG system and the processing unit is configured to assess the wearer's ECG profile and to detect presence or cessation of serious cardiac arrhythmia (including AFib, AFL, SVT, VT, and VF). When serious cardiac arrhythmia is detected by the processing unit, the processing unit is further configured to send signals (arrhythmia-warning signals) to the taVNS unit to prompt the taVNS unit to start sending pre-determined electric stimulation to the vagus innervated auricular skin to which the stimulating electrode of the taVNS unit is in contact with, similar to descriptions hereinbefore. (Examples for pre-determined electric stimulation parameters for the taVNS unit for serious cardiac arrhythmia are shown on Table 1.). When impending serious cardiac arrhythmia is detected by the processing unit, the processing unit is further configured to send signals (impending-arrhythmia-warning signals) to the taVNS unit to prompt the taVNS unit to start sending pre-determined electric stimulation to the vagus innervated auricular skin to which the stimulating electrode of the taVNS unit is in contact with, similar to descriptions hereinbefore. (Examples for pre-determined electric stimulation parameters for the taVNS unit for impending serious cardiac arrhythmia are shown on Table 2.) When cessation of the serious cardiac arrhythmia or the impending serious cardiac arrhythmia is detected by the processing unit, the processing unit is further configured to send arrhythmia-cessation signals or impending-arrhythmia-cessation signals to the taVNS unit to cause the taVNS unit to stop sending pre-determined electric stimuli. The ear-finger automatic anti-arrhythmia system may further comprise a network interface and one of the following: a speaker, a vibrator and a display screen on a client device of the wearer. The network interface is in electronic communication with the processing unit, the speaker, the vibrator, and the display screen on a client device of the wearer. When the processing unit detects presence of a serious cardiac arrhythmia, the processing unit is configured to send signals to the speaker (that is configured to generate an audible warning notification), the vibrator (that is configured to generate a tactile warning notification) and the display screen (that is configured to generate a visible warning notification) on the client device of the wearer to alert the wearer to take appropriate action. Additionally, the processing unit may be configured to send visible notification to a display screen of a client device of the wearer's healthcare provider to alert the healthcare provider to take appropriate action. When the processing unit detects presence of an impending serious cardiac arrhythmia, the processing unit is configured to send signals to the speaker (that is configured to generate an audible warning notification), the vibrator (that is configured to generate a tactile warning notification) and the display screen (that is configured to generate a visible warning notification) on the client device of the wearer to alert the wearer to take appropriate action. Additionally, the processing unit may be configured to send visible notification to a display screen of a client device of the wearer's healthcare provider to alert the healthcare provider to take appropriate action.

In some embodiments, a finger-finger automatic anti-arrhythmia system may be configured to comprise a finger-finger ECG system, a transcutaneous auricular vagus verve stimulation (taVNS) unit and a processing unit. The finger-finger ECG system and the ta VNS unit are as described hereinbefore. The taVNS unit comprises a stimulating electrode that is configured to contact vagus-innervated auricular skin (including one of: tragus, cavum concha, cymba concha and external ear canal). The processing unit is in electronic communication (preferably wireless communication) with the taVNS unit and the finger-finger ECG system. The processing unit is configured to process the ECG data transmitted from the finger-finger ECG system and the processing unit is configured to assess the wearer's ECG profile and to detect presence or cessation of a serious cardiac arrhythmia or an impending serious cardiac arrhythmia (including AFib, AFL, SVT, VT and VF). When a serious cardiac arrhythmia is detected by the processing unit, the processing unit is further configured to send signals (arrhythmia-warning signals) to the taVNS unit to cause the taVNS unit to start sending pre-determined electric stimulation to the vagus innervated auricular skin to which the stimulating electrode of the taVNS unit is in contact with (stimulating parameters as shown in Table 1). When an impending serious cardiac arrhythmia is detected by the processing unit, the processing unit is further configured to send signals (impending-arrhythmia-warning signals) to the taVNS unit to cause the taVNS unit to start sending pre-determined electric stimulation to the vagus innervated auricular skin to which the stimulating electrode of the taVNS unit is in contact with (stimulating parameters as shown in Table 2). When cessation of the serious cardiac arrhythmia or cessation of the impending serious cardiac arrhythmia is detected by the processing unit, the processing unit is further configured to send signals (arrhythmia-cessation signals or impending-arrhythmia-cessation signals) to the taVNS unit to cause the taVNS unit to stop or modify sending pre-determined electric stimuli. The finger-finger automatic anti-arrhythmia system may further comprise a network interface and one of the following: a speaker, a vibrator and a display screen on a client device of the wearer. The network interface is in electronic communication with the processing unit, the speaker, the vibrator and the display screen on a client device of the wearer. When the processing unit detects presence of a serious cardiac arrhythmia or an impending serious cardiac arrhythmia, the processing unit is configured to send signals to the speaker (that is configured to generate an audible warning notification), the vibrator (that is configured to generate a tactile warning notification) and the display screen (that is configured to generate a visible warning notification) on the client device of the wearer to alert the wearer to take appropriate action. The processing unit may be further configured to send visible notification to a display screen of a client device of the wearer's healthcare provider to alert the healthcare provider to take appropriate action.

In some embodiments, a smart ring with an ECG electrode may be worn on a toe to have a novel ear-toe ECG monitoring system or a novel finger-toe ECG monitoring system, similar to descriptions hereinbefore. In some embodiments, a novel ear-toe automatic anti-arrhythmia system may be configured to comprise an ear-toe ECG system, a transcutaneous auricular vagus verve stimulation (taVNS) unit and a processing unit. Also, a novel finger-toe automatic anti-arrhythmia system may be configured to comprise a finger-toe ECG system, a taVNS unit and a processing unit, similar to descriptions hereinbefore. (Alternatively, an ECG electrode may be mounted on an ankle band to be worn on an ankle or a wrist band to be worn on a wrist to have similar functions as an ECG monitoring system and an automatic anti-arrhythmia system.)

As used herein, the term “auricular housing structure” refers to housing structures in or around the ear. (Examples of an auricular housing structure include an earbud-style structure, a behind-the-ear-hearing-aid-style structure, a tubular-shaped structure and a housing structure used for a taVNS unit.) An auricular housing structure may comprise as one of: an earbud style structure, a tubular-shaped structure, or a behind-the-ear-hearing-aid-style structure. Also, as used herein, the term “in-the-ear portion” of an auricular housing structure refers to a portion of the auricular housing structure that is put inside (or into) an external ear canal and a bowl-shaped area of tragus-concha when in use. As used herein, the term “bowl-shaped area of tragus-concha” refers to the area surrounding the opening of the external ear canal and this area is bowl-shaped and is formed by inner tragus and cavum concha. (cymba concha is above the cavum concha and they are separated by crus of the helix). The “in-the-ear portion” of an auricular housing structure is essentially the same as an in-the-ear portion of a behind-the-ear-hearing-aid-style structure (a behind-the-ear-hearing-aid typically includes an in-the-ear portion and a behind-the-ear portion.) Additionally, as used herein, the term “horizontal portion” of an earbud-style structure refers to the “nozzle” and “ear-tip” that are inserted inside an external ear canal and the “body” of the earbud that sit at the opening of the external ear canal (the “body” of an earbud is essentially sitting on the “bowl-shaped area of tragus-concha”) when an earbud-style structure is put in place. Thus, as used herein, the “horizontal portion” of an earbud-style structure is essentially the same as the “in-the-ear portion” of an auricular housing structure. The tubular-shaped structure itself is a housing structure located inside an external ear canal and thus is part of the “in-the-ear portion” of the auricular housing structure. It should be noted that, as described in detail hereinafter, the material and setups of the housing structures in this invention will enable easy self-installation and easy self-removal of the auricular electrodes and ring electrodes by the wearer (user) and there is no need for a certified technologist to perform that.

As used herein, the terms “self-installable” or “self-removable” electrode refer to an electrode (ECG electrodes or stimulating electrode of a taVNS unit) that can be installed or removed by an ordinary adult wearer (or user) himself or herself without any help from other person or from a technologist (no need for the technologist to prepare the skin surface and no need to use special adhesive material to attach the electrodes to the skin). The terms “self-installation” or “self-removal” of an electrode refer to installing or removing the electrode by an ordinary adult wearer (or user) himself/herself without any help from a technologist or other person. Likewise, the term “self-installable and self-removable” system refer to a system that uses self-installable and/or self-removable electrodes.

### BRIEF DESCRIPTION OF THE DRAWINGS

Some embodiments of the present invention are illustrated as an example and are not limited by the figures of the accompanying drawings, in which like references may indicate similar elements and in which:

FIG. 1-FIG. 1 depicts a diagram of an example of an auricular electrocardiogram (ECG) monitoring system according to various embodiments described herein.

FIG. 2-FIG. 2 illustrates a diagram of an example of an auricular automatic anti-arrhythmia system according to various embodiments described herein.

FIG. 3-FIG. 3 depicts an elevation view of the anatomy of a human external ear, including the locations of tragus, cymba-concha, cavum-concha, triangular fossa, helix, lobule, external ear canal, and peri-auricular area, etc.

FIG. 4-FIG. 4 illustrates a diagram of another example of an auricular electrocardiogram (ECG) monitoring system, with an earbud-style housing, according to various embodiments described herein.

FIG. 5-FIG. 5 shows a diagram of still another example of an auricular electrocardiogram (ECG) monitoring system, with a behind-the-ear-hearing-aid-style housing, according to various embodiments described herein.

FIG. 6-FIG. 6 depicts a diagram of a further example of an auricular electrocardiogram (ECG) monitoring system, with a tubular-shaped housing, according to various embodiments described herein.

FIG. 7-FIG. 7 illustrates a diagram of an example of an auricular automatic anti-arrhythmia system, with an earbud-style housing, according to various embodiments described herein.

FIG. 8-FIG. 8 shows a diagram of another example of an auricular automatic anti-arrhythmia system, with a behind-the-ear-hearing-aid-style housing, according to various embodiments described herein.

FIG. 9-FIG. 9 depicts a diagram of a further example of an auricular automatic anti-arrhythmia system, with a tubular-shaped housing, according to various embodiments described herein.

FIG. 10-FIG. 10 illustrates a block diagram illustrating some exemplary components of a single-ear auricular ECG system (as an example of an auricular ECG monitoring system) according to various embodiments described herein.

FIG. 11-FIG. 11 shows a block diagram illustrating some exemplary components of a dual-ear auricular ECG system (as another example of an auricular ECG monitoring system) according to various embodiments described herein.

FIG. 12-FIG. 12 illustrates a block diagram showing some exemplary components of an example of a processing unit for an ECG monitoring system according to various embodiments described herein.

FIG. 13-FIG. 13 depicts a block diagram illustrating some exemplary components of an example of a transcutaneous auricular vagus nerve stimulation (taVNS) unit according to various embodiments described herein.

FIG. 14-FIG. 14 illustrates a block diagram showing some exemplary components of an example of a chest-based ECG monitoring system according to various embodiments described herein.

FIG. 15-FIG. 15 shows a block diagram illustrating some exemplary components of an example of a client device according to various embodiments described herein.

FIG. 16-FIG. 16 depicts a diagram illustrating some exemplary components of an example of an auricular electrocardiogram (ECG) monitoring system, an auricular automatic anti-arrhythmia system, a chest-based automatic anti-arrhythmia system, an arm-based automatic anti-arrhythmia system, an ear-finger automatic anti-arrhythmia system, a finger-finger automatic anti-arrhythmia system, an ear-toe automatic anti-arrhythmia system and a finger-toe automatic anti-arrhythmia system, with their network connections, according to various embodiments described herein.

FIG. 17-FIG. 17 shows a diagram of an example of an arm-based automatic anti-arrhythmia system according to various embodiments described herein.

FIG. 18-FIG. 18 illustrates a block diagram showing some exemplary components of an example of an arm-based ECG monitoring system according to various embodiments described herein.

FIG. 19-FIG. 19 illustrates a diagram of an example of a chest-based automatic anti-arrhythmia system according to various embodiments described herein.

FIG. 20-FIG. 20 shows a schematic diagram of some exemplary components of an example of an auricular automatic anti-arrhythmia system, having a single-ear auricular ECG system and a taVNS unit, according to various embodiments described herein.

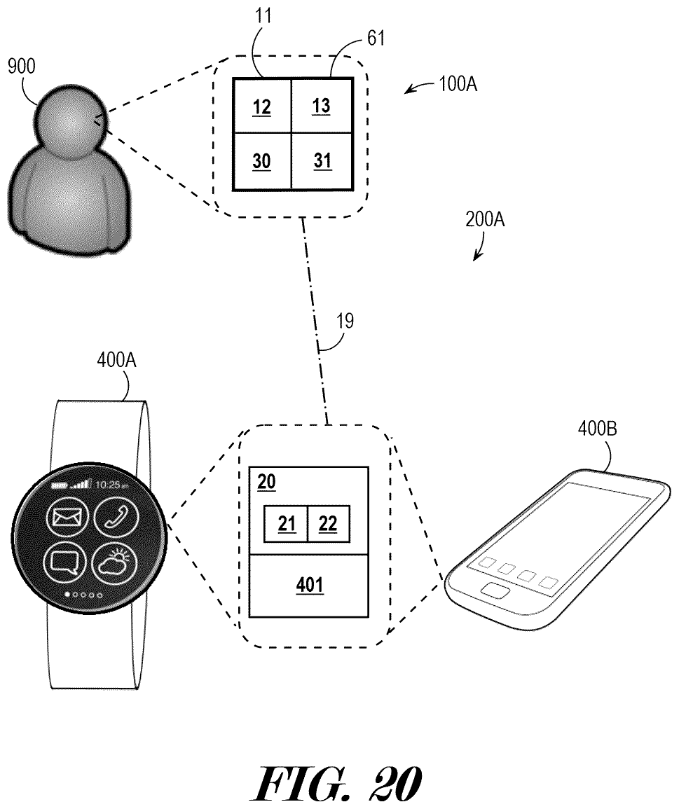

FIG. 21-FIG. 21 shows a schematic diagram of some exemplary components of another auricular automatic anti-arrhythmia system, having a dual-ear auricular ECG system and two taVNS units, according to various embodiments described herein.

FIG. 22-FIG. 22 illustrates a block diagram illustrating some exemplary components of an ear-finger ECG system according to various embodiments described herein.

FIG. 23-FIG. 23 illustrates a block diagram illustrating some exemplary components of a finger-finger ECG system according to various embodiments described herein.

FIG. 24-FIG. 24 shows a schematic diagram of some exemplary components of an ear-finger automatic anti-arrhythmia system, having an ear-finger ECG system and a taVNS unit, according to various embodiments described herein.

FIG. 25-FIG. 25 shows a schematic diagram of some exemplary components of a finger-finger automatic anti-arrhythmia system, having a finger-finger ECG system and a taVNS unit, according to various embodiments described herein.

FIG. 26-FIG. 26 shows a schematic diagram of some exemplary components of an ear-toe automatic anti-arrhythmia system, having an ear-toe ECG system and a taVNS unit, according to various embodiments described herein.

FIG. 27-FIG. 27 shows a schematic diagram of some exemplary components of a finger-toe automatic anti-arrhythmia system, having a finger-toe ECG system and a taVNS unit, according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

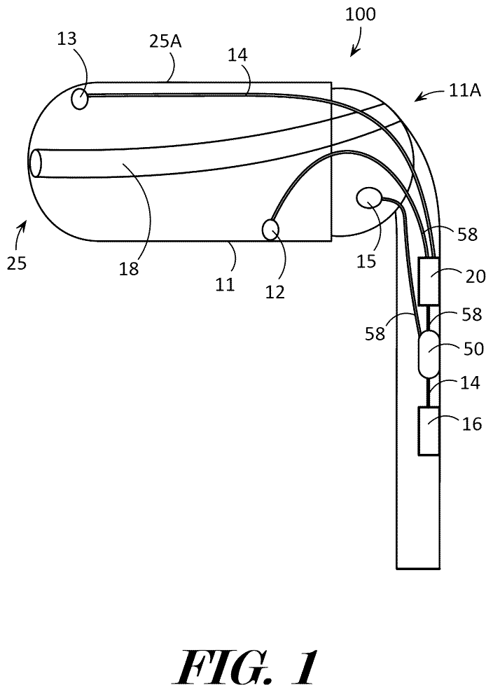

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, servers, laptops, tablet PCs including the iPad, cell phones including iOS phones made by Apple Inc., Android OS phones, Microsoft OS phones, Blackberry phones, Apple iPads, Anoto digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e., a “wireless network”) which may include BLE (Bluetooth), LoRa and LoRaWAN (and other low-power, wide-area (LPWA) networking protocols), Wi-Fi, and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigbee network, or a voice-over-IP (VOIP) network.

Although the terms “first”, “second”, “X” item, “Y” item etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 15% of the specified number. Additionally, as used in this application, the term “substantially” means that the actual value is within about 10% of the actual desired value, particularly within about 5% of the actual desired value and especially within about 1% of the actual desired value of any variable, element or limit set forth herein. Novel electrocardiogram (ECG) monitoring systems and novel automatic anti-arrhythmia systems are discussed herein. In the following description, for purposes of explanation, numerous specific details are set forth to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the figures or description below.

The present invention will now be described by example and through referencing the appended figures representing preferred and alternative embodiments. According to some embodiments consistent with the principles of the present invention, an auricular electrocardiogram (ECG) monitoring system (“the auricular ECG monitoring system”) 100 is disclosed. In some embodiments, the auricular electrocardiogram (ECG) monitoring system 100 may be configured as a single-ear auricular ECG system 100A. The single-ear auricular ECG system 100A may comprise an ECG recording module 20 that may comprise at least two wired or wireless ECG electrodes 12, 13, (ECG sensor electrodes 12, 13) (for example a first auricular ECG electrode 12 and a second auricular ECG electrode 13) in electronic communication with the ECG recording module 20. The auricular ECG electrodes 12, 13, may be in electronic communication with the ECG recording module 20 via a wire 14 or wirelessly. The wire 14 may comprise a wired connection type of local interface 58. The auricular ECG electrodes 12, 13, may be configured to be coupled to a wearer's first ear 902 or a peri-auricular area 903 around the wearer's first ear 902. When the auricular ECG electrodes 12, 13, are coupled to the wearer's first ear 902 or the peri-auricular area 903 around the wearer's first ear 902 the auricular ECG electrodes 12, 13, are positioned in contact with separate skin locations on at least one area of the wearer 900, the area selected from at least one of the following: external ear of the wearer's first ear 902, external ear canal 904 of the wearer's first ear, and the peri-auricular area 903 around the wearer's first ear. (It should be noted that the first and the second auricular ECG electrodes 12, 13 may contact separate skin locations within a single area. Alternatively, the first auricular ECG electrode 12 may contact a skin location within one area and the second auricular ECG electrode 13 may contact a separate skin location within another area.) These skin locations that the auricular ECG electrodes 12, 13, are attached to may be adequately separated (with different angles or directions) relative to the location of the heart of the wearer 900 so that they can pick up some differences of the cardiac action potentials in order to enable the ECG recording module 20 to perform an ECG. The ECG recording module 20 may be configured to record ECG data of the wearer 90 via the auricular ECG electrodes 12, 13, and the ECG recording module 20 may be in electronic communication with a processing unit 50, 401, which may be configured to record and analyze the ECG data from the ECG recording module 20 to detect presence or cessation of a serious cardiac arrhythmia. The processing unit 50, 401, may be further configured to record and analyze the ECG data from the ECG recording module 20 to detect presence or cessation of an impending serious cardiac arrhythmia. The serious cardiac arrhythmia may include atrial fibrillation (AFib or AF), atrial flutter (AFL), supraventricular tachycardia (SVT), ventricular tachycardia (VT), and ventricular fibrillation (VF) (Note: Wolff-Parkinson-White (WPW) syndrome is a serious cardiac arrhythmia that may be detected by ECG but not detected literally as an impending arrhythmia). As compared with a dual-ear auricular ECG system 100B (to be discussed hereinafter), the single-ear auricular ECG system 100A may have the advantage of being more convenient and more suitable for continuous monitoring although the ECG signals may be weaker than those of the dual-ear auricular ECG system 100B.

In some embodiments, the auricular electrocardiogram (ECG) monitoring system 100 may be configured as a dual-ear auricular ECG system 100B. The dual-ear auricular ECG system 100B may comprise an ECG recording module 20. The ECG recording module 20 may comprise at least two wired or wireless ECG electrodes 12, 88, in electronic communication with the ECG recording module 20. At least one of the at least two wired or wireless ECG electrodes 12, 88, (for example a first auricular ECG electrode 12) may be configured to be coupled to a wearer's first ear 902 or a peri-auricular area 903 around the wearer's first ear 902. When the first auricular ECG electrode 12 is coupled to the wearer's first ear 902 or the peri-auricular area 903 around the wearer's first ear the first auricular ECG electrode 12 is positioned in contact with an area of the wearer's skin, the area selected from at least one of the following: external ear 902 of the wearer's first ear, external ear canal 904 of the wearer's first ear, and the peri-auricular area 903 around the wearer's first ear. At least another one of the at least two wired or wireless ECG electrodes 12, 88, (for example a contralateral auricular ECG electrode 88) may be configured to be coupled to the wearer's second ear 902 or a peri-auricular area 903 around the wearer's second ear 902. When the contralateral auricular ECG electrode 88 is coupled to the wearer's second ear 902 or the peri-auricular area 903 around the wearer's second ear the contralateral auricular ECG electrode 88 is positioned in contact with an area of the wearer's skin, the area selected from at least one of the following: external ear 902 of the wearer's second ear, external ear canal 904 of the wearer's second ear, and the peri-auricular area 903 around the wearer's second ear. Preferably, the first auricular ECG electrode 12 and the contralateral auricular ECG electrode 88 are configured as wireless ECG electrodes and are in wireless communication with the ECG recording module 20. The ECG recording module 20 may comprise a wireless amplifier of an amplifier and filter 21. The ECG recording module 20 may be configured to record ECG data of the wearer via the wireless auricular ECG electrodes 12, 88 (including the wireless first auricular ECG electrode 12 and the wireless contralateral auricular ECG electrode 88) and the ECG recording module 20 may be in electronic communication with a processing unit 50, 401, which may be configured to record and analyze the ECG data from the ECG recording module 20 to obtain the wearer's ECG profile. The processing unit 50, 401 may be configured to analyze ECG data recorded by the ECG recording module 20 to detect presence or cessation of a serious cardiac arrhythmia of the wearer 900. The processing unit 50, 401 may be further configured to analyze ECG data recorded by the ECG recording module 20 to detect presence or cessation of an impending serious cardiac arrhythmia of the wearer 900. The serious cardiac arrhythmia may include atrial fibrillation (AFib or AF), atrial flutter (AFL), supraventricular tachycardia (SVT), ventricular tachycardia (VT), and ventricular fibrillation (VF). Preferably, the dural-ear auricular ECG system 100B may use wireless electronic communication although wired communication through wires in a headband could be an option. As compared with a single-ear auricular ECG system 100A, the dual-ear auricular ECG system 100B may have the advantage of stronger ECG signals and less interference from artifacts although the dual-ear auricular ECG system 100B might be less convenient for users.

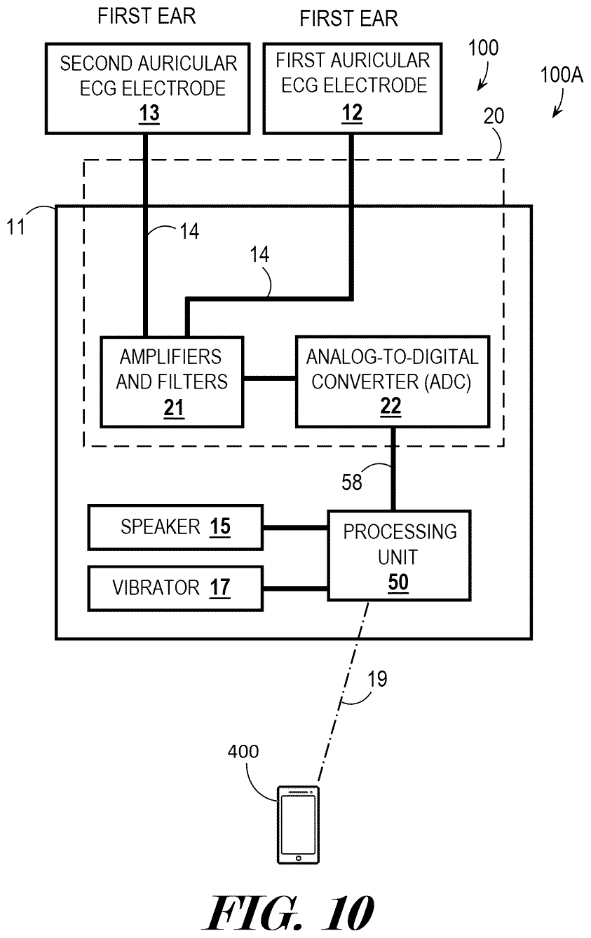

The auricular ECG monitoring system 100 may comprise an ECG recording module 20. The ECG recording module 20 may record and analyze the electrical activity of the wearer's 900 heart, such as to diagnose heart conditions and cardiac arrhythmias. Referring to FIGS. 10 &11, amplifiers and filters 21 may pick up the electrical activity of the wearer's 900 heart via the ECG electrodes 12, 13, 88. An amplifier of amplifiers and filters 21 is responsible for amplifying the weak electrical signals received from the auricular ECG electrodes 12, 13, 88. The heart's electrical signals are typically very faint (for example, in the range of 10 microvolts to 5 millivolts, depending on chest or limb leads and depending on different waves including P, QRS or T waves). An amplifier of amplifiers and filters 21 boosts these signals to a level that can be accurately recorded and displayed. Modern ECG machines use sophisticated amplifiers that minimize noise and ensure signal clarity. Filters of amplifiers and filters 21 are used to remove unwanted noise and interference from the electrical signals. Common sources of noise include muscle contractions, electrical interference from other devices, and movement artifacts. ECG machines use various filters, such as high-pass, low-pass, and notch filters, to clean the signals, ensuring that the resulting ECG trace is clear and interpretable. An analog-to-digital converter (ADC) 22 may transform the analog electrical signals from the heart into digital data. This digital conversion is essential for processing, storing, and/or displaying the ECG data on a display screen 404C or print out. The ADC 22 ensures that the data is accurately digitized, preserving the integrity of the original signal. The ADC 22 may be in communication with a processing unit 50, 401.

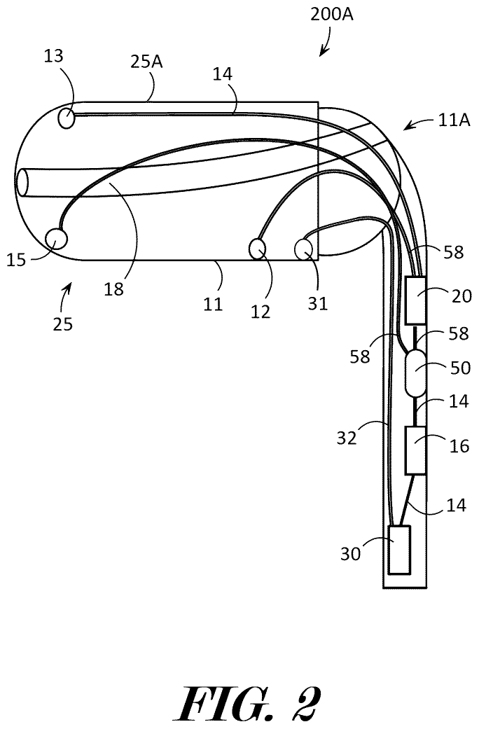

In some embodiments, an auricular ECG monitoring system 100 may comprise an ECG recording module 20 that may be contained in an auricular housing structure 11. In some embodiments, a single-ear auricular ECG system 100A of the present invention may comprise an auricular housing structure 11 which may be configured to be coupled to a first ear 902 of a wearer 900 and/or to a peri-auricular area 903 around the first ear 902 of the wearer 900 and the auricular housing structure 11 may be configured to house all of the ECG electrodes 12, 13. In some embodiments, a dual-ear auricular ECG system 100B of the present invention may comprise two auricular housing structures 11 and one of the auricular housing structure 11 may be configured to be coupled to a first ear 902 of a wearer 900 and/or to the peri-auricular area 903 around the first ear 902 of the wearer 900 and be configured to house the first auricular ECG electrode 12, while another auricular housing structure 11 may be configured to be coupled to a second ear 902 of the wearer 900 and/or to a peri-auricular area 903 around the second ear 902 of the wearer 900 and be configured to house the contralateral auricular ECG electrode 88. In some embodiments, the one or two auricular housing structure(s) 11 may house one or more components, such as a processing unit 50, an ECG recording module 20, auricular ECG electrodes 12, 13, a contralateral auricular ECG electrode 88, a speaker 15, a power source 16, etc., which may be communicatively coupled via a local interface 58. The local interface 58 can be, for example but not limited to, one or more buses, circuit boards, wiring harnesses, or other wired connections or wireless connections, as is known in the art. The local interface 58 can have additional elements, which are omitted for simplicity, such as controllers, buffers (caches), drivers, repeaters, and receivers, among many others, to enable communications. Further, the local interface 58 may include address, control, and/or data connections to enable appropriate communications among the aforementioned components. In some embodiments, an auricular housing structure 11 may comprise as one of: an earbud style structure 11A, a tubular-shaped structure 11B, or a behind-the-ear-hearing-aid-style structure 11C. In some embodiments, an auricular housing structure 11, such as an earbud style structure 11A, may comprise a sound conduit 18 which may extend through the auricular housing structure 11 (such as shown in FIGS. 1, 2) and which may facilitate the ability of sound to enter the ear 902. Preferably, a sound conduit 18 may comprise an opening, channel, conduit, etc., which may extend through a portion of an earbud style structure 11A so that sound waves may pass through the sound conduit 18 to facilitate or enable the wearer 900 to hear sounds in the environment.

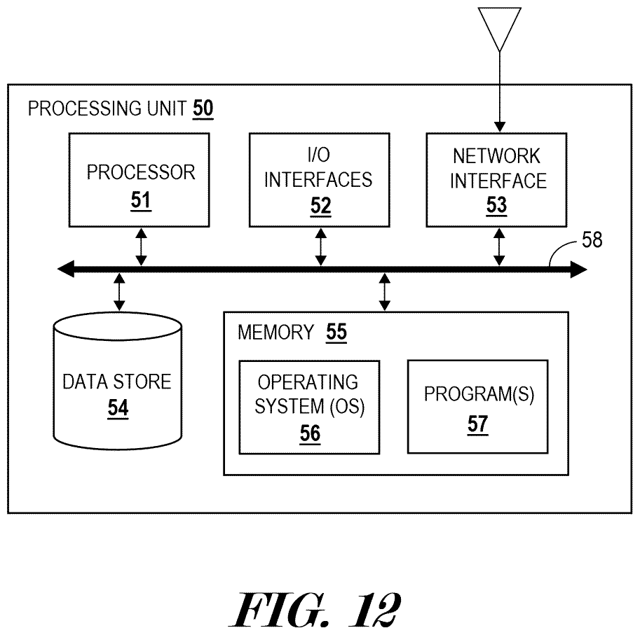

In some embodiments, an auricular ECG monitoring system 100 may comprise a processing unit 50. Referring to FIG. 12, the processing unit 50 may comprise a memory 55 that may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 55 may incorporate electronic, magnetic, optical, and/or other types of storage media. Note that the memory 55 may have a distributed architecture, where various components are situated remotely from one another, but can be accessed by the processor 51. Optionally, memory 55 can include one or more software programs, each of which includes an ordered listing of executable instructions for implementing logical functions. Optionally, the software in the memory system 55 includes a suitable operating system (O/S) 56 and program(s) 57. The operating system 56 essentially controls the execution of input/output interface 52 and other element functions, and provides scheduling, input-output control, file and data management, memory management, and communication control and related services. The operating system 56 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 57 may include various applications, add-ons, etc. configured to provide end user functionality of the device 100.

In some embodiments, a processing unit 50 may comprise one or more I/O interfaces 52 which can be used to provide user input and display system output data, such as operational status. The I/O interfaces 52 can include, for example, buttons, knobs, switches, LED indicator lights, LED display, LCD display, a serial port, a parallel port, a small computer system interface (SCSI), an infrared (IR) interface, a radio frequency (RF) interface, a universal serial bus (USB) interface, and the like. Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

In some embodiments, an auricular ECG monitoring system 100 may comprise a network interface 53 which may be contained in the auricular housing structure 11 and which may enable wired and/or wireless communication between one or more components, such as ECG recording module 20, processing unit 50, etc., with one or more client devices 400. Preferably, a network interface 53 may comprise a radio that may operate via WiFi and/or Bluetooth communication standards. In further embodiments, a network interface 53 may comprise a radio that may operate on a cellular band and may communicate with or receive a Subscriber Identity Module (SIM) card or other wireless network identifier. Any number of suitable wireless data communication protocols, techniques, or methodologies can be supported by a network interface 53, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Near-Field Communication (NFC); Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. In further embodiments, a network interface 53 may enable wired network communication and may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 53 may include address, control, and/or data connections to enable appropriate communications on the network.

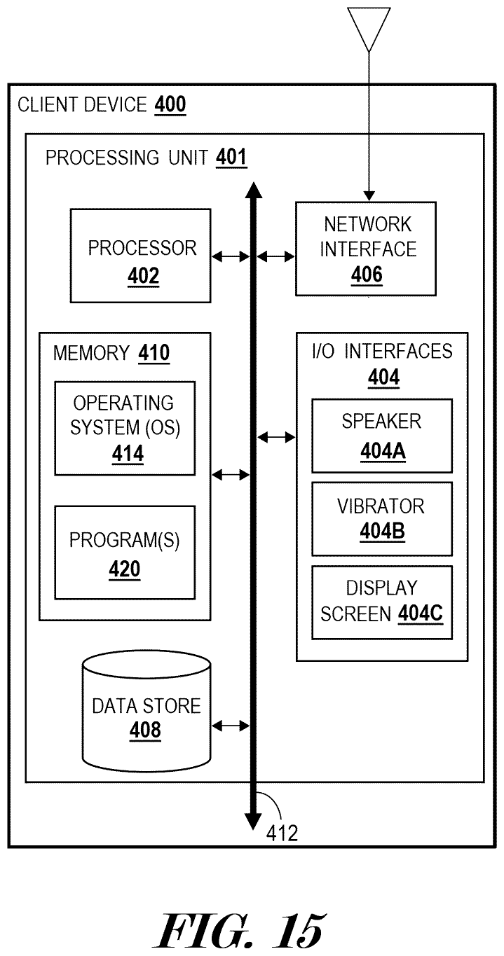

In some embodiments, the auricular ECG monitoring system 100 may be in communication with one or more client devices 400. In some embodiments, the auricular ECG monitoring system 100 may comprise one or more client devices 400. Referring to FIG. 15, in an exemplary embodiment, a block diagram illustrates a client device 400 of which may be a type of computing platform. A client device 400 can be a digital device that, in terms of hardware architecture, generally includes a processing unit 401 with a processor 402, input/output (I/O) interfaces 404, a network interface 406, a data store 408, and memory 410. It should be appreciated by those of ordinary skill in the art that FIG. 15 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications. The I/O interfaces 404 can be used to receive data and user input and/or for providing system output. User input can be provided via a plurality of I/O interfaces 404, such as a keypad, a touch screen, a speaker 404A, a vibrator 404B, a camera, a microphone, a scroll ball, a scroll bar, buttons, barcode scanner, voice recognition, eye gesture, and the like. System output can be provided via a display screen 404C such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker 404A configured to emit audio notifications, a vibrational device or vibrator 404B configured to vibrate, shake, or produce any other series of rapid and repeated movements to produce haptic notifications, and/or a light emitting diode (LED) or other light emitting element which may be configured to illuminate to provide a visual notification.

In some embodiments, the auricular ECG monitoring system 100 may comprise a network interface 53, 406, and the network interface 53, 406, may be configured to generate an electronic notification via an input/output (I/O) interface 404 of a client device 400 when the processing unit 50, 401, detects the presence or cessation of a serious cardiac arrhythmia or an impending serious cardiac arrhythmia of the wearer 900 of the auricular ECG monitoring system 100. In preferred embodiments, the auricular ECG monitoring system 100 may comprise one or more client devices 400 that may be configured to generate a notification when a processing unit 50, 401, detects the presence or cessation of a serious cardiac arrhythmia or an impending serious cardiac arrhythmia. For example, when a serious cardiac arrhythmia or an impending serious cardiac arrhythmia is detected by a processing unit 50, a network interface 53 of the processing unit 50 may send a notification to the client device 400 of the wearer 900 of the auricular ECG monitoring system 100 and/or may send a notification to the client device 400 of the wearer's healthcare provider 950 to take appropriate actions. When cessation of serious cardiac arrhythmia or cessation of the impending serious cardiac arrhythmia is detected by the processing unit 50 in the ECG recording housing 11, the network interface 53 of the processing unit 50 may send a notification to the client device 400 of the wearer 900 of the auricular ECG monitoring system 100 and/or may send a notification to the client device 400 of the wearer's healthcare provider 950 to take appropriate actions. Similar functions may be provided by a processing unit 401 located on a client device 400. As an alternate example, when a serious cardiac arrhythmia or an impending serious cardiac arrhythmia is detected by a processing unit 401 of a client device 400 that is in electronic communication 19 with the ECG recording module 20 (via a network interface 53), the processing unit 401 of the client device 400 of the wearer 900 may generate a notification and/or a client device 400 of the wearer's healthcare provider 950 may generate a notification to enable the wearer's healthcare provider 950 to take appropriate actions. When cessation of serious cardiac arrhythmia or impending serious cardiac arrhythmia is detected by the processing unit 401 of the client device 400 that is in electronic communication 19 with the ECG recording module 20 (via a network interface 53), the processing unit 401 of the client device 400 of the wearer 900 of the ECG recording housing 11 may generate a notification and/or a client device 400 of the wearer's healthcare provider 950 may generate a notification to enable the wearer's healthcare provider 950 to take appropriate actions.

In some embodiments, the auricular ECG monitoring system 100 may comprise a data store 408. The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media. The memory 410 may include any of volatile memory elements (e.g., random access memory (RAM, such as DRAM, SRAM, SDRAM, etc.)), and nonvolatile memory elements (e.g., ROM, hard drive, etc.). Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs 420, each of which includes an ordered listing of executable instructions for implementing logical functions.

The ECG recording module 20 may be configured to record ECG data of the wearer 900. The auricular ECG monitoring system 100 is in electronic communication with a processing unit 50, 401, that is configured to detect presence or cessation of serious cardiac arrhythmias and presence or cessation of impending serious cardiac arrhythmia, with the help of various ECG analysis algorithms (as known in the art). Recent advances in ECG analysis algorithms have helped detection of impending serious cardiac arrhythmia. Key predictive patterns of premature atrial contractions (PACs) have been found to help predict impending development of paroxysmal atrial fibrillation (AFib), including frequency of PACs (or PAC burden) (for example more than 3,000 PACs in a 24-hour period). Origin of PAC from pulmonary veins area is also a highly critical predictor. The pattern (clustering and timing) of PACs, including short rapid bursts or clusters of PACs, using clustering analysis algorithms and variable timing analysis algorithms can also help to predict paroxysmal atrial fibrillation. With artificial intelligence and machine learning, it has been shown that clustering PACs based on their P-wave morphology, timing, and coupling intervals can predict impending AFib. A high variability in the timing of PACs, especially in the coupling interval (the time between the premature beat and the preceding normal beat), has also been found to help predict impending AFib risk. Presence of these specific PACs patterns indicate atrial cardiomyopathy that make the patient vulnerable to atrial fibrillation.

Similarly, key predictive patterns of premature atrial contractions (PACs) have been found to help predict development of atrial flutter (AFL). These include frequency of PACs (PAC burden) (for example over 3,000 PACs in 24 hours). The temporal patterns of PACs are also correlated with risk of impending atrial flutter. With artificial intelligence and machine learning to analyze ECG data, distinct clusters (high-risk clusters) of PACs have been identified, including the PACs morphology, timing, and coupling interval and these have been developed into ECG analysis algorithms for prediction of impending AFL. Source of the PACs (from pulmonary vein area) also helps in prediction of impending atrial flutter.

Likewise, key predictive patterns of premature atrial contractions (PACs) have been found to help predict development of supraventricular tachycardia (SVT). Frequent PACs (for example: over 3,000 PACs in 24 hours) is again indicative of high risk for SVT. The PAC patterns can also help to predict SVT, including repetitive and organized PAC patterns significantly increase the risk of SVT as compared to simple, isolated PACs, including bigeminy and trigeminy. Occasional short run of non-sustained SVT can also predict impending sustained SVT. Early PACs that are blocked (not conducted to the ventricles) can also be a significant risk factor for impending more complex SVT.

There are ECG patterns that can help to predict impending ventricular tachycardia (VT). VT may occur with underlying cardiac diseases, for example Brugada syndrome with its specific ECG patterns (prominent, coved ST-segment elevation of at least 2 mm in leads V1 and V2 with an associated T-wave inversion.) or arrhythmogenic right ventricular cardiomyopathy (ECG showing T-wave inversion, Epsilon wave, prolong S-wave upstroke). Other ECG technics, like signal-averaged ECG and heart-rate variability may also help to predict impending ventricular tachycardia (VT). Frequent premature ventricular contractions (PVCs) or high burden of PVCs, especially with uniform morphology, can help to predict impending VT. Algorithms for differentiating VT from other wide-complex tachycardias are known in the art.

Similarly, there are ECG patterns and features that can help predict impending ventricular fibrillation (VF). These ECG markers may include T-wave alternans, prolonged T-peak to T-end interval, QT interval prolongation, early repolarization pattern, fragmented QRS, long QRS duration, Epsilon waves, frequent PVCs or episodes of non-sustained ventricular tachycardia, etc. and presence of these may indicate increased risks of impending ventricular fibrillation.

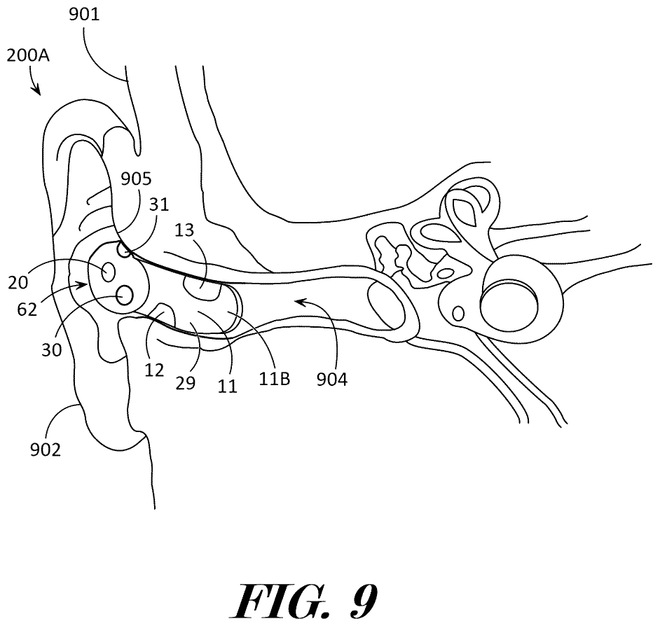

According to another aspect consistent with the principles of the present invention, an auricular automatic anti-arrhythmia system 200A is disclosed (FIGS. 2, 7, 8, 9). In some embodiments, an auricular automatic anti-arrhythmia system 200A may comprise an auricular electrocardiogram (ECG) monitoring system 100 having an ECG recording module 20, such as described hereinbefore, that may be configured to record ECG data of the wearer 900. The auricular ECG monitoring system 100 is in electronic communication with a processing unit 50, 401, that is configured to detect presence or cessation of serious cardiac arrhythmias and presence or cessation of impending serious cardiac arrhythmias with the help of various ECG analysis algorithms (as known in the art). Serious cardiac arrhythmias may include atrial fibrillation (AFib), atrial flutter (AFL), supraventricular tachycardia (SVT), ventricular tachycardia (VT), and ventricular fibrillation (VF). The auricular automatic anti-arrhythmia system 200A may further comprise a transcutaneous auricular vagus nerve stimulation (taVNS) unit 30. The taVNS unit 30 comprises a miniature stimulating electrode 31 that is configured to contact or be attached to vagus nerve innervated auricular skin of the wearer 900. The vagus innervated auricular skin includes external ear canal 904, tragus 905, cymba-concha 906, cavum-concha 907 and small adjacent areas. The vagus innervated auricular skin that the stimulating electrode 31 is configured to contact may be selected from at least one of the following: external ear canal 904, tragus 905, cymba-concha 906, and cavum-concha 907 of the wearer's ear 902. When prompted (by signals from the processing unit 50, 401) the taVNS unit 30 is configured to generate electric stimulation to the vagus innervated auricular skin via the stimulating electrode 31. The ECG recording module 20 may be in wired or wireless electronic communication (e.g., through wire, Bluetooth, etc.) with a processing unit 50, 401. The taVNS unit 30 may also be in electronic communication with the processing unit 50, 401. The processing unit 50, 401, may be configured to analyze ECG data recorded by the auricular ECG monitoring system 100 to detect the presence or cessation of ECG signals suggestive of serious cardiac arrhythmias or impending serious cardiac arrhythmia. When the presence of ECG signals suggestive of at least one serious cardia arrhythmia is detected by the processing unit 50, 401, the processing unit 50, 401, may be configured to immediately send signals (arrhythmia-warning signals) to the taVNS unit 30. Upon receiving arrhythmia-warning signals from the processing unit 50, 401, the taVNS unit 30 is configured to automatically start sending pre-determined electric stimuli to the vagus innervated auricular skin of the wearer's ear 902 to which the stimulating electrode 31 is in contact with. (Pre-determined stimulating parameters of taVNS 30 for serious cardiac arrhythmia as shown in Table 1). When cessation of ECG signals suggestive of the at least one serious cardiac arrhythmia is detected by the processing unit 50, 401, the processing unit 50, 401, may be further configured to immediately send signals (arrhythmia-cessation signals) to the taVNS unit 30. Upon receiving arrhythmia-cessation signals from the processing unit 50, 401, the taVNS unit 30 is configured to automatically stop sending pre-determined electric stimuli to the vagus innervated auricular skin of the wearer's ear 902 to which the stimulating electrode 31 is in contact with. When the presence of ECG signals suggestive of at least one impending serious cardia arrhythmia is detected by the processing unit 50, 401, the processing unit 50, 401, may be configured to immediately send signals (impending-arrhythmia-warning signals) to the taVNS unit 30. Upon receiving impending-arrhythmia-warning signals from the processing unit 50, 401, the taVNS unit 30 is configured to automatically start sending pre-determined electric stimuli to the vagus innervated auricular skin of the wearer's ear 902 to which the stimulating electrode 31 is in contact with. (Pre-determined stimulating parameters of taVNS 30 for impending serious cardiac arrhythmia as shown in Table 2). When cessation of ECG signals suggestive of the at least one impending serious cardiac arrhythmia is detected by the processing unit 50, 401, the processing unit 50, 401, may be further configured to immediately send signals (impending-arrhythmia-cessation signals) to the taVNS unit 30. Upon receiving impending-arrhythmia-cessation signals from the processing unit 50, 401, the taVNS unit 30 is configured to automatically stop sending pre-determined electric stimuli to the vagus innervated auricular skin of the wearer's ear 902 to which the stimulating electrode 31 is in contact with.

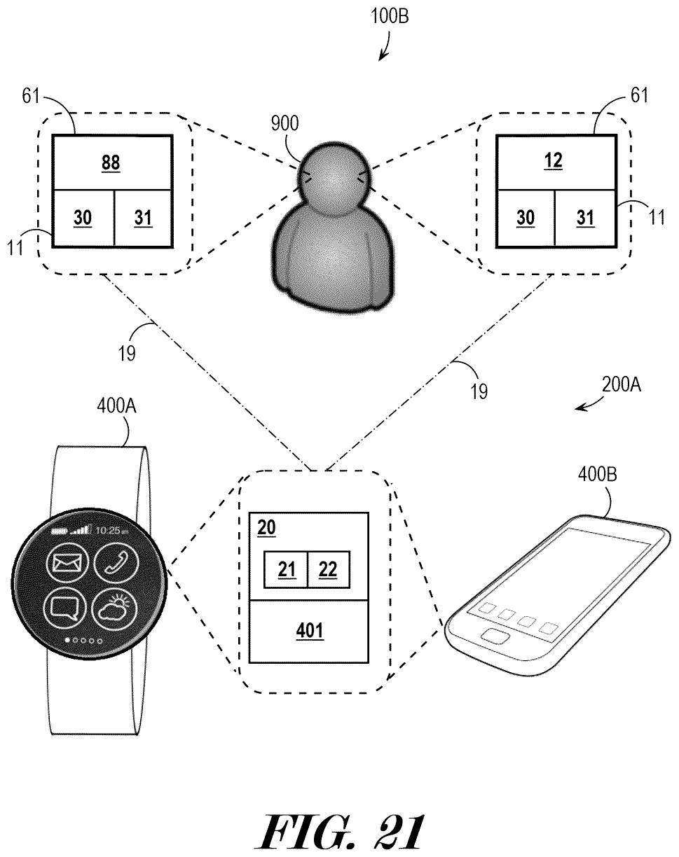

In some embodiments, an auricular automatic anti-arrhythmia system 200A may comprise one or two transcutaneous auricular vagus nerve stimulation (taVNS) units 30. Optionally, an auricular automatic anti-arrhythmia system 200A may comprise a first taVNS unit 30 and a second taVNS unit 30. (FIG. 21). The first taVNS unit 30 comprises a first stimulating electrode 31 that is configured to contact vagus innervated auricular skin of the wearer's first ear 902; while the second taVNS unit 30 has a second stimulating electrode 31 that is configured to contact vagus innervated auricular skin of the wearer's second ear 902. (It had been reported that bilateral vagus nerve stimulation with bilateral taVNS units 30 might be more effective than unilateral vagus nerve stimulation). A processing unit 50, 401, may be in electronic communication with the first and the second taVNS units 30. When the presence of ECG signals suggestive of a serious cardiac arrhythmia is detected by the processing unit 50, 401, the processing unit 50, 401, may be configured to immediately send signals (arrhythmia-warning signals) to the first and the second taVNS units 30 to cause the first and the second taVNS units 30 to automatically start sending pre-determined electric stimuli to the vagus innervated auricular skin of the wearer's first and second ears 902. When cessation of ECG signals suggestive of serious cardiac arrhythmia is detected by the processing unit 50, 401, the processing unit 50, 401, may be further configured to immediately send signals (arrhythmia-cessation signals) to the first and the second ta VNS units 30 to cause the first and the second taVNS units 30 to automatically stop sending pre-determined electric stimuli to the vagus innervated auricular skin of the wearer's first and second ears 902. Likewise, when the presence of ECG signals suggestive of an impending serious cardiac arrhythmia is detected by the processing unit 50, 401, the processing unit 50, 401, may be configured to immediately send signals (impending-arrhythmia-warning signals) to the first and the second taVNS units 30 to cause the first and the second taVNS units 30 to automatically start sending pre-determined electric stimuli to the vagus innervated auricular skin of the wearer's first and second ears 902. When cessation of ECG signals suggestive of impending serious cardiac arrhythmia is detected by the processing unit 50, 401, the processing unit 50, 401, may be further configured to immediately send signals (impending-arrhythmia-cessation signals) to the first and the second taVNS units 30 to cause the first and the second taVNS units 30 to automatically stop sending pre-determined electric stimuli to the vagus innervated auricular skin of the wearer's first and second ears 902.

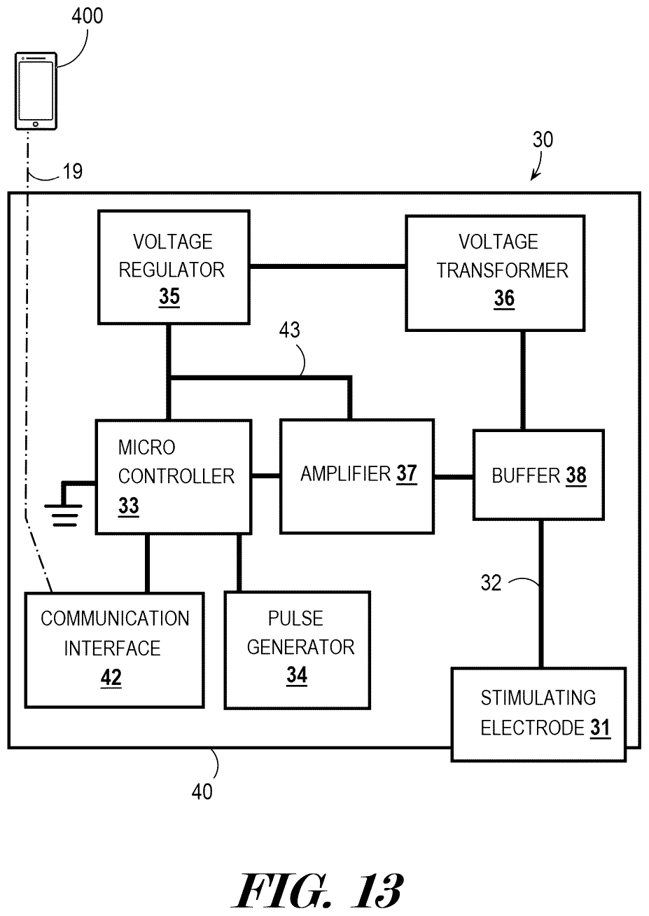

In a preferred embodiment, an auricular automatic anti-arrhythmia system 200A may comprise a transcutaneous auricular vagus nerve stimulation (taVNS) unit 30 and an auricular ECG monitoring system 100. A taVNS unit 30 may comprise any device that is able to provide transcutaneous auricular vagus nerve stimulation to a user's body. As an example, and referring to FIG. 13, a taVNS unit 30 may comprise a microcontroller 33 that may be in communication with a pulse generator 34, voltage regulator 35, voltage transformer 36, amplifier 37, and buffer 38, and that may be configured to generate taVNS electric stimuli that may be delivered to vagus nerve innervated auricular skin via a stimulating electrode 31. Optionally, a taVNS unit 30 may comprise a communication interface 42 which may enable electronic communication 19 (e.g., wired and/or wireless communication) between the taVNS unit 30 and another electronic device, such as a client device 400, an auricular ECG monitoring system 100, etc. Preferably, a communication interface 42 may comprise a radio that may operate via WiFi and/or Bluetooth communication standards. In some embodiments, a communication interface 42 may be configured as a network interface 53 described above so that it may operate on any wireless and/or wired electronic communication 19 protocol that a network interface 53 may use.

One or more components (31, 33, 34, 35, 36, 37, 38, 42) of a taVNS unit 30 may be contained in a taVNS housing 40 and may be in electronic communication via a local interface 43. A taVNS housing 40 may be configured in any size and shape, and may be made from or comprise soft foam, elastomer, soft silicone or any other material used in the fields of personal medical devices. In some embodiments, a taVNS housing 40 may be configured as one of: an earbud-style structure 11A, a tubular-shaped structure 11B or a behind-the-ear-hearing-aid-style structure 11C. Optionally, a taVNS housing 40 may be coupled directly to an auricular housing structure 11 for the auricular ECG electrodes 12, 13, of an auricular ECG monitoring system 100. In some embodiments, the taVNS housing 40 and the auricular housing structure 11 for auricular ECG electrodes 12, 13, may be integrally formed or molded together as a single unit. Optionally, a taVNS housing structure 40 may be a standalone housing that may be remote from an auricular housing structure 11 for auricular ECG electrodes 12, 13. In some embodiments, the taVNS unit 30 may include a stimulating electrode 31 which may be built within the taVNS housing 40 of the taVNS unit 30 (so that the taVNS housing 40 and stimulating electrode 31 may form a single unit). In further embodiments, a stimulating electrode 31 may be connected with the taVNS housing through a wire (so that the stimulating electrode 31 may be remote from the taVNS housing of the taVNS unit 30). The stimulating electrode 31 of a taVNS unit 30 may be attached to vagus nerve innervated auricular skin so that the stimuli output by the taVNS unit 30 may be delivered to the vagus nerve innervated auricular skin via the stimulating electrode 31. Vagus nerve innervated auricular skin may be selected from one of the following: tragus 905, cymba-concha 906, cavum-concha 907, and external ear canal 904. Optionally, the ta VNS unit 30 itself may also be attached to the tragus 905, cymba-concha 906, cavum-concha 907, or external ear canal 904. The tragus 905, concha region 906, 907, and external ear canal 904 are inherently stable for attachment of a taVNS unit 30 on a long-term basis.

When ECG signals suggestive of a serious cardiac arrhythmia is detected, the processing unit 50, 401, may be configured to automatically send signals (arrhythmia-warning signals) immediately to the one or more taVNS units 30 of the auricular automatic anti-arrhythmia system 200A to actuate the vagus nerve stimulation via stimulating electrode(s) 31, utilizing pre-determined stimulation parameters, such as shown in Table 1.

TABLE 1
Example of taVNS unit 30 electric stimuli output parameters for
a serious cardiac arrhythmia:

| Output | Parameter |
|---|---|
| Power supply | Direct current 3-9 volts |
| Pulse width | 100-1000 microseconds, more preferably |
| | 0.25-1 millisecond (ms) |
| Frequency | 0.5-200 Hz, more preferably 20-25 |
| | Hertz (Hz) |
| Modes | Continuous wave or sparse-dense wave |
| Intensity | 0.1-15 milliamperes (mA), more preferably |
| | 0.2-2 mA |

When ECG signals suggestive of an impending serious cardiac arrhythmia is detected, the processing unit 50, 401, may be configured to automatically send signals (impending-arrhythmia-warning signals) immediately to the one or more taVNS units 30 of the auricular automatic anti-arrhythmia system 200A to actuate the vagus nerve stimulation via stimulating electrode(s) 31, utilizing pre-determined stimulation parameters, such as shown in Table 2.

TABLE 2
Example of taVNS unit 30 electric stimuli output parameters for
an impending serious cardiac arrhythmia:

| Output | Parameter |
|---|---|
| Power supply | Direct current 3-9 volts |
| Pulse width | 100-800 microseconds, more preferably |
| | 0.2-0.7 millisecond (ms) |
| Frequency | 0.5-100 Hz, more preferably 10-20 |
| | Hertz (Hz) |
| Modes | Continuous wave or sparse-dense wave |
| Intensity | 0.1-15 milliamperes (mA), more preferably |
| | 0.15-1.5 mA |

In some embodiments, an auricular automatic anti-arrhythmia system 200A may comprise an auricular ECG monitoring system 100 that is configured as a single-ear auricular ECG system 100A. The single-ear auricular ECG system 100A may comprise an ECG recording module 20 and at least two wired or wireless ECG electrodes 12, 13, including a first auricular ECG electrode 12 and a second auricular ECG electrode 13. The first auricular ECG electrode 12 and the second auricular ECG electrode 13 may be configured to be coupled to a first ear 902 of a wearer 900 or a peri-auricular area 903 around the wearer's first ear 902. The ECG recording module 20 is in electronic communication with each auricular ECG electrode 12, 13. When the first auricular ECG electrode 12 and the second auricular ECG electrode 13 are coupled to the wearer's first ear 902 or the peri-auricular area 903 around the wearer's first ear 902 the first auricular ECG electrode 12 and the second auricular ECG electrode 13 are positioned in contact with separate skin locations of at least one area of the wearer's skin, the at least one area of the wearer's skin selected from at least one of the following: an external ear 902 of the wearer's first ear, an external ear canal 904 of the wearer's first ear, and the peri-auricular area 903 around the wearer's first ear. (It should be noted that the first and the second auricular ECG electrodes 12, 13 may contact separate skin locations within a single area. Alternatively, the first auricular ECG electrode 12 may contact a skin location within one area and the second auricular ECG electrode 13 may contact a separate skin location within another area.) The ECG recording module 20 is configured to record ECG data of the wearer 900 via the at least two wired or wireless ECG electrodes 12, 13, including the first and second auricular ECG electrodes 12, 13.

In some embodiments, an auricular ECG monitoring system 100 may comprise an auricular housing structure 11 selected from one of the followings: an earbud-style structure 11A, a tubular-shaped structure 11B and a behind-the-ear-hearing-aid-style structure 11C. In some embodiments, a single-ear auricular ECG monitoring system 100A may comprise an auricular housing structure 11. The auricular housing structure 11 includes an in-the-ear portion 27 that can be placed into a wearer's external ear canal 904 and bowl-shaped area of tragus-concha 905, 906, 907.

As used herein, the term “auricular housing structure 11” refers to housing structures in or around the ear. Examples of auricular housing structures include an earbud-style structure 11A, a behind-the-ear-hearing-aid-style structure 11C, a tubular-shaped structure 11B and a taVNS housing 40 used for a taVNS unit 30. An auricular housing structure 11 may comprise as one of: an earbud style structure 11A, a tubular-shaped structure 11B, or a behind-the-ear-hearing-aid-style structure 11C. Also, as used herein, the term “in-the-ear portion 27” of an auricular housing structure 11 refers to a portion of the auricular housing structure 11 that is placed inside an external ear canal 904 and a bowl-shaped area of tragus-concha 905, 906, 907, when in use. As used herein, the term “bowl-shaped area of tragus-concha” 905, 906, 907, refers to an area surrounding the opening of the external ear canal 904 and this area is bounded by inner tragus 905 and concha 907, 906, forming a bowl-shaped structure. The “in-the-ear portion 27” of the auricular housing structure 11 is essentially the same as an in-the-ear portion 27 of a behind-the-ear-hearing-aid-style structure 11C (a behind-the-ear-hearing-aid-style structure 11C typically includes an in-the-ear portion 27 and a behind-the-ear portion 26.) Additionally, as used herein, the term “horizontal portion 25” of an earbud-style structure 11A refers to the “body”, “nozzle” and “ear-tip” of the earbud 11A and the “nozzle” and “ear-tip” are typically inserted inside an external ear canal 904 while the “body” is typically sitting in the bowl-shaped area of tragus-concha 905, 906, 907, when put in use. Thus, the term “horizontal portion 25” of the earbud-style structure 11A is essentially the same as the “in-the-ear portion 27” of an auricular housing structure 11. The tubular-shaped structure 11B itself is a housing structure located inside an external ear canal 904 and thus is essentially part of the “in-the-ear portion 27” of the auricular housing structure 11.

Additionally, as used herein, the terms “self-installable” or “self-removable” electrode refers to an electrode (including auricular ECG electrodes 12, 13, 86, 88, stimulating electrode 31 of a taVNS unit 30, finger-ring electrodes 83, 84, and toe-ring electrode 73) that can be installed or removed by an ordinary adult wearer (or user) himself or herself without any help from other person or from a technologist (no need for the technologist to prepare the skin surface and no need to use special adhesive material to secure the electrodes to the skin). The terms “self-installation” or “self-removal” of an electrode (12, 13, 86, 88, 31, 83, 84, 73) refer to installing or removing the electrode (12, 13, 86, 88, 31, 83, 84, 73) by an ordinary adult wearer or user himself/herself without any help from a technologist or other person. Likewise, the term “self-installable and self-removable” system refers to a system that uses self-installable and/or self-removable electrode. As used herein, all of the self-installable self-removable housing structures that are used to house ECG electrodes (for example auricular electrodes 12, 13, 88, 86), taVNS stimulating electrode 31, and smart ring ECG electrodes (including first finger-ring electrode 83, second finger-ring electrode 84 and first toe-ring electrode 73) in this invention can be referred collectively as “self-installable-housing structures” and these electrodes can be referred collectively as “self-installable electrodes”. Examples of self-installable-housing structures may include the in-the-ear portion 27 of the auricular housing structure 11, an earbud-style structures 11A, a hearing-aid-style structure 11B, a tubular-shaped structure 11C, and smart ring-type structures for fingers or toes (for example first finger-ring 83A, second finger-ring 84A, first toe-ring 73A). These self-installable housing structures can be self-installed and self-removed easily by the wearer (user) without any help from another person or a technologist and there is no need to use adhesive material to secure the electrodes. There is also no need to prepare the skin surface before attaching the electrode.

In some embodiments, a single-ear auricular ECG system 100A may comprise an auricular housing structure 11. The auricular housing structure 11 includes an in-the-ear portion 27 that can be placed into an external ear canal 904 and a bowl-shaped area of tragus-concha 905, 906, 907 of a wearer's ear 902 when in use. The single-ear auricular ECG system 100A comprises an ECG recording module 20. The ECG recording module 20 comprises at least two wired or wireless ECG electrodes 12, 13, including a first auricular ECG electrode 12 and a second auricular ECG electrode 13. The first and second auricular ECG electrodes 12, 13, may be configured to be housed in the in-the-ear portion 27 of the auricular housing structure 11 to be coupled to a first ear 902 of a wearer 900. The ECG recording module 20 may be configured to be housed in the auricular housing structure 11 or be housed in a client device 400 such as a wearable watch-type device 400A or a portable smart phone type device 400B. The first auricular ECG electrode 12 and the second auricular ECG electrode 13 may be configured to be partially embedded in a surface 27A of the in-the-ear portion 27 with slight protrusion at the surface 27A of the in-the-ear portion 27. The in-the-ear portion 27 may be configured to be comprised of (or made of) a flexible elastic and adaptable material (such as soft foam or soft silicone-type material) and the flexible elastic and adaptable material is configured to have appropriate flexibility, elasticity and adaptability such that the in-the-ear portion 27 will naturally adapt to the contour of the wearer's external ear canal 904 and the contour of the bowl-shaped area of tragus-concha 905, 906, 907, of the wearer's first ear 902, and snugly fill the interior of the external ear canal 904 and the bowl-shaped area of tragus-concha 905, 906, 907, of the wearer's first ear 902, when the in-the-ear portion 27 is put in place and such that the first auricular ECG electrode 12 and the second auricular ECG electrode 13 are naturally and snugly in contact with the skin of the external ear canal 904 or skin of bowl-shaped area of tragus-concha 905, 906, 907, of the wearer's first ear 902, when the in-the-ear portion 27 is put in place. This setup will make the first and second auricular ECG electrodes 12, 13, easily self-installable and self-removable since installing these auricular ECG electrodes 12, 13, will be as easy as putting the in-the-ear portion 27 into the wearer's external ear canal 904 and the bowl-shaped area of tragus-concha 905, 906, 907, and removing these auricular ECG electrodes 12, 13, will be as easy as removing the in-the-ear portion from the wearer's external ear 902. The first auricular ECG electrode 12 and the second auricular ECG electrode 13 may be configured as wireless ECG electrodes and the ECG recording module 20 may comprise a wireless ECG amplifier 21. These wireless ECG electrodes and wireless ECG amplifier may allow the first wireless auricular ECG electrode 12 and the second wireless auricular ECG electrode 13 to be housed in the in-the-ear portion 27 of the auricular housing structure 11, while the ECG recording module 20 and the processing unit 50, 401 may be housed remotely in one of the following: a wearable watch-type structure 400A and a portable smart-phone-type structure 400B. The single-ear auricular ECG system 100A may further comprise a network interface 53, 406, and at least one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on the client device 400 of the wearer 900. The network interface 53, 406 may be configured to generate a visible notification to the display screen 404C on the client device 400 of the wearer 900 describing the wearer's 900 electrocardiographic profile. The network interface 53, 406 may be configured to generate an audible notification to the speaker 15, 404A, when the processing unit 50, 401, detects presence of serious cardiac arrhythmia or impending serious cardiac arrhythmia and the network interface 53, 406, is further configured to generate a tactile notification to the vibrator 17, 404B, when the processing unit detects presence of serious cardiac arrhythmia or impending serious cardiac arrhythmia.

In some embodiments, a dual-ear auricular ECG system 100B may comprise two auricular housing structures 11 with one auricular housing structure 11 being coupled to a first ear 902 and another auricular housing structure 11 being coupled to a second ear 902. The dual-ear auricular ECG system 100B may comprise an ECG recording module 20. The ECG recording module 20 may comprise at least two wired or wireless ECG electrodes 12, 88, including a first auricular ECG electrode 12 and a contralateral auricular ECG electrode 88. The first auricular ECG electrode 12 may be housed in a first auricular housing structure 11 to be coupled to the wearer's first ear 902 or a peri-auricular area 903 around the wearer's first ear 902. The first auricular housing structure 11 may include a first in-the-ear portion 27 and the first in-the-ear portion 27 is configured to be put into the external ear canal 904 of the wearer's first ear 902 and the bowl-shaped area of tragus-concha 905, 906, 907, of the wearer's first ear 902 when in use. The first auricular ECG electrode 12 may be configured to be partially embedded in a surface 27A of the first in-the-ear portion 27 with slight protrusion at the surface 27A of the first in-the-ear portion 27. The first in-the-ear portion 27 may be configured to comprise a flexible elastic and adaptable material (such as soft foam or soft silicone-type material) and the flexible elastic and adaptable material may be configured to have appropriate flexibility, elasticity and adaptability such that the first in-the-ear portion 27 will naturally adapt to the contour of the wearer's first external ear canal 904 and the contour of the bowl-shaped area of tragus-concha 905, 906, 907, of the wearer's first ear 902 and snugly fill the interior of the wearer's first external ear canal 904 and bowl-shaped area of tragus-concha 905, 906, 907, when the first in-the-ear portion 27 is put in place and such that the first auricular ECG electrode 12 is naturally and snugly in contact with the skin of the first external ear canal 904 and skin of bowl-shaped area of tragus-concha 905, 906, 907, of the wearer's first ear 902 when the first in-the-ear portion 27 is put in place. Similarly, the contralateral auricular ECG electrode 88 may be housed in a second auricular housing structure 11 to be coupled to the wearer's second ear 902 or the peri-auricular area 903 around the wearer's second ear. The second auricular housing structure 11 may comprise a second in-the-ear portion 27 and the second in-the-ear portion 27 is configured to be put into an external ear canal 904 and a bowl-shaped area of tragus-concha 905, 906, 907, of the wearer's second ear 902 when in use. The contralateral auricular ECG electrode 88 may be configured to be partially embedded in a surface 27A of the second in-the-ear portion 27 with slight protrusion at the surface 27A of the second in-the-ear portion 27. The second in-the-ear portion 27 may be configured to comprise a flexible elastic and adaptable material and the flexible elastic and adaptable material may be configured to have appropriate flexibility, elasticity and adaptability such that the second in-the-ear portion 27 will naturally adapt to the contour of the second external ear canal 904 and the contour of the bowl-shaped area of tragus-concha 905, 906, 907, of the wearer's second ear 902 and snugly fill the interior of the second external ear canal 904 and interior of the bowl-shaped area of tragus-concha 905, 906, 907, of the wearer's second ear 902 when the second in-the-ear portion 27 is put in place and such that the contralateral auricular ECG electrode 88 is naturally and snugly in contact with the skin of the second external ear canal 904 and skin of bowl-shaped area of tragus-concha 905, 906, 907, of the wearer's second ear 902 when the second in-the-ear portion 27 is put in place. These setups will make the first auricular ECG electrode 12 and the contralateral auricular ECG electrode 88 easily self-installable and easily self-removable, similar to the aforementioned descriptions. The first auricular ECG electrode 12 and the contralateral auricular ECG electrode 88 may be configured as wireless ECG electrodes and the ECG recording module 20 may be configured to comprise a wireless ECG amplifier 21. The first wireless auricular ECG electrode 12 may be housed in the first in-the-ear portion 2 of the auricular housing structure 11, and the wireless contralateral auricular ECG electrode 88 may be housed in the second in-the-ear portion 27 of the second auricular housing structure 11, while the ECG recording module 20 and the processing unit 50, 401, may be housed in one of the following: a wearable watch-type structure 400A and a portable smart-phone-type structure 400B. The dual-ear auricular ECG system 100B further comprises a network interface 53, 406, and at least one of the following: a speaker 15, 404A, a vibrator 17, 404B, and a display screen 404C on a client device 400 of the wearer 900. The network interface 53, 406, may be in electronic communication with the processing unit 50, 401, the speaker 15, 404A, the vibrator 17, 404B, and the display screen 404C on the client device 400 of the wearer 900. The network interface 53, 406, is configured to generate a visible notification to the display screen 404C on the client device 400 of the wearer 900 describing the wearer's 900 electrocardiographic profile. The network interface 53, 406, is further configured to generate an audible notification to the speaker 15, 404A when the processing unit detects presence of a serious cardiac arrhythmia or presence of an impending serious cardiac arrhythmia. Similarly, the network interface 53, 406, is configured to generate a tactile notification to the vibrator 17, 404B when the processing unit detects presence of a serious cardiac arrhythmia or presence of an impending serious cardiac arrhythmia.

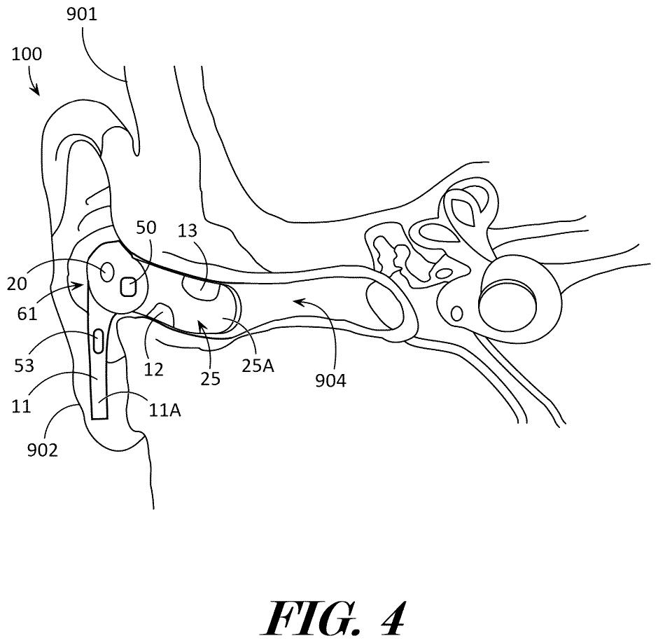

In some embodiments, a single-ear auricular ECG system 100A may comprise an auricular housing structure 11, and the auricular housing structure 11 may be configured as an earbud style structure 11A. The single-ear auricular ECG system 100A comprises at least two auricular ECG electrodes 12, 13 (for example a first auricular ECG electrode 12 and a second auricular ECG electrode 13). The auricular ECG electrodes 12, 13, may be placed on a surface 25A of a first horizontal portion 25 of a first earbud style structure 11A and the first horizontal portion 25 is configured to be placed into an external ear canal 904 and an area surrounding the opening of the external ear canal 904 (i.e. a bowl-shaped area of tragus-concha 905, 906, 907) of the wearer's first ear 902 when in use. Preferably, the first horizontal portion 25 of the first earbud style structure 11A may be made with or may comprise an elastic flexible and adaptable material (such as soft foam or very soft silicone type material), in which the material for the first horizontal portion 25 of the first earbud style structure 11A is configured to have appropriate elasticity, flexibility and adaptability such that when the first horizontal portion 25 of the first earbud-style structure 11A is put into the external ear canal 904 and the bowl-shaped area of tragus-concha 905, 906, 907, of the wearer's first ear 902 the first horizontal portion 25 of the first earbud style structure 11A will naturally adapt to the contour of wearer's external ear canal 904 and contour of the bowl-shaped area of tragus-concha 905, 906, 907, of the wearer's first ear and will snugly fill the interior of the wearer's external ear canal 904 and interior of the bowl-shaped area of tragus-concha 905, 906, 907 of the wearer's first ear. The auricular ECG electrodes 12, 13, are configured to be partially embedded in a surface 25A of the first horizontal portion 25 with slight protrusion at the surface 25A of the first horizontal portion 25 such that when the first horizontal portion 25 is put into an external ear canal 904 or bowl-shaped area of tragus-concha 905, 906, 907, of the wearer's first ear 902, the auricular ECG electrodes 12, 13, will be naturally and snugly contacting the skin of the external ear canal 904 and skin of the bowl-shaped area of tragus-concha 905, 906, 907, of the wearer's first ear. This set-up and the elasticity, flexibility and adaptability of the material will allow all of these auricular electrodes 12, 13, to be naturally in close contact with the skin of the wearer's external ear canal 904 and skin of bowl-shaped area of tragus-concha 905, 906, 907. (FIG. 4). For the wearer 900, attaching and removing these auricular electrodes 12, 13, will be as easy as placing and removing the first earbud-style structure 11A from the wearer's external ear 902. There will be no need for a certified technologist to apply the electrodes. Applying adhesive material to secure these auricular electrodes 12, 13, will also be unnecessary. This is feasible due to the unique anatomical features of a human external ear canal 904 and the area surrounding the opening of the human external ear canal. These features of easy self-installation and easy self-removal will create huge convenience for the wearer 900.

Similarly, in some embodiments, a dual-ear auricular ECG system 100B may comprise two auricular housing structures 11 with one auricular housing structure 11 being coupled to a first ear 902 and another auricular housing structure 11 being coupled to a second ear 902. The auricular housing structures 11 may be selected from one of the followings: an earbud-style structure 11A, a tubular-shaped structure 11B and a behind-the-ear-hearing-aid-style structure 11C. In some embodiments, a dual-ear auricular ECG system 100B may comprise two earbud-style housing structures 11A with one earbud-style structure 11A being coupled to a first ear 902 and another earbud-style structure 11A being coupled to a second ear 902. The dual-ear auricular ECG system 100B comprises at least two auricular ECG electrodes 12, 88 (for example a first auricular ECG electrode 12 and a contralateral auricular ECG electrode 88). The first auricular ECG electrode 12 may be placed on a surface 25A of a horizontal portion 25 of the earbud style structure 11A and the horizontal portion 25 is configured to be put into an external ear canal 904 and a bowl-shaped area of tragus-concha 905, 906, 907, of the wearer's first ear 902 when in use. The contralateral auricular ECG electrode 88 may be placed on a surface 25A of a horizontal portion 25 of another earbud style structure 11A and the horizontal portion 25 is configured to be placed inside an external ear canal 904 and a bowl-shaped area of tragus-concha 905, 906, 907, of the wearer's second ear 902 when in use. Preferably, the horizontal portions 25 of the two earbud style structures 11A may be made with or may comprise an elastic flexible and adaptable material (such as soft foam or very soft silicone type material) and the auricular ECG electrodes 12, 88, are partially embedded in the surfaces 25A of the horizontal portions 25 with slight protrusion at the surfaces 25A of the horizontal portions 25 such that these auricular ECG electrodes 12, 88, will naturally and snugly in close contact with the skin of the wearer's external ear canals 904 and the skin of the bowl-shaped areas of tragus-concha 905, 906, 907, when these earbud-style structures 11A are put in place. These setups will allow these auricular ECG electrodes 12, 88 to be easily self-installable and self-removable, similar to the descriptions hereinbefore.

In preferred embodiments, an auricular automatic anti-arrhythmia system 200A, may comprise a single-ear auricular ECG system 100A, a taVNS unit 30 and a processing unit 50, 401. The single-ear auricular ECG system 100A comprises an ECG recording module 20 that comprises at least two wired or wireless ECG electrodes 12, 13 (including a first auricular ECG electrode 12 and a second auricular ECG electrode 13). The taVNS unit 30 comprises a stimulating electrode 31. The first auricular ECG electrode 12, the second auricular ECG electrode 13 and the stimulating electrode 31 of the taVNS unit 30 may be configured to be housed in a first auricular housing structure 11. The first auricular housing structure 11 includes a first in-the-ear portion 27 and the first in-the-ear portion 27 is configured to be put into an external ear canal 904 and a bowl-shaped area of tragus-concha 905, 906, 907, of the wearer's first ear 902 when in use. The first auricular ECG electrode 12, the second auricular ECG electrode 13 of the single-ear auricular ECG system 100A and the stimulating electrode 31 of the taVNS unit 30 are configured to be partially embedded in a surface 27A of the first in-the-ear portion 27 with slight protrusion at the surface 27A of the first in-the-ear portion 27. The first in-the-ear portion 27 is configured to be made of or may comprise a flexible elastic and adaptable material and the flexible elastic and adaptable material is configured to have appropriate flexibility, elasticity and adaptability such that the first in-the-ear portion 27 will naturally adapt to the contour of the external ear canal 904 and contour of the bowl-shaped area of tragus-concha 905, 906, 907, of the wearer's 900 first ear 902 and snugly fill the interior of the external ear canal 904 and interior of the bowl-shaped area of tragus-concha 905, 906, 907, of the wearer's 900 first ear 902 when the first in-the-ear portion 27 is put in place and such that the first auricular ECG electrode 12, the second auricular ECG electrode 13 and the stimulating electrode 31 of the taVNS unit 30 are naturally and snugly in close contact with the skin of the external ear canal 904 and skin of bowl-shaped area of tragus-concha 905, 906, 907, of the wearer's first ear 902 when the first in-the-ear portion 904 is put in place. These features will allow easy self-installation and self-removal of the auricular ECG electrodes 12, 13 and the stimulating electrode 31 of the taVNS unit 30. These features will also enable the stimulating electrode 31 of the taVNS unit 30 to be naturally in contact with vagus innervated auricular skin since the external ear canal 904, tragus 905, and concha 906, 907, all received rich innervation from the auricular branch of vagus nerve, as described hereinbefore. The first auricular ECG electrode 12 and the second auricular ECG electrode 13 of the single-ear auricular ECG system 100A may be configured as wireless ECG electrodes and the ECG recording module 20 may be configured to comprise a wireless ECG amplifier 21 to allow the first wireless auricular ECG electrode 12 and the second wireless auricular ECG electrode 13 to be housed in the first in-the-ear portion 27 of the first auricular housing structure 11 while the ECG recording module 20 and the processing unit 50, 401 may be housed in one of the following: a wearable watch-type structure and a portable smart-phone-type structure. The auricular automatic anti-arrhythmia system 200A may further comprise a network interface 53, 406, and at least one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. The network interface 53, 406 is configured to generate a visible notification to the display screen 404C on the client device 400 of the wearer 900 describing the wearer's electrocardiographic profile. The network interface 53, 406, is further configured to generate an audible notification to the speaker 15, 404A, when the processing unit 50, 401, detects presence of a serious cardiac arrhythmia or presence of an impending serious cardiac arrhythmia, and the network interface 53, 406, is also configured to generate a tactile notification to the vibrator 17, 404B, when the processing unit 50, 401, detects presence of a serious cardiac arrhythmia or presence of an impending serious cardiac arrhythmia.

In some embodiments, an auricular automatic anti-arrhythmia system 200A, may comprise a dual-ear auricular ECG system 100B, a taVNS unit 30 and a processing unit 50, 401. The dual-ear auricular ECG system 100B comprises an ECG recording module 20 that comprises at least two auricular ECG electrodes 12, 88, (including a first auricular ECG electrode 12 and a contralateral auricular ECG electrode 88). The taVNS unit 30 comprises a stimulating electrode 31. The first auricular ECG electrode 12 and the stimulating electrode 31 of the taVNS unit 30 may be configured to be housed in a first auricular housing structure 11 to be coupled to the wearer's first ear 902 or the peri-auricular area 903 around the wearer's first ear 902. The first auricular housing structure 11 comprises a first in-the-ear portion 27 and the first in-the-ear portion 27 is configured to be placed into an external ear canal 904 and a bowl-shaped area of tragus-concha 905, 906, 907, of the wearer's first ear 902 when in use. The first auricular ECG electrode 12 of the dual-ear auricular ECG system 100B and the stimulating electrode 31 of the taVNS unit 30 may be configured to be partially embedded in a surface 27A of the first in-the-ear portion 27 with slight protrusion at the surface 27A of the first in-the-ear portion 27. The first in-the-ear portion 27 may be configured to comprise or be made with a flexible elastic and adaptable material and the flexible elastic and adaptable material is configured to have appropriate flexibility, elasticity and adaptability such that the first in-the-ear portion 27 will naturally adapt to the contour of the external ear canal 904 and contour of the bowl-shaped area of tragus-concha 905, 906, 907, of the wearer's first ear 902 and snugly fill the interior of the external ear canal 904 and interior of the bowl-shaped area of tragus-concha 905, 906, 907, of the wearer's first ear 902 when the first in-the-ear portion 27 is put in place and such that the first auricular ECG electrode 12 and the stimulating electrode 31 of the taVNS unit 30 are naturally and snugly in close contact with the skin of the external ear canal 904 and skin of bowl-shaped area of tragus-concha 905, 906, 907, of the wearer's first ear 902 when the first in-the-ear portion 27 is put into the wearer's first ear 902. The contralateral auricular ECG electrode 88 of the dual-ear auricular ECG system 100B may be configured to be housed in a second auricular housing structure 11 to be coupled to the wearer's second ear 902 or the peri-auricular area 903 around the wearer's second ear 902. The second auricular housing structure 11 comprises a second in-the-ear portion 27 and the second in-the-ear portion 27 is configured to be put into an external ear canal 904 and a bowl-shaped area of tragus-concha 905, 906, 907, of the wearer's second ear 902 when in use. The contralateral auricular ECG electrode 88 is configured to be partially embedded in a surface 27A of the second in-the-ear portion 27 with slight protrusion at the surface 27A of the second in-the-ear portion 27. The second in-the-ear portion 27 may be configured to comprise or be made with a flexible elastic and adaptable material and the flexible elastic and adaptable material is configured to have appropriate flexibility, elasticity and adaptability such that the second in-the-ear portion 27 will naturally adapt to the contour of the external ear canal 904 and contour of the bowl-shaped area of tragus-concha 905, 906, 907, of the wearer's second ear 902 and snugly fill the interior of the external ear canal 904 and interior of the bowl-shaped area of tragus-concha 905, 906, 907, of the wearer's second ear 902 when the second in-the-ear portion 27 is put into the wearer's second ear 902 and such that the contralateral auricular ECG electrode 88 will be naturally and snugly in close contact with the skin of the external ear canal 904 and skin of the bowl-shaped area of tragus-concha 905, 906, 907, of the wearer's second ear 902 when the second in-the-ear portion 27 is put into the wearer's second ear 902. These setups will allow the first auricular ECG electrode 12, the contralateral auricular ECG electrode 88 and the stimulating electrode 31 of the taVNS unit 30 to be easily self-installable and self-removable and the stimulating electrode 31 will be naturally contacting the skin of vagus innervated auricular skin when put in use. The first auricular ECG electrode 12 and the contralateral auricular ECG electrode 88 of the dual-ear auricular ECG system 100B are configured as wireless ECG electrodes and the ECG recording module 20 is configured to comprise a wireless ECG amplifier 21 to allow the first wireless auricular ECG electrode 12 to be housed in the first auricular housing structure 11 and the wireless contralateral auricular ECG electrode 88 to be housed in the second auricular housing structure 11 while the ECG recording module 20 to be housed remotely in a client device 400 (such as a wearable watch-type device 400A or a portable smart phone type device 400B). (Please note that the stimulating electrode 31 of the taVNS unit 30 may be housed together with the contralateral auricular ECG electrode 88 in the second auricular housing structure 11.) The auricular automatic anti-arrhythmia system 200A may further comprise a network interface 53, 406, and at least one of the following: a speaker 15, 404A, a vibrator 17, 404B, and a display screen 404C on a client device 400 of the wearer 900. The network interface 53, 406, may be in electronic communication with the processing unit 50, 401, the speaker 15, 404A, the vibrator 17, 404B, and the display screen 404C on the client device 400 of the wearer 900. The network interface 53, 406, is configured to generate a visible notification to the display screen 404C on the client device 400 of the wearer 900 describing the wearer's 900 electrocardiographic profile. The network interface 53, 406 is also configured to generate an audible notification to the speaker 15, 404A when the processing unit 50, 401, detects presence of serious cardiac arrhythmia or presence of impending serious cardiac arrhythmia. The network interface 53, 406, may be further configured to generate a tactile notification to the vibrator 17, 404B when the processing unit 50, 401, detects presence of serious cardiac arrhythmia or presence of impending serious cardiac arrhythmia.

The auricular housing structures 11 may be selected from one of the following: earbud-style structure(s) 11A, tubular-shaped structure(s) 11B and behind-the-ear-hearing-aid-style structure(s) 11C. These auricular housing structures 11 are inherently stable and suitable for long-term attachment. They can be easily removed temporarily to charge the power sources 16 (batteries). They can also be easily put back into place. Since cardiac arrhythmia is usually a long-lasting or permanent problem, a long-term (months or years) way to securely attach an auricular ECG monitoring system 100A, 100B, and a taVNS unit 30 to a wearer 900 is greatly needed. No surgery would be needed. Nowadays, earphones, air-pods and earbuds have become quite popular. They are nice-looking and well accepted by most people. (FIGS. 4, 5, 6, 7, 8, 9). These housing structures enable convenient long-term monitoring and automatic therapeutic intervention of the cardiac arrhythmias.

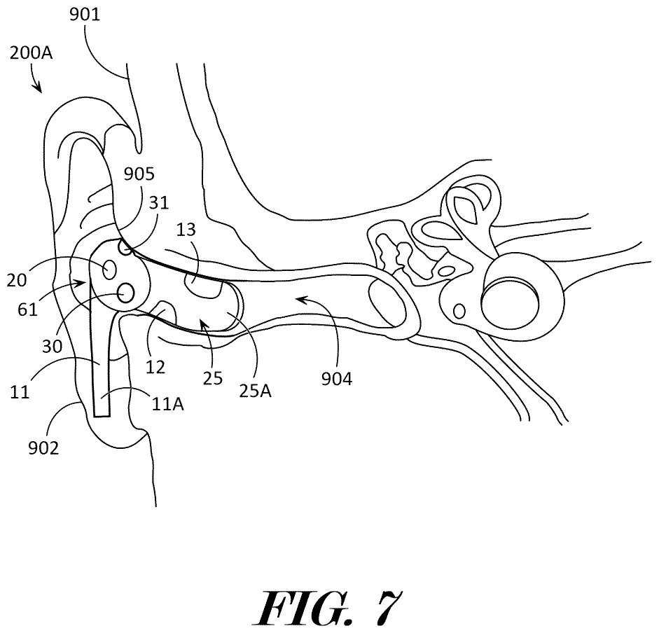

In some embodiments, an auricular automatic anti-arrhythmia system 200A, comprising a single-ear auricular ECG system 100A and a taVNS unit 30, may comprise an auricular housing structure 11 selected from one of the followings: an earbud-style structure 11A, a tubular-shaped structure 11B and a behind-the-ear-hearing-aid-style structure 11C. The auricular ECG electrodes 12, 13, may be configured to be housed in a first in-the-ear portion 27 of a first auricular housing structure 11. The ECG recording module 20 may be configured to be housed in the first auricular housing structure 11 or be housed in a client device 400 such as a wearable watch-type device 400A or a portable smart phone type device 400B. The taVNS unit 30 may be housed together with the auricular ECG electrodes 12, 13, in the first auricular housing structure 11. Alternatively, the taVNS unit may be housed in a taVNS housing 40 that may be coupled to or integrally formed with the first auricular housing structure 11. In some embodiments, the auricular ECG electrodes 12, 13 and the taVNS unit 30 (including the stimulating electrode 31 of the taVNS unit 30) may be housed together in a first in-the-ear portion 27 of a first auricular housing structure 11 that may be configured as a first earbud style structure 11A. The auricular ECG electrodes 12, 13, and the taVNS stimulating electrode 31 may be placed on a surface 25A of a first horizontal portion 25 of the first earbud style structure 11A. Preferably, the first horizontal portion 25 of the first earbud style structure 11A may be made with or may comprise an elastic flexible and adaptable material (such as soft foam or very soft silicone type material), in which the material for the horizontal portion 25 of the first earbud style structure 11A is configured to have appropriate elasticity, flexibility and adaptability such that when the first horizontal portion 25 of the first earbud-style structure 11A is put into an external ear canal 904 and a bowl-shaped area of tragus-concha 905, 906, 907, of the wearer's first ear 902 the first horizontal portion 25 of the first earbud style structure 11A will naturally adapt to the contour of the external ear canal 904 and contour of the bowl-shaped area of tragus-concha 905, 906, 907, of the wearer's first ear 902 and will snugly fill the interior of the external ear canal 904 and interior of the bowl-shaped area of tragus-concha 905, 906, 907, of the wearer's first ear 902. The auricular ECG electrodes 12, 13, and the stimulating electrode 31 of the taVNS unit 30 are partially embedded in a surface 25A of the first horizontal portion 25 with slight protrusion at the surface 25A of the first horizontal portion 25 such that when the first horizontal portion 25 is put into an external ear canal 904 or a bowl-shaped area of tragus-concha 905, 906, 907, of a wearer's first ear 902, the auricular ECG electrodes 12, 13 and the stimulating electrode 31 of the taVNS unit 30 will be naturally and snugly in closed contact with the skin of the external ear canal 904 and skin of bowl-shaped area of tragus-concha 905, 906, 907, of the wearer's first ear 902. This set-up and the elasticity, flexibility and adaptability of the material will allow all of these electrodes 12, 13, 31, to be naturally in close contact with the skin of the wearer's external ear canal 904 and skin of bowl-shaped area of tragus-concha 905, 906, 907. (FIGS. 4, 7). At the same time, the taVNS stimulating electrode 31 will also be naturally in close contact with vagus innervated auricular skin since external ear canal 904, tragus 905, concha 906, 907, all have rich innervation from the auricular branch of vagus nerve. For the wearer 900, attaching and removing these electrodes 12, 13, 31, will be as easy as placing and removing the first earbud-style structure 11A from the wearer's first ear 902. There will be no need for a certified technologist to apply the electrodes. Applying adhesive material to secure these electrodes 12, 13, 31, will also be unnecessary. This is feasible due to the unique anatomical features of the human external ear canal 904, as illustrated in FIGS. 4, and 7. These features of easy self-installation and easy self-removal will create huge convenience for the wearer.

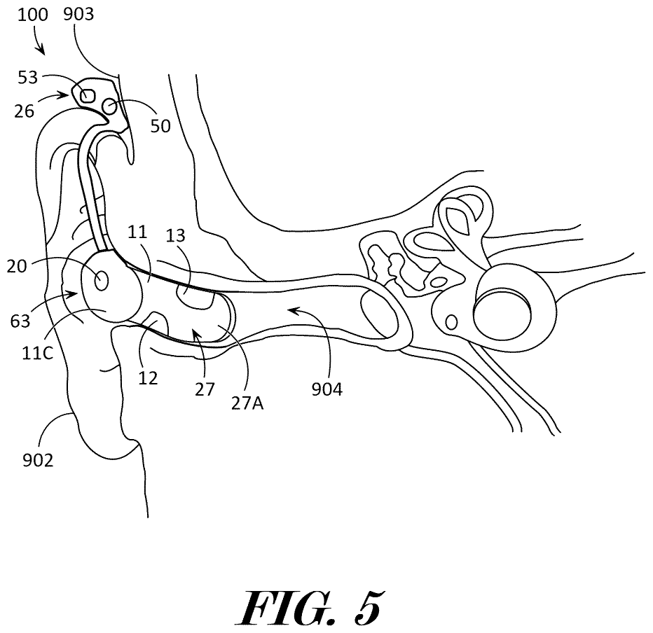

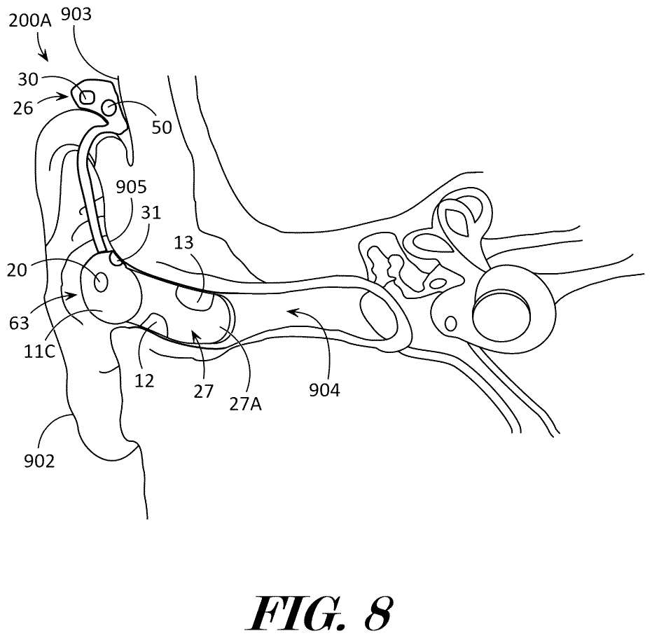

These advantages can be similarly achieved when an auricular housing structure 11 is configured as a behind-the-ear-hearing-aid-style structure 11C for a single-ear auricular ECG monitoring system 100A and/or an auricular automatic anti-arrhythmia system 200A that comprises a single-ear auricular ECG system 100A and a taVNS unit 30. (Example of a behind-the-ear-hearing-aid style structure 11C: Example behind-the-ear structures 11C include behind-the-ear (BTE) receiver-in-the-ear (RITE), CROS/BICROS (CROS stands for “Contralateral Routing of Signals” and BiCROS stands for “Bilateral Contralateral Routing of Signals”), and the like.) The behind-the-ear-hearing-aid-style structure 11C includes an in-the-ear portion 27 and a behind-the-ear portion 26. (As used herein, an in-the-ear portion 27 of a behind-the-ear-hearing-aid-style structure 11C is essentially the same as an in-the-ear portion 27 of an auricular housing structure 11.) In some embodiments, an auricular automatic anti-arrhythmia system 200A may comprise a first behind-the-ear-hearing-aid-style structure 11C for housing and the auricular ECG electrodes 12, 13 and the stimulating electrode 31 of the taVNS unit 30 are configured to be placed on a surface 27A of a first in-the-ear portion 27 of the first behind-the-ear-hearing-aid-style structure 11C. Preferably, the first in-the-ear portion 27 of the first behind-the-ear-hearing-aid-style structure 11C may be made with or may comprise an elastic flexible and adaptable material (such as soft foam or very soft silicone type material), in which the material for the first in-the-ear portion 27 is configured to have appropriate elasticity, flexibility and adaptability such that when the first in-the-ear portion 27 is put into an external ear canal 904 and a bowl-shaped area of tragus-concha 905, 906, 907, of the wearer's first ear 902 the first in-the-ear portion 27 will naturally adapt to the contour of external ear canal 904 and contour of the bowl-shaped area of tragus-concha 905, 906, 907, of the wearer's first ear and will snugly fill the interior of the external ear canal 904 and interior of bowl-shaped area of tragus-concha 905, 906, 907, of the wearer's first ear 902. The auricular ECG electrodes 12, 13, and the stimulating electrode 31 of the taVNS unit 30 are partially embedded in a surface 27A of the first in-the-ear portion 27 with slight protrusion at the surface 27A of the first in-the-ear portion 27 such that when the first in-the-ear portion 27 is put in place, the auricular ECG electrodes 12, 13 and the stimulating electrode 31 of the taVNS unit 30 will be naturally and snugly in close contact with the skin of the external ear canal 904 and skin of bowl-shaped area of tragus-concha 905, 906, 907, of the wearer's first ear 902. These set-ups and the elasticity, flexibility and adaptability of the material will allow all of these electrodes 12, 13, 31, to be naturally in close contact with the skin of the external ear canal 904 and skin of bowl-shaped area of tragus-concha 905, 906, 907, of the wearer's first ear. (FIGS. 5, 8). At the same time, the taVNS stimulating electrode 31 will also be naturally in close contact with vagus innervated auricular skin since the skin of external ear canal 904, tragus 905, concha 906, 907 all received rich innervation from the auricular branch of vagus nerve. For the wearer 900, attaching and removing these electrodes 12, 13, 31, will be as easy as inserting and removing the first behind-the-ear-hearing-aid-style structure 11C from the wearer's first ear 902. There will be no need for a certified technologist to apply the electrodes. Applying adhesive material to secure these electrodes 12, 13, 31, will also be unnecessary. This is feasible due to the unique anatomical features of the human external ear canal 904, as illustrated in FIGS. 5, and 8. These features of easy self-installation and easy self-removal will create huge convenience for the wearer. Likewise, the above advantages can be achieved when a behind-the-ear-hearing-aid-style structure 11C is utilized for housing for a dual-ear auricular ECG system 100B and/or an auricular automatic anti-arrhythmia system 200A that comprises a dual-ear auricular ECG system 100B, similar to aforementioned descriptions.

The aforementioned advantages can also be similarly achieved when an auricular housing structure 11 is configured as a tubular-shaped structure 11B for a single-ear auricular ECG system 100A and/or an auricular automatic anti-arrhythmia system 200A that comprises a single-ear ECG system 100A and a taVNS unit 30. The auricular ECG electrodes 12, 13, and the stimulating electrode 31 of the taVNS unit 30 are configured to be located at a surface 29 of the tubular-shaped structure 11B. Preferably, the tubular-shaped structure 11B may be made with or may comprise elastic flexible and adaptable material (such as soft foam or very soft silicone), in which the material for the tubular-shaped structure 11B is configured to have appropriate elasticity flexibility and adaptability such that when the tubular-shaped structure 11B is inserted into a wearer's external ear canal 904, the tubular-shaped structure 11B will naturally adapt to the contour of the wearer's external ear canal 904 and will snugly fill the interior of the wearer's external ear canal 904. The auricular ECG electrodes 12, 13, and the stimulating electrode 31 of the taVNS unit 30 are partially embedded in a surface 29 of the tubular-shaped structure with slight protrusion at the surface 29 of the tubular-shaped structure 11B such that when the tubular-shaped structure 11B is inserted into an external ear canal 904 of a wearer 900, the ECG electrodes 12, 13 and the stimulating electrode 31 of the taVNS unit 30 will be naturally and snugly contacting the skin of the wearer's external ear canal 904. This set-up and the elasticity flexibility and adaptability of the material will enable all of these electrodes 12, 13, 31 to be naturally in close contact with the skin of the wearer's external ear canal 904. The taVNS stimulating electrode 31 will also be naturally in close contact with the wearer's vagus innervated auricular skin since the skin of the external ear canal 904 is part of the vagus innervated auricular skin. These features of easy self-installation and easy self-removal will create huge convenience for the wearer. Likewise, the above advantages can be achieved when a tubular-shaped structure 11B is utilized for housing for a dual-ear auricular ECG system 100B and/or an auricular automatic anti-arrhythmia system 200A that comprises a dual-ear auricular ECG system 100B, similar to aforementioned descriptions.

In some embodiments, an auricular automatic anti-arrhythmia system 200A may comprise a dual-ear auricular ECG system 100B and two taVNS unit 30 (for example a first taVNS unit 30 and a second taVNS unit 30). This system is called bilateral auricular anti-arrhythmia system 200A hereinafter. (Bilateral taVNS nerve stimulation has been reported as more effective than unilateral taVNS nerve stimulation.) In some embodiments, a bilateral auricular anti-arrhythmia system 200A may comprise two auricular housing structures 11 with one auricular housing structure 11 being coupled to a first ear 902 of a wearer 900 and another auricular housing structure 11 being coupled to a second ear 902 of the wearer 900, as shown on FIG. 21. Each auricular housing structure 11 includes an in-the-ear portion 27 that is configured to be put into an external ear canal 904 and a bowl-shaped area of tragus-concha 905, 906, 907, when in use. The auricular housing structures 11 may be selected from one of the followings: an earbud-style structure 11A, a tubular-shaped structure 11B and a behind-the-ear-hearing-aid-style structure 11C. In some embodiments, a bilateral auricular anti-arrhythmia system 200A may comprise two earbud-style structures 11A for housing with one earbud-style structure 11A being coupled to a first ear 902 of the wearer 900 and another earbud-style structure 11A being coupled to a second ear 902 of the wearer 900. The dual-ear auricular ECG system 100B of the bilateral auricular anti-arrhythmia system 200A comprises at least two auricular ECG electrodes 12, 88 (for example a first auricular ECG electrode 12 and a contralateral auricular ECG electrode 88). The first auricular ECG electrode 12 and a stimulating electrode 31 of the first taVNS unit may be placed on a surface 25A of a horizontal portion 25 of the earbud style structure 11A to be coupled to the first ear 902 of the wearer. The contralateral auricular ECG electrode 88 and a stimulating electrode 31 of the second taVNS unit 30 may be placed on a surface 25A of a horizontal portion 25 of another earbud style structure 11A to be coupled to the second ear 902 of the wearer 900. Preferably, the horizontal portion 25 of the first earbud style structure 11A may be made with or may comprise an elastic flexible and adaptable material (such as soft foam or very soft silicone type material) and the first auricular ECG electrode 12 and the stimulating electrode 31 of the first taVNS unit 30 are partially embedded in the surface 25A of the horizontal portion 25 with slight protrusion at the surface 25A of the horizontal portion 25 such that the first auricular ECG electrode 12 and the stimulating electrode 31 of the first taVNS unit 30 can be easily self-installed and self-removed from the first ear 902 of the wearer 900, similar to the descriptions hereinbefore. Likewise, the horizontal portion 25 of the second earbud style structure 11A may be made with or may comprise an elastic flexible and adaptable material (such as soft foam or very soft silicone type material) and the contralateral auricular ECG electrode 88 and the stimulating electrode 31 of the second taVNS unit 30 are partially embedded in the surface 25A of the horizontal portion 25 of the second earbud style structure 11A with slight protrusion at the surface 25A of the horizontal portion 25 of the second earbud-style structure 11A such that the contralateral auricular ECG electrode 88 and the stimulating electrode 31 of the second taVNS unit 30 can be easily self-installed and self-removed from the second ear 902 of the wearer 900, similar to the descriptions hereinbefore. These features and advantages can be similarly achieved when bilateral tubular-shaped structures 11B are used for housing for a bilateral auricular anti-arrhythmia system 200A or when bilateral behind-the-ear-hearing-aid-style structures 11C are used for housing for a bilateral auricular anti-arrhythmia system 200A, essentially similar to the descriptions hereinbefore. Similar features and advantages can also be achieved when using mixed housing structures 11 (for example one ear with earbud-style housing 11A and the other ear with tubular-shaped structure housing 11B or behind-the-ear-hearing-aid-style housing 11C or other mixtures).

It should be noted that the auricular ECG monitoring systems 100 (including the single-ear auricular ECG system 100A and the dual-ear auricular ECG system 100B) contain self-installable and self-removable auricular ECG electrodes 12, 13, 88, and these systems are wearable, due to the features and set-ups of the electrode-housing structure (in-the-ear portion 27 of the auricular housing structure 11). Likewise, the auricular automatic anti-arrhythmia system 200A with a single-ear ECG system 100A and a taVNS unit 30 also contains self-installable auricular ECG electrodes 12, 13, and self-installable ta VNS stimulating electrode 31 and the systems 100A and 200A are fully self-installable and self-removable. Similarly, the auricular automatic anti-arrhythmia system 200A with a dual-ear ECG system 100B and a taVNS unit 30 also contains self-installable auricular ECG electrodes 12, 88, and self-installable taVNS stimulating electrode 31 and the systems 100B and 200A is fully self-installable and self-removable. There is no need for a technologist to perform installation or removal of electrodes. These are due to the set-ups and features of the electrode-housing structures 11, 40. Users of these wearable systems can ambulate freely.

In some embodiments, a separate client device 400 may be used for housing of one or more of the components of an auricular automatic anti-arrhythmia system 200A. For example, an ECG recording module 20 and the processing unit 50, 401, may be housed remotely in a client device 400, such as a wearable smart watch-type structure 400A or a portable smart phone type device 400B. The auricular ECG electrodes 12, 13, 88, and the taVNS stimulating electrode 31 may be housed in one or two auricular housing structure(s) 11 (such as an earbud-type housing 11A, an in-the-ear portion 27 of a behind-the-ear-hearing-aid-style housing 11C or a tubular-shaped structure 11B.) Wireless ECG electrodes may be used for all of the ECG electrodes 12, 13, 88. Wireless dry electrodes 12, 13, 88, are known in the art, such as that described by Ryan Kaveh et al. in Nature Communications on Aug. 2, 2024. Wireless dry ECG electrodes are available from various companies, such as iWork System, BIOPAC and PASCO Scientific that offer various wireless ECG monitoring systems, including wireless ECG electrodes. There are well-known wired or wireless ECG amplifiers available, such as ECG Electrocardiogram Smart Amplifier (Part #ECG100D), or BioNomadix 2Ch Wireless ECG Amplifier (Part #BN-ECG2). Both are also available from BIOPAC Systems, Inc. (Goleta, California). By using wireless ECG electrodes and wireless ECG amplifier, the ECG recording module 20, together with the processing unit 401, may be housed remotely in a client device 400 (such as a wearable watch-type client device 400A or a portable smart phone type client device 400B) and communicate wirelessly with all of the auricular ECG electrodes 12, 13, 88. In some embodiments, an auricular automatic anti-arrhythmia system 200A may comprise a client device 400 having a processing unit 401 that the taVNS unit 30 and/or processing unit 50 of an auricular ECG monitoring system 100 may be in electronic communication 19 with. An auricular automatic anti-arrhythmia system 200A may comprise an auricular ECG monitoring system 100 and a taVNS unit 30 that may both be in communication with a processing unit 50, 401, through Bluetooth, Wi-Fi, a wired connection, or other electronic communication 19 connecting means via a communication interface 42. The recorded ECG data recorded by the auricular ECG monitoring system 100 are preferably transmitted to the one or more processing units 50, 401, of the auricular automatic anti-arrhythmia system 200A. With the help of automatic ECG analysis algorithms, the processing unit 50, 401, is configured to analyze the ECG data to detect any serious cardiac arrhythmia, or any impending serious cardiac arrhythmia, including AFib, AFL, SVT, VT, VF, etc., (such as via existing ECG analysis algorithms). Upon detection of serious cardiac arrhythmia, the processing unit 50, 401, may be further configured to automatically send signals (arrhythmia-warning signals) to the taVNS unit 30 to actuate the taVNS unit 30 to immediately start sending pre-determined electric stimuli to the auricular branch of vagus nerve via the vagus innervated auricular skin that the stimulating electrode 31 is in contact with (stimulation parameters as in Table 1). When the processing unit 50, 401, detects presence of impending serious cardiac arrhythmia, the processing unit 50, 401, is configured to automatically send signals (impending-arrhythmia-warning signals) to the taVNS unit 30 to actuate the ta VNS unit 30 to immediately start sending pre-determined electric stimulation to the auricular branch of vagus nerve via the vagus innervated auricular skin to which the stimulating electrode 31 is in contact with (stimulation parameters as in Table 2). When the processing unit 50, 401, detects cessation of serious cardiac arrhythmia or cessation of impending serious cardiac arrhythmia, the processing unit 50, 401, may also be configured to send or electronically communicate signals (arrhythmia-cessation signals or impending-arrhythmia-cessation signals) to the taVNS unit 30 to modify or stop the electric stimuli to the vagus nerve via the stimulating electrode 31. This enables the auricular automatic anti-arrhythmia system 200A to automatically initiate therapeutic intervention instantly. This can avoid the inevitable delay in traditional set-up where the ECG data being sent to a monitoring center and the monitoring center notifying healthcare provider 950 via their client device 400 to arrange for therapeutic intervention.

In some embodiments, an auricular ECG monitoring system 100 (including 100A and 100B) and/or an auricular automatic anti-arrhythmia system 200A may comprise a network interface 53, 406. The network interface 53, 406 is in electronic communication with the processing unit 50, 401. The network interface 53, 406 may also be in electronic communication with at least one of the following: a speaker 15, 404A, a vibrator 17, 404B, and a display screen 89, 404C on a client device 400. When the processing unit 50, 401 detects presence of serious cardiac arrhythmia or presence of impending serious cardiac arrhythmia, the network interface 53, 406, is configured to send signals to at least one of: the speaker 15, 404A, the vibrator 17, 404B and the display screen 404C. The speaker 15, 404A may be configured to generate an audible notification to a wearer 900 when serious cardiac arrhythmia or impending serious cardiac arrhythmia is detected by the processing unit 50, 401. The vibrator 17, 404B is configured to generate a tactile notification to the wearer 900 when serious cardiac arrhythmia or impending serious cardiac arrhythmia is detected by the processing unit. The display screen 404C is configured to generate a visible display to a client device 400 of the wearer 900 or the wearer's healthcare provider 950 with visible notification of the wearer's serious cardiac arrhythmia or impending serious cardiac arrhythmia. When the processing unit 50, 401 detects cessation of the serious cardiac arrhythmia or impending serious cardiac arrhythmia, the network interface 53, 406 may be further configured to send signals to the speaker 15, 404A, the vibrator 17, 404B and the display screen 404C to stop the notification of serious cardiac arrhythmia or impending serious cardiac arrhythmia.

The auricular ECG monitoring system 100 is an excellent example of a wearable ECG monitoring system that can be used for long-term ECG monitoring purpose. There are other ECG monitoring systems 250, 350, 450, that are also wearable and suitable for monitoring purpose. Examples of wearable ECG monitoring systems may include a chest-based ECG monitoring system 250, an arm-based ECG monitoring system 350, and smart-ring ECG monitoring systems 450 (including ear-finger ECG system 450A, finger-finger ECG system 450B, ear-toe ECG system 750A and finger-toe ECG system 750B) and each of them may be a component of an automatic anti-arrhythmia system (including chest-based automatic anti-arrhythmia system 200B, an arm-based automatic anti-arrhythmia system 200C, an ear-finger automatic anti-arrhythmia system 460A, a finger-finger automatic anti-arrhythmia system 460B, an ear-toe automatic anti-arrhythmia system 760A and a finger-toe automatic anti-arrhythmia system 760B).

In some embodiments, a chest-based automatic anti-arrhythmia system 200B (the system 200B) may comprise a chest-based ECG monitoring system 250, a transcutaneous auricular vagus nerve stimulation (taVNS) unit 30 and a processing unit 50, 401. The processing unit 50, 401 is in electronic communication with the chest-based ECG monitoring system 250 and the taVNS unit 30. The taVNS unit 30 may be in electronic communication 19 with the chest-based ECG monitoring system 250. There are well-known chest-based ECG monitoring systems 250, such as a Holter monitor, a Zio Patch Monitor or an Assert-IQ ICM from Abbott, having its headquarters in Lake County, Illinois. Generally, a chest-based ECG monitoring system 250, like a Holter monitor, may comprise a device that may be placed on the chest 910 of a wearer 900 with a plurality of electrodes 251 (chest-contacting electrodes 251) attached to the wearer's chest 910, allowing it to record the electrical activity of the heart by picking up signals directly from the chest area 910; essentially, the plurality of chest-contacting electrodes 251 are positioned on the chest 910 to capture the heart's electrical signals. With the help of automatic ECG analysis algorithms, the processing unit 50, 401, is configured to analyze the ECG data to detect any serious cardiac arrhythmia or any impending serious cardiac arrhythmia, including AFib, AFL, SVT, VT, VF, etc., (such as via existing ECG analysis algorithms). Upon detection of serious cardiac arrhythmia, the processing unit 50, 401, may be further configured to automatically send or electronically communicate signals (arrhythmia-warning signals) to the taVNS unit 30 to actuate the taVNS unit 30 immediately to start sending pre-determined electric stimuli to the auricular branch of vagus nerve via the vagus-innervated auricular skin that the stimulating electrode 31 is in contact with (stimulation parameters as in Table 1). When impending serious cardiac arrhythmia is detected by the processing unit 50, 401, the processing unit 50, 401, may be further configured to automatically send or electronically communicate signals (impending-arrhythmia-warning signals) to the taVNS unit 30 to actuate the taVNS unit 30 immediately to start sending pre-determined electric stimuli to the auricular branch of vagus nerve via the vagus innervated auricular skin that the stimulating electrode 31 is in contact with (stimulation parameters as in Table 2). When the processing unit 50, 401, detects cessation of serious cardiac arrhythmia or cessation of impending serious cardiac arrhythmia, the processing unit 50, 401, may also be configured to send or electronically communicate signals (arrhythmia-cessation signals or impending-arrhythmia-cessation signals) to the taVNS unit 30 to modify or stop the electric stimuli to the vagus nerve via the stimulating electrode 31. This enables the chest-based automatic anti-arrhythmia system 200B to automatically initiate therapeutic intervention instantly. This can avoid the inevitable delay in traditional set-up where the ECG data being sent to a monitoring center and the monitoring center notifying healthcare provider 950 to arrange for therapeutic intervention.

In some embodiments, an arm-based automatic anti-arrhythmia system 200C (the system 200C) may comprise an arm-based ECG monitoring system 350, a taVNS unit 30 and a processing unit 50, 401. The processing unit 50, 401 is in electronic communication with the arm-based ECG monitoring system 350 and the taVNS unit 30. Example of an arm-based ECG monitoring system 350 includes a MUAC armband ECG from Ulster University, UK. Generally, an arm-based ECG monitoring system 350 may comprise a device that may be placed on an arm 911 of a wearer 900 with a plurality of electrodes 351 (arm-contacting ECG electrodes 351) attached to the skin of the wearer's 900 arm 911 (more commonly left upper arm), allowing it to record the electrical activity of the heart by picking up signals directly from the heart; essentially, the plurality of arm-contacting ECG electrodes 351 are positioned on the arm 911 to capture the heart's electrical signals. With the help of automatic ECG analysis algorithms, the processing unit 50, 401, is configured to analyze the ECG data to detect any serious cardiac arrhythmia and any impending serious cardiac arrhythmia, including AFib, AFL, SVT, VT, VF, etc., (such as via existing ECG analysis algorithms). Upon detection of serious cardiac arrhythmia, the processing unit 50, 401, may be further configured to automatically send signals (arrhythmia-warning signals) to the taVNS unit 30 to actuate the taVNS unit 30 immediately to start sending pre-determined electric stimuli to the auricular branch of vagus nerve via the vagus innervated auricular skin that the stimulating electrode 31 is in contact with (stimulation parameters as in Table 1). Similarly, upon detection of impending serious cardiac arrhythmia, the processing unit 50, 401, may be further configured to automatically send signals (impending-arrhythmia-warning signals) to the taVNS unit 30 to actuate the taVNS unit 30 immediately to start sending pre-determined electric stimuli to the auricular branch of vagus nerve via the vagus innervated auricular skin that the stimulating electrode 31 is in contact with (stimulation parameters as in Table 2). When the processing unit 50, 401, detects cessation of serious cardiac arrhythmia or cessation of impending serious cardiac arrhythmia, the processing unit 50, 401, may also be configured to send signals (arrhythmia-cessation signals or impending-arrythmia-cessation signals) to the taVNS unit 30 to modify or stop the electric stimuli to the vagus nerve via the stimulating electrode 31. This enables the arm-based automatic anti-arrhythmia system 200C to automatically initiate therapeutic intervention instantly. This can avoid the inevitable delay in traditional set-up where the ECG data being sent to a monitoring center and the monitoring center notifying healthcare provider 950 to arrange for therapeutic intervention.

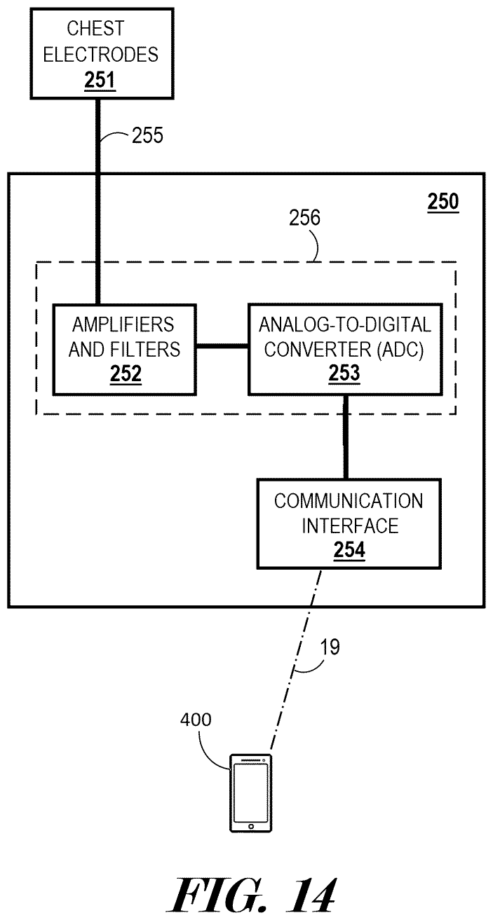

FIG. 14 depicts some example components of an example chest-based ECG monitoring system 250. For example, a chest-based ECG monitoring system 250 may include an ECG recording module 256 that may record and analyze the electrical activity of the wearer's 900 heart, such as to diagnose heart conditions and detect serious cardiac arrhythmias or impending serious cardiac arrhythmia. An ECG recording module 256 may include amplifiers and filters 252 that may pick up the electrical activity of the wearer's 900 heart via the electrodes (chest-contacting electrodes) 251 that may be in contact with the skin of the chest 910 of the wearer 900. An amplifier of amplifiers and filters 252 is responsible for amplifying the weak electrical signals received from the electrodes 251 via lead wires 255. The heart's electrical signals are typically very faint, often in the millivolt range. (The amplitude of ECG signals depends on the locations of ECG electrodes and could range from 10 microvolts to 5 millivolts. The amplitude also depends on different waves, including P, QRS or T waves.) The amplifier boosts these signals to a level that can be accurately recorded and displayed. Modern ECG machines use sophisticated amplifiers that minimize noise and ensure signal clarity. Filters of amplifiers and filters 252 are used to remove unwanted noise and interference from the electrical signals. Common sources of noise include muscle contractions, electrical interference from other devices, and movement artifacts. ECG machines use various filters, such as high-pass, low-pass, and notch filters, to clean the signals, ensuring that the resulting ECG trace is clear and interpretable. An ECG recording module 256 may include an analog-to-digital converter (ADC) 253 that may transform the analog electrical signals from the heart into digital data. This digital conversion is essential for processing, storing, and displaying the ECG data on a screen or print out. The ADC 253 ensures that the data is accurately digitized, preserving the integrity of the original signal. The ADC 253 may be in communication with a communication interface 254 which may enable electronic communication 19 (e.g., wired and/or wireless communication) of ECG data recorded by the chest-based ECG monitoring system 250 between the taVNS unit 30 and, optionally, another electronic device, such as a client device 400. Preferably, a communication interface 254 may comprise a radio that may operate via WiFi and/or Bluetooth communication standards. Additionally, a communication interface 254 may be configured as a network interface 53 described above so that it may operate on any wireless and/or wired electronic communication 19 protocol that a network interface 53 may use.

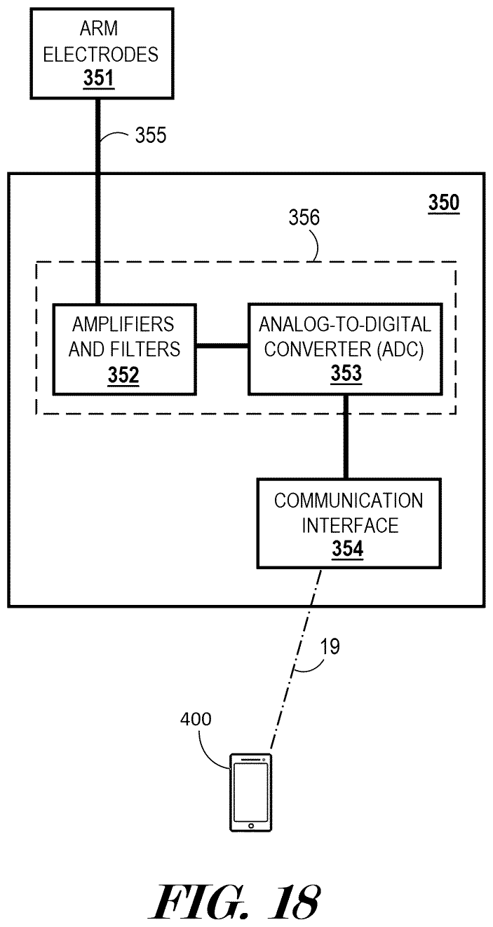

FIG. 18 depicts some example components of an example arm-based ECG monitoring system 350. For example, an arm-based ECG monitoring system 350 may include an ECG recording module 356 that may record and analyze the electrical activity of the wearer's 900 heart, such as to diagnose heart conditions and detect serious cardiac arrhythmias and impending serious cardiac arrhythmia. An ECG recording module 356 may include amplifiers and filters 352 that may pick up the electrical activity of the wearer's 900 heart via the electrodes 351 in contact with the skin of the arm 911 of the wearer 900. An amplifier of amplifiers and filters 352 is responsible for amplifying the weak electrical signals received from the electrodes 351 via lead wires 355. The heart's electrical signals are typically very faint, often in the millivolt range. (The amplitude of ECG signals depends on the locations of ECG electrodes and could range from 10 microvolts to 5 millivolts. The amplitude also depends on different waves, including P, QRS or T waves.) The amplifier boosts these signals to a level that can be accurately recorded and displayed. Modern ECG machines use sophisticated amplifiers that minimize noise and ensure signal clarity. Filters of amplifiers and filters 352 are used to remove unwanted noise and interference from the electrical signals. Common sources of noise include muscle contractions, electrical interference from other devices, and movement artifacts. ECG machines use various filters, such as high-pass, low-pass, and notch filters, to clean the signals, ensuring that the resulting ECG trace is clear and interpretable. An ECG recording module 356 may include an analog-to-digital converter (ADC) 353 that may transform the analog electrical signals from the heart into digital data. This digital conversion is essential for processing, storing, and displaying the ECG data on a screen or print out. The ADC 353 ensures that the data is accurately digitized, preserving the integrity of the original signal. The ADC 353 may be in communication with a communication interface 354 which may enable electronic communication 19 (e.g., wired and/or wireless communication) of ECG data recorded by the arm-based ECG monitoring system 350 between the taVNS unit 30 and, optionally, another electronic device, such as a client device 400. Preferably, a communication interface 354 may comprise a radio that may operate via WiFi and/or Bluetooth communication standards. In further embodiments, a communication interface 354 may be configured as a network interface 53 described above so that it may operate on any wireless and/or wired electronic communication 19 protocol that a network interface 53 may use.

The chest-based ECG monitoring system 250 typically has some disadvantages. For example, the Zio Patch Monitor can be used only for a short time (around 14 days). Assert-IQ ICM from Abbott can last for a longer time but it requires a surgery for placement. The arm-based ECG monitoring system 350 typically also has some disadvantages. The MUAC armband ECG monitoring from Ulster University, UK, can be used only for a short time (days to weeks) and could be interfered by movement artifacts or muscle artifacts.

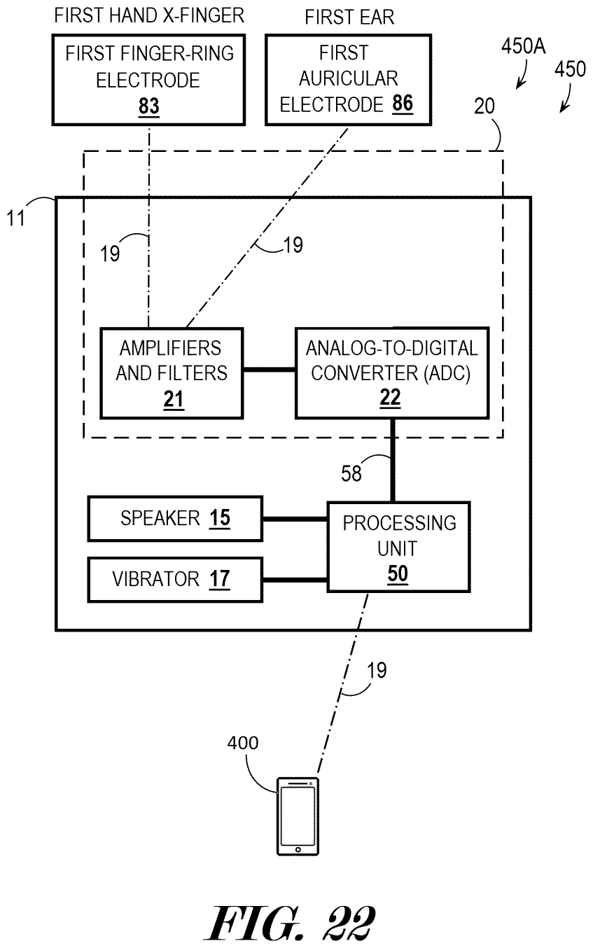

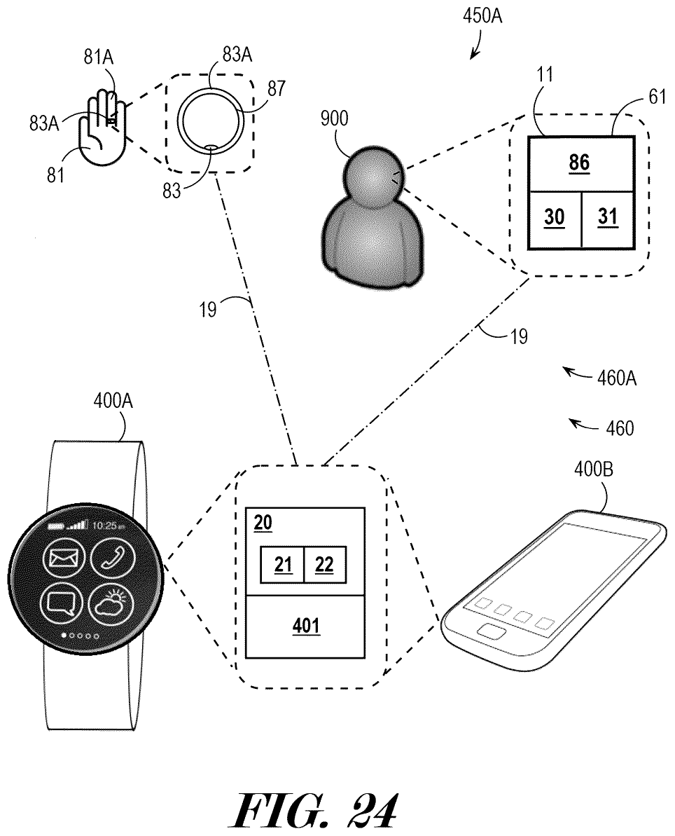

According to still a further aspect consistent with the principles of the invention, in some embodiments, a smart-ring electrocardiogram (ECG) monitoring system 450 may include a novel ear-finer ECG system 450A, a novel finger-finger ECG system 450B, a novel ear-toe ECG system 750A and a novel finger-toe ECG system 750B. In some embodiments, and referring to FIGS. 22, 24, the novel ear-finger ECG system 450A may comprise at least two wired or wireless ECG electrodes 83, 86. At least one of the ECG electrodes 83, 86, (for example a first finger-ring electrode 83) may be configured to be placed on an inner surface 87 of a first finger-ring 83A to be worn on a finger (for example an X-finger 81A) of a wearer's first hand 81. The first finger-ring electrode 83 may be configured to be located on the inner surface 87 of the first finger-ring 83A and the first finger-ring 83A is configured to be made of an elastic, adaptable and flexible material (such as silicone-type material) and the elastic, adaptable flexible material is configured to have appropriate size, elasticity, adaptability and flexibility such that the first finger-ring electrode 83 will snugly contact the skin of the X-finger 81A of the wearer's first hand 81 when the wearer 900 wears the first finger-ring 83A on the X-finger 81A of the wearer's first hand 81. The first finger-ring electrode 83 may be configured to be partially embedded in the inner surface 87 of the first finger-ring 83A with slight protrusion at the inner surface 87 of the first finger-ring 83A such that the first finger-ring electrode 83 will snugly contact the skin of the X-finger 81A of the wearer's first hand 81 when the first finger-ring 83A is worn on the X-finger 81A of the wearer's first hand 81. The ear-finger ECG system 450A further comprises at least another ECG electrode (for example a first auricular electrode 86) to be placed on a surface 27A of a first in-the-ear portion 27 of a first auricular housing structure 11 that is configured to be worn on or in a first ear 902 of the wearer 900. The first auricular housing structure 11 includes the first in-the-ear portion 27 that may be configured to be in contact an external ear canal 904 and a bowl-shaped area of tragus-concha 905, 906, 907, of the wearer's first ear 902 when the first in-the-ear portion 27 is inserted (or put in place) into the external ear canal 904 and bowl-shaped area of tragus-concha 905, 906, 907, of the wearer's first ear 902 (in a way essentially similar to when an in-the-ear portion 27 of a behind-the-ear-hearing-aid-style structure 11C is put in place.) The first auricular housing structure 11 may be selected from one of the following: an earbud-style structure 11A, a behind-the-ear-hearing-aid-style structure 11C, or a tubular-shaped structure 11B. The first in-the-ear portion 27 of the first auricular housing structure 11 may be configured to comprise an elastic, adaptable and flexible material (such as soft foam or soft silicone-type material.). The first in-the-ear portion 27 of the first auricular housing structure 11 may be configured to have appropriate size, elasticity, adaptability and flexibility such that the first auricular electrode 86 will snugly contact the skin of the wearer's external ear canal 904 and skin of bowl-shaped area of tragus-concha 905, 906, 907, of the wearer's first ear when the first in-the-ear portion 27 is put in place in the wearer's first ear 902. The first auricular electrode 86 is configured to be partially embedded in the surface 27A of the first in-the-ear portion 27 with slight protrusion at the surface 27A of the first in-the-ear portion 27 such that the first auricular electrode 86 will be naturally and snugly in contact with the skin of the external ear canal 904 and skin of bowl-shaped area of tragus-concha 905, 906, 907, of the wearer's first ear 902 when the first in-the-ear portion 27 is put into the external ear canal 904 and the bowl-shaped area of tragus-concha 905, 906, 907, of the wearer's first ear 902, similar to descriptions hereinbefore. The ear-finger ECG system 450A may further comprise an ECG recording module 20 that may be housed either within the first finger-ring 83A or within the first auricular housing structure 11. The first finger-ring electrode 83 and the first auricular electrode 86 may be configured to be wired or wireless (preferably wireless) ECG electrodes. The ECG recording module 20 may be configured to comprise a wired or wireless (preferably wireless) ECG amplifier. The first finger-ring electrode 83 and the first auricular electrode 86 are configured to be in wired or wireless (preferably wireless) communication with the ECG recording module 20 and the ECG recording module 20 is configured to record ECG data of the wearer 900 via the first finger-ring electrode 83 and the first auricular electrode 86. The ear-finger ECG system 450A further comprises a processing unit 50, 401, and the processing unit 50, 401 is configured to analyze the ECG data recorded by the ECG recording module 20 to detect presence or cessation of any serious cardiac arrhythmia or any impending serious cardiac arrhythmia (including AFib, AFL, SVT, VT and VF). The ear-finger ECG system 450A may further comprise a network interface 53, 406 and one of the following: a speaker 15, 404A, a vibrator 17, 404B, and a display screen 404C on a client device 400 of the wearer 900. The network interface 53, 406, may be in electronic communication with the processing unit 50, 401, the speaker 15, 404A, the vibrator 17, 404B, and the display screen 404C on a client device 400 of the wearer 900. When the processing unit 50, 401, detects presence of a serious cardiac arrhythmia, the processing unit 50, 401, is configured to send signals (arrhythmia-warning signals) to at least one of the following: the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. When the processing unit 50, 401, detects presence of an impending serious cardiac arrhythmia, the processing unit 50, 401, is configured to send signals (impending-arrhythmia-warning signals) to at least one of the following: the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401, may be configured to send visible notification to a display screen 404C of a client device 400 of the wearer's healthcare provider 950 to alert the healthcare provider 950 to take appropriate action.

As used herein, the term “auricular housing structure 11” refers to housing structures in or around the ear. Examples of auricular housing structures include an earbud-style structure 11A, a behind-the-ear-hearing-aid-style structure 11C, a tubular-shaped structure 11B and a taVNS housing structure 40 used for a taVNS unit 30. An auricular housing structure 11 may comprise as one of: an earbud style structure 11A, a tubular-shaped structure 11B, or a behind-the-ear-hearing-aid-style structure 11C. Also, as used herein, the term “in-the-ear portion 27” of an auricular housing structure 11 refers to a portion of the auricular housing structure 11 that is put inside (or into) an external ear canal 904 and a bowl-shaped area of tragus-concha 905, 906, 907, of an ear 902 when in use. The “in-the-ear portion 27” of an auricular housing structure 11 is essentially the same as an in-the-ear portion 27 of a behind-the-ear-hearing-aid-style structure 11C (a behind-the-ear-hearing-aid-style structure 11C typically includes an in-the-ear portion 27 and a behind-the-ear portion 26.) Additionally, as used herein, the term “horizontal portion 25” of an earbud-style structure 11A refers to the “body”, “nozzle” and “ear-tip” of the earbud-style structure 11A and the “nozzle” and “ear-tip” are typically inserted inside an external ear canal 904 while the “body” is typically sitting on bowl-shaped area of tragus-concha 905, 906, 907 (bowl-shaped area of tragus-concha 905, 906, 907, is located surrounding the opening of external ear canal 904) when the earbud-style structure 11A is put in use. Thus, the term “horizontal portion 25” of the earbud-style structure 11A is essentially the same as the “in-the-ear portion 27” of an auricular housing structure 11. A tubular-shaped structure 11B itself is a housing structure located inside an external ear canal 904 and thus is essentially part of the “in-the-ear portion 27” of an auricular housing structure 11. Additionally, as used herein, all of the self-installable self-removable housing structures (11, 11A, 11B, 11C, 83A, 84A, 73A) that are used to house auricular ECG electrodes (for example 12, 13, 88, 86), taVNS stimulating electrode 31, and smart ring electrodes (including first finger-ring electrode 83, second finger-ring electrode 84, and first toe-ring electrode 73) in this invention is referred collectively as “self-installable-housing structures” and these electrodes is referred collectively as “self-installable electrodes”. Examples of self-installable-housing structures may include the in-the-ear portion 27 of the auricular housing structure 11, an earbud-style structures 11A or hearing-aid-style structure 11B, 11C, and smart ring-type structures for fingers or toes (for example first finger-ring 83A, second finger-ring 84A, first toe-ring 73A). These self-installable housing structures can be self-installed and self-removed easily by the wearer (user) without any help from another person or a technologist and there is no need to pre-prepare the skin or use adhesive material to secure the electrodes.

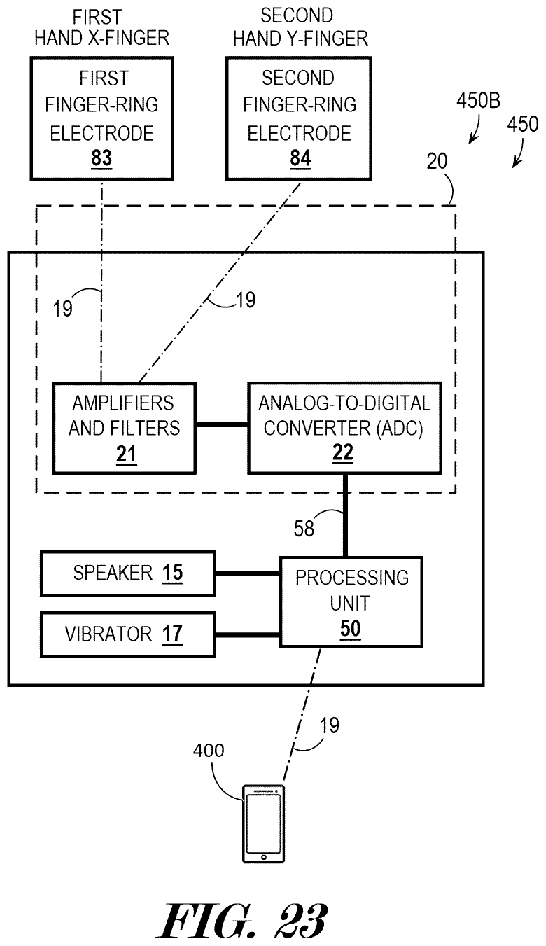

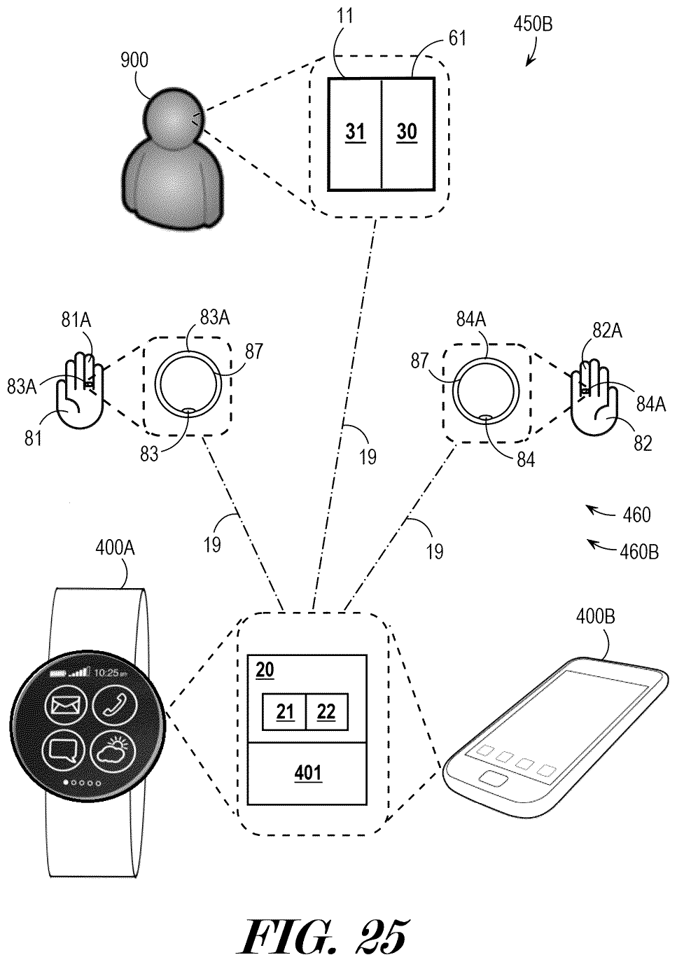

In some embodiments, and referring to FIGS. 23, 25, a novel finger-finger ECG system 450B may comprise at least two wired or wireless ECG electrodes 83, 84. At least one of the ECG electrodes 83, 84, (for example a first finger-ring electrode 83) is configured to be placed on an inner surface 87 of a first finger-ring 83A to be worn on a finger (for example an X-finger 81A) of the wearer's first hand 81. The finger-finger ECG system 450B further comprises at least another ECG electrode 83, 84, (for example a second finger-ring electrode 84) to be placed on an inner surface 87 of a second finger-ring 84A that is configured to be worn on a finger (for example a Y-finger 82A) of the wearer's second hand 82. The first finger-ring 83A may be configured to be made of elastic flexible adaptable material (for example silicone-type material) and the elastic flexible adaptable material is configured to have appropriate size, elasticity, adaptability and flexibility such that the first finger-ring 83A will snugly fit the X-finger 81A of the wearer's first hand 81 when the wearer wears the first finger-ring 83A on the X-finger 81A of the wearer's first hand 81. The first finger-ring electrode 83 may be configured to be partially embedded in the inner surface 87 of the first finger-ring 83A with slight protrusion at the inner surface 87 of the first finger-ring 83A such that the first finger-ring electrode 83 will naturally and snugly contact the skin of the X-finger 81A of the wearer's first hand 81 when the first finger-ring 83A is worn by the wearer 900. Likewise, the second finger-ring electrode 84 is configured to be placed on the inner surface 87 of the second finger-ring 84A and the second finger-ring 84A may be configured to be made of elastic flexible adaptable material and the elastic flexible adaptable material is configured to have appropriate size, flexibility, adaptability and elasticity such that the second finger-ring 84A will snugly fit the Y-finger 82A of the wearer's second hand 82 when the wearer wears the second finger-ring 84A on the Y-finger 82A of the wearer's second hand 82. The second finger-ring electrode 84 may be configured to be partially embedded in the inner surface 87 of the second finger-ring 84A with slight protrusion at the inner surface 87 of the second finger-ring 84A such that the second finger-ring electrode 84 will naturally and snugly contact the skin of the Y-finger 82A of the wearer's second hand 82 when the second finger-ring 84A is worn by the wearer 900. The first finger-ring electrode 83 and the second finger-ring electrode 84 may be configured as wired or wireless ECG electrodes (preferably wireless). The ECG recording module 20 may be configured to comprise wired or wireless ECG amplifier 21 (preferably wireless). The first finger-ring electrode 83 and the second finger-ring electrode 84 may be configured to be in wired or wireless (preferably wireless) communication with the ECG recording module 20 and the ECG recording module 20 is configured to record ECG data of the wearer via the first finger-ring electrode 83 and the second finger-ring electrode 84. The finger-finger ECG system 450B further comprises a processing unit 50, 401. The processing unit 50, 401, is configured to analyze the ECG data recorded by the ECG recording module 20 to assess the wearer's ECG profile and to detect presence or cessation of any serious cardiac arrhythmia and presence or cessation of any impending serious cardiac arrhythmia (including AFib, AFL, SVT, VT and VF). The finger-finger ECG system 450B may further comprise a network interface 53, 406, and one of the following: a speaker 15, 404A, a vibrator 17, 404B, and a display screen 404C on a client device 400 of the wearer 900. The network interface 53, 406, may be configured to be in electronic communication with the processing unit 50, 401, the speaker 15, 404A, the vibrator 17, 404B and the display screen 404C on a client device 400 of the wearer 900. When the processing unit 50, 401, detects presence of a serious cardiac arrhythmia or impending serious cardiac arrhythmia, the processing unit 50, 401, is configured to send signals to at least one of the following: the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401, may be configured to send visible notification to a display screen 404C of a client device 400 of the wearer's healthcare provider 950 to alert the healthcare provider 950 to take appropriate action.

According to still another aspect consistent with the principles of the present invention, a smart-ring automatic anti-arrhythmia system 460 may be configured as a novel ear-finger automatic anti-arrhythmia system 460A or a novel finger-finger automatic anti-arrhythmia system 460B. In some embodiments, and referring to FIG. 24, an ear-finger automatic anti-arrhythmia system 460A may be configured to comprise an ear-finger ECG system 450A, a transcutaneous auricular vagus verve stimulation (taVNS) unit 30 and a processing unit 50, 401. The ear-finger ECG system 450A and the taVNS unit 30 are as described hereinbefore. The ear-finger ECG system 450A comprises an ECG recording module 20 and at least two wired or wireless (preferably wireless) ECG electrodes, including a first finger-ring electrode 83 (to be housed in a first finger-ring 83A) and a first auricular electrode 86 (to be housed in an in-the-ear portion 27 of an auricular housing structure 11), similar to descriptions hereinbefore. The taVNS unit 30 of the ear-finger automatic anti-arrhythmia system 460A may be housed in a taVNS housing 40. Preferably, the taVNS housing 40 may be integrated with the auricular housing structure 11, similar to descriptions hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401, is in electronic communication (preferably wireless communication) with the transcutaneous auricular vagus verve stimulation (taVNS) unit 30 and the ear-finger ECG system 450A. The processing unit 50, 401, is configured to process the ECG data transmitted from the ear-finger ECG system 450A and the processing unit 50, 401, is configured to assess the wearer's ECG profile and to detect presence or cessation of a serious cardiac arrhythmia and presence or cessation of an impending serious cardiac arrhythmia (including AFib, AFL, SVT, VT and VF). When presence of at least one serious cardiac arrhythmia is detected by the processing unit 50, 401, the processing unit 50, 401, is configured to send signals (arrhythmia-warning signals) immediately to the taVNS unit 30. Upon receiving arrhythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with, similar to descriptions hereinbefore. (Examples for pre-determined electric stimulation parameters for the taVNS unit are shown on Table 1.) When presence of at least one impending serious cardiac arrhythmia is detected by the processing unit 50, 401, the processing unit 50, 401, is further configured to send signals (impending-arrhythmia-warning signals) immediately to the taVNS unit 30. Upon receiving impending-arrhythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with, similar to aforementioned descriptions. (Examples for pre-determined electric stimulation parameters for the taVNS unit are shown on Table 2.) When cessation of the at least one serious cardiac arrhythmia is detected by the processing unit 50, 401, the processing unit 50, 401, is further configured to send signals (arrhythmia-cessation signals) to the taVNS unit 30. Upon receiving arrhythmia-cessation signals from the processing unit 50, 401, the ta VNS unit 30 is configured to stop sending pre-determined electric stimuli. When cessation of the at least one impending serious cardiac arrhythmia is detected by the processing unit 50, 401, the processing unit 50, 401, is further configured to send signals (impending-arrhythmia-cessation signals) to the taVNS unit 30. Upon receiving impending-arrhythmia-cessation signals from the processing unit 50, 401, the taVNS unit 30 is configured to stop sending pre-determined electric stimuli. The ear-finger automatic anti-arrhythmia system 460A may further comprise a network interface 53, 406, and one of the following: a speaker 15, 404A, a vibrator 17, 404B, and a display screen 404C on a client device 400 of the wearer 900. The network interface 53, 406 is in electronic communication with the processing unit 50, 401, the speaker 15, 404A, the vibrator, 17, 404B, and the display screen 404C on a client device 400 of the wearer 900. When the processing unit 50, 401, detects presence of a serious cardiac arrhythmia or an impending serious cardiac arrhythmia, the processing unit 50, 401, is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401, may be configured to send visible notification to a display screen 404C of a client device 400 of the wearer's healthcare provider 950 to alert the healthcare provider 950 to take appropriate action.

In some embodiments, and referring to FIG. 25, a finger-finger automatic anti-arrhythmia system 460B may be configured to comprise a finger-finger ECG system 450B, a transcutaneous auricular vagus verve stimulation (taVNS) unit 30 and a processing unit 50, 401. The finger-finger ECG system 450B and the taVNS unit 30 are as described hereinbefore. The finger-finger ECG system 450B comprises an ECG recording module 20 and at least two wired or wireless (preferably wireless) ECG electrodes, including a first finger-ring electrode 83 (to be housed in a first finger-ring 83A) and a second finger-ring electrode 84 (to be housed in a second finger-ring 84A), similar to descriptions hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401, is in electronic communication (preferably wireless communication) with the taVNS unit 30 and the finger-finger ECG system 450B. The processing unit 50, 401, is configured to process the ECG data transmitted from the finger-finger ECG system 450B and the processing unit 50, 401, is configured to assess the wearer's ECG profile and to detect presence or cessation of a serious cardiac arrhythmia (including AFib, AFL, SVT, VT, VF) and presence or cessation of an impending serious cardiac arrhythmia. When presence of at least one serious cardiac arrhythmia is detected by the processing unit 50, 401, the processing unit 50, 401, is configured to send signals (arrhythmia-warning signals) immediately to the taVNS unit 30 and upon receiving the arrythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with (stimulation parameters as shown in Table 1). When presence of at least one impending serious cardiac arrhythmia is detected by the processing unit 50, 401, the processing unit 50, 401, is further configured to send signals (impending-arrhythmia-warning signals) immediately to the taVNS unit 30 and upon receiving the impending-arrythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with (stimulation parameters as shown in Table 2). When cessation of the at least one serious cardiac arrhythmia is detected by the processing unit 50, 401, the processing unit 50, 401, is further configured to send signals (arrhythmia-cessation signals) to the taVNS unit 30. Upon receiving the arrhythmia-cessation signals from the processing unit 50, 401, the taVNS unit 30 is configured to stop sending pre-determined electric stimuli. When cessation of the at least one impending serious cardiac arrhythmia is detected by the processing unit 50, 401, the processing unit 50, 401, is further configured to send signals (impending-arrhythmia-cessation signals) to the taVNS unit 30. Upon receiving the impending-arrhythmia-cessation signals from the processing unit 50, 401, the taVNS unit 30 is configured to stop sending pre-determined electric stimuli. The finger-finger automatic anti-arrhythmia system 460B may further comprise a network interface 53, 406, and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit detects presence of a serious cardiac arrhythmia or impending serious cardiac arrhythmia, the processing unit 50, 401, is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401, may be configured to send visible notification to a display screen 404C of a client device 400 of the wearer's healthcare provider 950 to alert the healthcare provider 950 to take appropriate action.

In some embodiments, and referring to FIG. 24, an ear-finger automatic anti-arrhythmia system 460A may be configured to comprise an ear-finger ECG system 450A, a taVNS unit 30 and a processing unit 50, 401. The processing unit 50, 401, is in electronic communication (preferably wireless communication) with the ear-finger ECG system 450A and the ta VNS unit 30. The ear-finger ECG system 450A and the taVNS unit 30 are as described hereinbefore. The ear-finger ECG system 450A comprises an ECG recording module 20 that is in electronic communication 19 (preferably wireless communication) with at least two ECG electrodes, including a first finger-ring electrode 83 and a first auricular electrode 86. The ECG recording module 20 is configured to record ECG data of the wearer via the first finger-ring electrode 83 and the first auricular electrode 86. The first finger-ring electrode 83 may be configured to be housed in a first finger-ring 83A to be worn on an X-finger 81A of the wearer's 900 first hand 81. The first finger-ring electrode 83 may be configured to be partially embedded in an inner surface 87 of the first finger-ring 83A with slight protrusion at the inner surface 87 of the first finger-ring 83A The first finger-ring 83A may be configured to comprise elastic flexible adaptable material and the elastic flexible adaptable material is configured to have appropriate size, elasticity, adaptability and flexibility such that the first finger-ring electrode 83 is snugly in contact with the X-finger 81A skin of the wearer's first hand 81 when the first finger-ring 83A is worn on the X-finger 81A of the wearer's first hand 81. The first auricular electrode 86 may be configured to be housed in an in-the-ear portion 27 of an auricular housing structure 11 and the in-the-ear portion 27 is configured to be put into an external ear canal 904 and a bowl-shaped area of tragus-concha 905, 906, 907, of a first ear 902 of the wearer 900 when in use. The taVNS unit 30 comprises a stimulating electrode 31 that may be configured to be housed in the in-the-ear portion 27 of the auricular housing structure 11. The first auricular electrode 86 and the stimulating electrode 31 may be configured to be partially embedded in a surface 27A of the in-the-ear portion 27 of the auricular housing structure 11 with slight protrusion at the surface 27A of the in-the-ear portion 27 of the auricular housing structure 11. The in-the-ear portion 27 may be configured to comprise elastic flexible adaptable material and the elastic flexible adaptable material is configured to have appropriate elasticity, adaptability and flexibility such that the first auricular electrode 86 and the stimulating electrode 31 are naturally and snugly in close contact with the skin of the external ear canal 904 and skin of bowl-shaped area of tragus-concha 905, 906, 907, of the wearer's first ear 902 when the in-the-ear portion 27 is put in place of the wearer's first ear 902. In this way, the stimulating electrode 31 of the taVNS unit 30 will be naturally contacting vagus innervated auricular skin since the skin of the external ear canal 904, tragus 905, concha 906, 907, all have rich vagal innervation. These features will allow easy self-installation and easy self-removal of the first finger-ring electrode 83 and the first auricular electrode 86 of the 460A system and the stimulating electrode 31 of the taVNS unit 30.

In some embodiments, and referring to FIG. 25, the finger-finger automatic anti-arrhythmia system 460B comprises a finger-finger ECG system 450B, a taVNS unit 30 and a processing unit 50, 401. The processing unit 50, 401 is in electronic communication (preferably wireless communication) with the finger-finger ECG system 450B and the taVNS unit 30. The finger-finger ECG system 450B and the taVNS unit 30 are as described hereinbefore. The finger-finger ECG system 450B comprises an ECG recording module 20 that may be in electronic communication 19 (preferably wireless communication) with at least two ECG electrodes, including a first finger-ring electrode 83 and a second finger-ring electrode 84. The ECG recording module 20 may be configured to record ECG data of the wearer via the first finger-ring electrode 83 and the second finger-ring electrode 84. The first finger-ring electrode 83 may be configured to be housed in a first finger-ring 83A to be worn on an X-finger 81A of the wearer's 900 first hand 81, while the second finger-ring electrode 84 may be configured to be housed in a second finger-ring 84A to be worn on a Y-finger 82A of the wearer's 900 second hand 82. The first finger-ring electrode 83 may be configured to be partially embedded in an inner surface 87 of the first finger-ring 83A with slight protrusion at the inner surface 87 of the first finger-ring 83A. The first finger-ring 83A may be configured to comprise elastic flexible adaptable material and the elastic flexible adaptable material may be configured to have appropriate size, elasticity, adaptability and flexibility such that the first finger-ring electrode 83 is naturally and snugly contacting the skin of the X-finger 81A of the wearer's first hand 81 when the first finger-ring 83A is worn on the X-finger 81A of the wearer's first hand 81. The second finger-ring electrode 84 may be configured to be partially embedded in an inner surface 87 of the second finger-ring 84A with slight protrusion at the inner surface 87 of the second finger-ring 84A. The second finger-ring 84A may be configured to comprise elastic flexible adaptable material and the elastic flexible adaptable material is configured to have appropriate size, elasticity, adaptability and flexibility such that the second finger-ring electrode 84 is snugly naturally contacting the skin of the Y-finger 82A of the wearer's second hand 82 when the second finger-ring 84A is worn on the Y-finger 82A of the wearer's second hand 82. These features will allow easy self-installation and easy self-removal of the first finger-ring electrode 83 and the second finger-ring electrode 84 of the 460B system.

Now referring to FIG. 22 that shows components of an ear-finger ECG system 450A of a smart-ring ECG monitoring system 450. The ear-finger ECG system 450A comprises an ECG recording module 20 that may include a first finger-ring electrode 83 and a first auricular electrode 86 in electronic communication 19 (preferably wireless communication) with an amplifier and filter 21. The ear-finger ECG system 450A further comprises an analog-to-digital converter (ADC) 22, a processing unit 50, a speaker 15, a vibrator 17, and a client device 400 in wireless electronic communication 19 with the system 450A, etc., similar to descriptions hereinbefore. Now referring to FIG. 23 that shows components of a finger-finger ECG system 450B of a smart-ring ECG monitoring system 450. The finger-finger ECG system 450B comprises an ECG recording module 20 that may include a first finger-ring electrode 83 and a second finger-ring electrode 84 in electronic communication 19 (preferably wireless communication) with an amplifier and filter 21. The finger-finger ECG system 450B further comprises an analog-to-digital converter (ADC) 22, a processing unit 50, a speaker 15, a vibrator 17, and a client device 400 in wireless electronic communication 19 with the system 450B, etc., similar to descriptions hereinbefore.

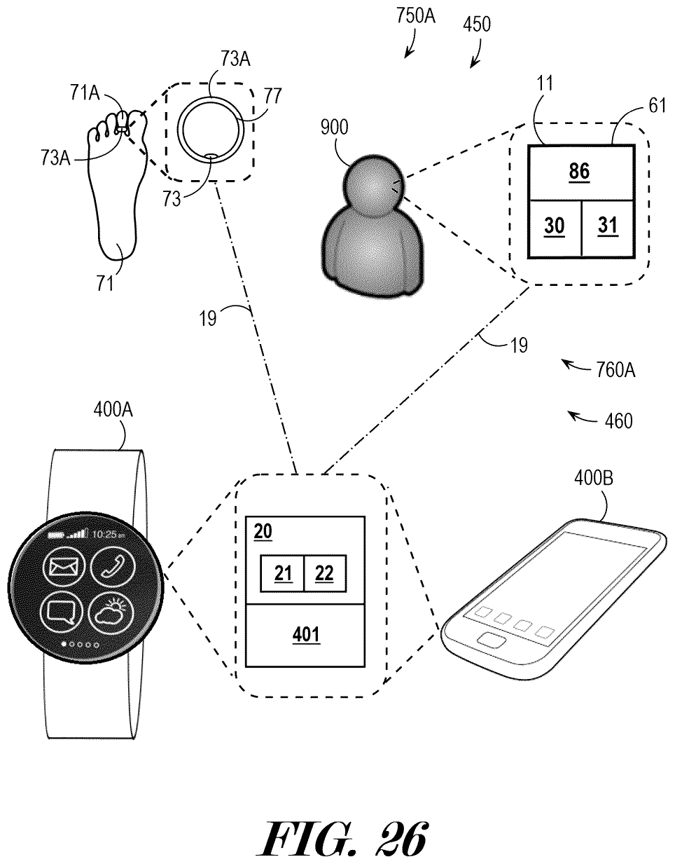

In alternative embodiments, and referring to FIGS. 26 & 27, a smart-ring ECG monitoring system 450 may be configured as an ear-toe ECG system 750A or a finger-toe ECG system 750B. In other embodiments, a smart-ring automatic anti-arrhythmia system 460 may be configured as an ear-toe automatic anti-arrhythmia system 760A or a finger-toe automatic anti-arrhythmia system 760B. In some embodiments, and referring to FIG. 26, an ear-toe ECG system 750A may comprise at least two wired or wireless ECG electrodes 73, 86. At least one of the ECG electrodes (for example a first toe-ring electrode 73) is configured to be placed on an inner surface 77 of a first toe-ring 73A to be worn on a toe (for example an X-toe 71A) of a wearer's first foot 71. The first toe-ring electrode 73 is configured to be located on an inner surface 77 of the first toe-ring 73A and the first toe-ring 73A is configured to be made of an elastic adaptable and flexible material (such as silicone-type material) and the elastic adaptable flexible material is configured to have appropriate size, elasticity, adaptability and flexibility such that the first toe-ring 73A will snugly contact the skin of the X-toe 71A of the wearer's first foot 71 when the wearer wears the first toe-ring 73A on the X-toe 71A of the wearer's first foot 71. The first toe-ring electrode 73 is configured to be partially embedded in the inner surface 77 of the first toe-ring 73A with slight protrusion at the inner surface 77 of the first toe-ring 73A such that the first toe-ring electrode 73 will naturally snugly contact the skin of the X-toe 71A of the wearer's first foot 71 when the first toe-ring 73A is worn on the X-toe 71A of the wearer's first foot 71. Thus, the first toe-ring electrode 73 can be easily self-installed and self-removed by the wearer (user) and there is no need to have a technologist to perform installation or removal of the first toe-ring electrode 73. The ear-toe ECG system 750A further comprises at least another ECG electrode (for example a first auricular electrode 86) to be placed on a surface 27A of an in-the-ear portion 27 of an auricular housing structure 11 that is configured to be worn on or in an ear 902 of the wearer 900. The auricular housing structure 11 includes an in-the-ear portion 27 that is configured to be in contact with an external ear canal 904 and a bowl-shaped area of tragus-concha 905, 906, 907, of an ear 902 of the wearer when the in-the-ear portion 27 is put in place into a the external ear canal 904 and the bowl-shaped area of tragus-concha 905, 906, 907, of the wearer's ear 902. The auricular housing structure 11 may be selected from one of the following: an earbud-style structure 11A, a behind-the-ear-hearing-aid-style structure 11C, or a tubular-shaped structure 11B. Optionally, a taVNS housing 40 may be configured to be integrated into or with the auricular housing structure 11. The in-the-ear portion 27 of the auricular housing structure 11 is configured to comprise an elastic, adaptable and flexible material (such as soft foam or soft silicone-type material.). The first auricular electrode 86 is configured to be partially embedded in a surface 27A of the in-the-ear portion 27 with slight protrusion at the surface 27A of the in-the-ear portion 27 of the auricular housing structure 11. The in-the-ear portion 27 of the auricular housing structure 11 is configured to have appropriate size, elasticity, adaptability and flexibility such that the in-the-ear portion 27 will naturally snugly fill the interior of the external ear canal 904 and the bowl-shaped area of tragus-concha 905, 906, 907, of the wearer's ear 902 when the in-the-ear portion 27 is put in place into the external ear canal 904 and the bowl-shaped area of tragus-concha 905, 906, 907, of the wearer's ear 902 and such that the first auricular electrode 86 will naturally snugly contact the skin of the external ear canal 904 or skin of the bowl-shaped area of tragus-concha 905, 906, 907, of the wearer's ear 902 when the in-the-ear portion 27 is put in place. Installing and removing the first auricular electrode 86 will be as easy as inserting and removing the in-the-ear portion 27 from the wearer's ear 902. This feature of easy self-installation and easy self-removal will be very convenient for the wearer. The ear-toe ECG system 750A may further comprise an ECG recording module 20 that may be housed either within the first toe-ring 73A or within the auricular housing structure 11. The first toe-ring electrode 73 and the first auricular electrode 86 are configured to be wired or wireless ECG electrodes (preferably wireless). The ECG recording module 20 is configured to comprise a wired or wireless ECG amplifier 21 (preferably wireless). The first toe-ring electrode 73 and the first auricular electrode 86 are configured to be in wired or wireless (preferably wireless) communication with the ECG recording module 20 and the ECG recording module 20 is configured to record ECG data of the wearer via the first toe-ring electrode 73 and the first auricular electrode 86. The processing unit 50, 401, is configured to analyze the ECG data recorded by the ECG recording module 20 to detect presence or cessation of a serious cardiac arrhythmia (including AFib, AFL, SVT, VT and VF). The processing unit 50, 401, is further configured to analyze the ECG data recorded by the ECG recording module 20 to detect presence or cessation of an impending serious cardiac arrhythmia. The ear-toe ECG system 750A may further comprise a network interface 53, 406, and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit 50, 401, detects presence of a serious cardiac arrhythmia, the processing unit 50, 401, is configured to send signals to at least one of the following: the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401, may be configured to send visible notification to a display screen 404C of a client device 400 of the wearer's healthcare provider 950 to alert the healthcare provider 950 to take appropriate action. Similarly, when the processing unit 50, 401, detects presence of an impending serious cardiac arrhythmia, the processing unit 50, 401, is configured to send signals to at least one of the following: the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401, may be configured to send visible notification to a display screen 404C of a client device 400 of the wearer's healthcare provider 950 to alert the healthcare provider 950 to take appropriate action.

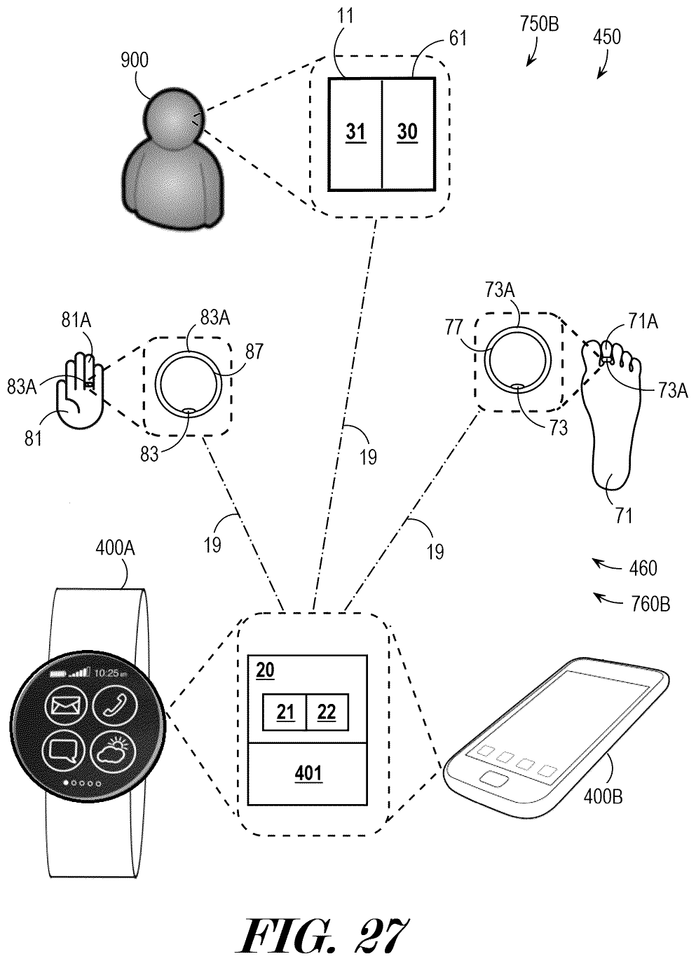

Referring to FIG. 27, in some embodiments, a finger-toe ECG system 750B may comprise at least two wired or wireless ECG electrodes. At least one of the ECG electrodes (for example a first finger-ring electrode 83) is configured to be placed on an inner surface 87 of a first finger-ring 83A to be worn on a finger (for example an X-finger 81A) of the wearer's first hand 81. The finger-toe ECG system 750B further comprises at least another ECG electrode (for example a first toe-ring electrode 73) to be placed on an inner surface 77 of a first toe-ring 73A that is configured to be worn on a toe (for example an X-toe 71A) of the wearer's first foot 71. The first finger-ring 83A may be configured to be made of elastic flexible adaptable material (for example silicone-type material) and the elastic flexible adaptable material is configured to have appropriate size, elasticity, adaptability and flexibility such that the first finger-ring 83A will snugly fit the X-finger 81A of the wearer's first hand 81 when the wearer wears the first finger-ring 83A on the X-finger 81A of the wearer's first hand 81. The first finger-ring electrode 83 is configured to be partially embedded in the inner surface 87 of the first finger-ring 83A with slight protrusion at the inner surface 87 of the first finger-ring 83A such that the first finger-ring electrode 83 will naturally snugly contact the skin of the X-finger 81A of the wearer's first hand 81 when the first finger-ring 83A is worn by the wearer 900. Likewise, the first toe-ring electrode 73 is configured to be placed on the inner surface 77 of the first toe-ring 73A and the first toe-ring 73A is configured to be made of elastic flexible adaptable material and the elastic flexible adaptable material is configured to have appropriate size, flexibility, adaptability and elasticity such that the first toe-ring electrode 73 will snugly fit the X-toe 71A of the wearer's first foot 71 when the wearer wears the first toe-ring 73A on the X-toe 71A of the wearer's first foot 71. The first toe-ring electrode 73 is configured to be partially embedded in the inner surface 77 of the first toe-ring 73A with slight protrusion at the inner surface 77 of the first toe-ring 73A such that the first toe-ring electrode 73 will naturally snugly contact the skin of the X-toe 71A of the wearer's first foot 71 when the first toe-ring 73A is worn by the wearer. The first finger-ring electrode 83 and the first toe-ring electrode 73 are configured as wired or wireless ECG electrodes (preferably wireless). The ECG recording module 20 is configured to comprise wired or wireless ECG amplifier (preferably wireless). The first finger-ring electrode 83 and the first toe-ring electrode 73 are configured to be in wired or wireless communication (preferably wireless) with the ECG recording module 20 and the ECG recording module 20 is configured to record ECG data of the wearer via the first finger-ring electrode 83 and the first toe-ring electrode 73. The processing unit 50, 401, is configured to analyze the ECG data recorded by the ECG recording module 20 to assess the wearer's ECG profile and to detect presence or cessation of any serious cardiac arrhythmia (including AFib, AFL, SVT, VT, and VF) and presence or cessation of any impending serious cardiac arrhythmia. The finger-toe ECG system 750B may further comprise a network interface 53, 406, and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit 50, 401, detects presence of a serious cardiac arrhythmia, the processing unit 50, 401, is configured to send signals to at least one of the following: the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer to take appropriate action. Additionally, the processing unit may be configured to send visible notification to a display screen 404C of a client device 400 of the wearer's healthcare provider 950 to alert the healthcare provider 950 to take appropriate action. Furthermore, when the processing unit 50, 401, detects presence of an impending serious cardiac arrhythmia, the processing unit 50, 401, is configured to send signals to at least one of the following: the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer to take appropriate action. Additionally, the processing unit 50, 401, may be configured to send visible notification to a display screen 404C of a client device 400 of the wearer's healthcare provider 950 to alert the healthcare provider 950 to take appropriate action.

Referring to FIG. 26, in some embodiments, an ear-toe automatic anti-arrhythmia system 760A may be configured to comprise an ear-toe ECG system 750A, a transcutaneous auricular vagus verve stimulation (taVNS) unit 30 and a processing unit 50, 401. The ear-toe ECG system 750A and the taVNS unit 30 are as described hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401, is in electronic communication (preferably wireless communication) with the transcutaneous auricular vagus nerve stimulation (taVNS) unit 30 and the ear-toe ECG system 750A. The processing unit 50, 401, is configured to process the ECG data transmitted from the ear-toe ECG system 750A and the processing unit 50, 401, is configured to assess the wearer's ECG profile and to detect presence or cessation of a serious cardiac arrhythmia or an impending serious cardiac arrhythmia (including AFib, AFL, SVT, VT, and VF). When at least one serious cardiac arrhythmia is detected by the processing unit 50, 401, the processing unit 50, 401, is configured to send signals (arrhythmia-warning signals) to the taVNS unit to prompt the taVNS unit to start sending pre-determined electric stimulation to the vagus innervated auricular skin to which the stimulating electrode 31 of the taVNS unit 30 is in contact with, similar to descriptions hereinbefore. (Examples for pre-determined electric stimulation parameters for the taVNS unit for serious cardiac arrhythmia are shown on Table 1.) When cessation of the at least one serious cardiac arrhythmia is detected by the processing unit 50, 401, the processing unit 50, 401, is further configured to send signals (arrhythmia-cessation signals) to the taVNS unit to cause the taVNS unit to stop sending pre-determined electric stimuli. When at least one impending serious cardiac arrhythmia is detected by the processing unit 50, 401, the processing unit 50, 401, is further configured to send signals (impending-arrhythmia-warning signals) to the taVNS unit 30 to prompt the taVNS unit 30 to start sending pre-determined electric stimulation to the vagus innervated auricular skin to which the stimulating electrode 31 of the taVNS unit 30 is in contact with, similar to descriptions hereinbefore. (Examples for pre-determined electric stimulation parameters for the taVNS unit for impending serious cardiac arrhythmia are shown on Table 2.) When cessation of the at least one impending serious cardiac arrhythmia is detected by the processing unit 50, 401, the processing unit 50, 401, is further configured to send signals impending-arrhythmia-cessation signals) to the taVNS unit 30 to cause the taVNS unit 30 to stop sending pre-determined electric stimuli. The ear-toe automatic anti-arrhythmia system 760A may further comprise a network interface 53, 406, and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit 50, 401, detects presence of a serious cardiac arrhythmia, the processing unit 50, 401, is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401, may be configured to send visible notification to a display screen 404C of a client device 400 of the wearer's healthcare provider 950 to alert the healthcare provider 950 to take appropriate action. Additionally, when the processing unit 50, 401, detects presence of an impending serious cardiac arrhythmia, the processing unit 50, 401, is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer to take appropriate action. Furthermore, the processing unit 50, 401, may be configured to send visible notification to a display screen 404C of a client device 400 of the wearer's healthcare provider 950 to alert the healthcare provider 950 to take appropriate action.

In some embodiments, and referring to FIG. 27, a finger-toe automatic anti-arrhythmia system 760B may be configured to comprise a finger-toe ECG system 750B, a transcutaneous auricular vagus verve stimulation (taVNS) unit 30 and a processing unit 50, 401. The finger-toe ECG system 750B and the taVNS unit 30 are as described hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401, is in electronic communication (preferably wireless communication) with the taVNS unit 30 and the finger-toe ECG system 750B. The processing unit 50, 401, is configured to process the ECG data transmitted from the finger-toe ECG system 750B and the processing unit 50, 401, is configured to assess the wearer's ECG profile and to detect presence or cessation of a serious cardiac arrhythmia or an impending serious cardiac arrhythmia (including AFib, AFL, SVT, VT and VF). When at least one serious cardiac arrhythmia is detected by the processing unit 50, 401, the processing unit 50, 401, is further configured to send signals (arrhythmia-warning signals) to the taVNS unit 30 to cause the taVNS unit 30 to start sending pre-determined electric stimulation to the vagus innervated auricular skin to which the stimulating electrode 31 of the taVNS unit 30 is in contact with (stimulating parameters as shown in Table 1). When at least one impending serious cardiac arrhythmia is detected by the processing unit 50, 401, the processing unit 50, 401, is further configured to send signals (impending-arrhythmia-warning signals) to the taVNS unit to cause the taVNS unit to start sending pre-determined electric stimulation to the vagus innervated auricular skin to which the stimulating electrode 31 of the taVNS unit 30 is in contact with (stimulating parameters as shown in Table 2). When cessation of the at least one serious cardiac arrhythmia is detected by the processing unit, the processing unit is further configured to send signals (arrhythmia-cessation signals) to the taVNS unit to cause the taVNS unit to stop sending pre-determined electric stimuli. When cessation of the at least one impending serious cardiac arrhythmia is detected by the processing unit, the processing unit is further configured to send signals (impending-arrhythmia-cessation signals) to the taVNS unit to cause the taVNS unit to stop sending pre-determined electric stimuli. The finger-toe automatic anti-arrhythmia system 760B may further comprise a network interface 53, 406, and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit 50, 401, detects presence of a serious cardiac arrhythmia or presence of an impending serious cardiac arrhythmia, the processing unit 50, 401, is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. The processing unit 50, 401, may be further configured to send visible notification to a display screen 404C of a client device 400 of the wearer's healthcare provider 950 to alert the healthcare provider 950 to take appropriate action.

For simplicity purpose, a block diagram (and its descriptions) for an ear-toe ECG system 750A is omitted although it is essentially similar to the block diagram for an ear-finger ECG system 450A (FIG. 22) and the descriptions of the functions of all of the components are essentially the same as those for the ear-finger ECG system 450A. Likewise, a block diagram (and its descriptions) for a finger-toe ECG system 750B is omitted for simplicity, although it is essentially similar to the block diagram for a finger-finger ECG system 450B (FIG. 23) and the descriptions of the functions of all of the components are essentially the same as those for the finger-finger ECG system 450B.

It should be noted that the smart-ring ECG monitoring systems 450 (including the ear-finger ECG system 450A, the finger-finger ECG system 450B, the ear-toe ECG system 750A and the finger-toe ECG system 750B) contain self-installable and self-removable ECG electrodes 73, 83, 84, 86, and these systems are wearable, due to the set-ups and features of the self-installable-housing structures 11, 83A, 84A, 73A (features including being made with flexible elastic adaptable material and electrodes being partially embedded in the surface with slight protrusion at the surface of the electrode-housing structures 11, 83A, 84A, 73A). Likewise, the ear-finger automatic anti-arrhythmia system 460A and a taVNS unit 30 also contains self-installable ECG electrodes 83, 86, and self-installable taVNS stimulating electrode 31 and the system is fully wearable, self-installable and self-removable. Similarly, the finger-finger automatic anti-arrhythmia system 460B and a taVNS unit 30 also contains self-installable ECG electrodes 83, 84, and self-installable taVNS stimulating electrode 31 and these electrodes are fully self-installable and self-removable. Similarly, the ear-toe automatic anti-arrhythmia system 760A and the finger-toe automatic anti-arrhythmia system 760B also contain self-installable and self-removable electrodes 73, 83, 86, 31. These are due to the set-ups and features of the self-installable-housing structures 11, 40, 83A, 73A. These systems are all wearable, self-installable, self-removable and the wearer can be freely ambulatory. Additionally, the aforementioned auricular ECG monitoring system 100 (including the single-ear auricular ECG system 100A and the dual-ear auricular ECG system 100B) and the auricular automatic anti-arrhythmia system 200A all contain self-installable self-removable electrodes 12, 13, 88, 31, that are housed an in in-the-ear portion 27 of an auricular housing structure 11 (including 11A, 11B, 11C). The in-the-ear portion 27 of the auricular housing structure 11 is another excellent example of a self-installable-housing structure that can be self-installed and self-removed by the wearer due to the features (made with elastic flexible adaptable material and electrodes partially embedded in the surface with slight protrusion at the surface of the electrode-housing structures 11, 11A, 11B, 11C) and setups of the electrode-housing structures. The in-the-ear portion 27 of the auricular housing structures 11, 11A, 11B, 11C, and the smart-ring housing 83A, 84A, 73A, will be collectively referred as “self-installable-housing structures” that can be self-installed and self-removed by the wearer (user). The auricular ECG monitoring system 100, 100A and 100B and the auricular automatic anti-arrhythmia system 200A are also excellent examples of wearable self-installable self-removable systems.

As a vital component of an automatic anti-arrhythmia system (200A, 200B, 200C, 460A, 460B, 760A, 760B), a taVNS unit 30 may comprise any device that is able to provide transcutaneous auricular vagus nerve stimulation to a user's body. As an example, and referring to FIG. 13, a taVNS unit 30 may comprise a microcontroller 33 that may be in communication with a pulse generator 34, voltage regulator 35, voltage transformer 36, amplifier 37, and buffer 38, and that may be configured to generate taVNS electrical stimuli (electrical impulses) that may be delivered to vagus nerve innervated auricular skin via a stimulating electrode 31. Examples of the parameters of a taVNS electrical stimuli are shown in Table 1 and Table 2. Optionally, a taVNS unit 30 may comprise a communication interface 42 which may enable electronic communication 19 (e.g., wired and/or wireless communication) between the taVNS unit 30 and another electronic device, such as a client device 400, an auricular ECG monitoring system 100, 100A, 100B, a chest-based ECG monitoring system 250, an arm-based ECG monitoring system 350 and smart ring ECG monitoring systems 450A, 450B, 750A, 750B. Preferably, a communication interface 42 may comprise a radio that may operate via WiFi and/or Bluetooth communication standards. In further embodiments, a communication interface 42 may be configured as a network interface 53 described above so that it may operate on any wireless and/or wired electronic communication 19 protocol that a network interface 53 may use.

One or more components (31, 32, 33, 34, 35, 36, 37, 38, 42) of a taVNS unit 30 may be contained in a taVNS housing 40 and may be in electronic communication via a local interface 43. A taVNS housing 40 may be configured in any size and shape, and may be made from or comprise soft foam, soft silicone type material, plastic, elastomer, or any other material used in the field of personal medical devices. In some embodiments, a taVNS housing 40 may be configured as one of the following: an earbud-style structure 61, a tubular-shaped structure 62 or a behind-the-ear-hearing-aid-style structure 63. Preferably, for an auricular automatic anti-arrhythmia system 200A, a taVNS housing 40 may be coupled directly to or integrated with an auricular housing structure 11. Optionally, a taVNS housing 40 may be a standalone housing that may be remote from an auricular housing structure 11 (for auricular automatic anti-arrhythmia system 200A). For a chest-based automatic anti-arrhythmia system 200B or an arm-based automatic anti-arrhythmia system 200C, a taVNS housing 40 will be a standalone housing and the taVNS unit 30 may be in wireless communication with the chest-based electrocardiogram (ECG) monitoring system 250 or the arm-based ECG monitoring system 350. Alternatively, an auricular housing structure 11 may be used for housing purpose for the taVNS unit 30 in the chest-based automatic anti-arrhythmia system 200B or the arm-based automatic anti-arrhythmia system 200C. Likewise, for a smart-ring automatic anti-arrhythmia system 460 (including 460A, 460B, 760A, 760B), a taVNS housing 40 may be a standalone housing and the taVNS unit 30 may be in wireless communication with a smart-ring ECG monitoring system 450 (including 450A, 450B, 750A, 750B). Optionally, an auricular housing structure 11 may be used for housing purpose for the taVNS unit 30 in the chest-based (200B) and arm-based (200C) automatic anti-arrhythmia system and smart-ring automatic anti-arrhythmia systems (460A, 460B, 760A, 760B). In some embodiments, the taVNS unit 30 may include a stimulating electrode 31 which may be built within the taVNS housing 40 of the taVNS unit 30 (so that the taVNS housing 40 and stimulating electrode 31 may form a single unit). In alternative embodiments, a stimulating electrode 31 may be connected with the taVNS housing 40 through a wire 32 (so that the stimulating electrode 31 may be remote from the taVNS housing 40 of the taVNS unit 30).

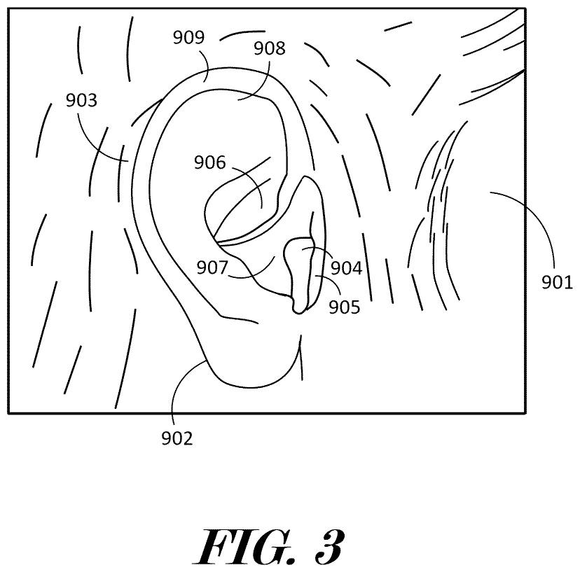

The stimulating electrode 31 of an automatic anti-arrhythmia system 200A, 200B, 200C, 460A, 460B, 760A, 760B, may be attached to vagus nerve innervated auricular skin. Vagus nerve innervated auricular skin that the stimulating electrode 31 may be attached to may include external ear canal 904, tragus 905, cymba-concha 906, and cavum-concha 907. These areas are innervated by the auricular branch of the vagus nerve. Optionally, the ta VNS unit 30 itself is also attached to the tragus 905, cymba-concha 906, cavum-concha 907, external ear canal 904, or a peri-auricular area 903 where a behind-the-ear-hearing-aid is typically located (FIG. 3). The tragus 905, concha region 906, 907, and external ear canal 904 are inherently stable for attachment of a taVNS unit 30 on a long-term basis. A behind-the-ear-hearing-aid-style set up is also inherently stable for attachment of a taVNS unit 30.

In some embodiments, an automatic anti-arrhythmia system 200A, 200B, 200C, 460A, 460B, 760A, 760B, may comprise a speaker 15, 404A or a vibrator 17, 404B, which may be configured to generate an audible or tactile notification when presence of a serious cardiac arrhythmia or presence of an impending serious cardiac arrhythmia is detected by a processing unit 50, 401. The speaker 15, 404A, may comprise a buzzer, a piezoelectric sound producing device, a dielectric elastomer sound producing device, a buzzer, a moving coil loudspeaker, an electrostatic loudspeaker, an isodynamic loudspeaker, a piezo-electric loudspeaker, or any other device capable of producing one or more sounds. The vibrator 17, 404B, may comprise a weight that may be rapidly moved by a long life brushless (BLDC) vibration motor, a coin or pancake vibration motor, an encapsulated vibration motor, an enclosed vibration motor, a pager motor, an eccentric rotating mass (ERM) motor, a linear resonant actuator (LRA), a printed circuit board (PCB) mounted vibration motor, or any other electrical device capable of producing a series of rapid and repeated movements.

Certain external ear 902 areas, especially the tragus 905, concha 906, 907, and external ear canal 904, have rich innervation of the auricular branch of vagus nerve. The external ear 902 is very unique anatomically because the external ear 902 is the only location of the human body where the vagus nerve reaches the body surface (the skin). The vagus nerve is also very unique physiologically because it is the main component of the parasympathetic nervous system. The vagus nerve regulates many very important human physiological functions, including heart rate, blood pressure, respiration, digestion, metabolism and stress response, etc. Thus, the external ear 902 is the best location for attachment of a taVNS unit 30 of an automatic anti-arrhythmia system 200A, 200B, 200C, 460A, 460B, 760A, 760B.

With the novel integration of a taVNS unit 30 along with an ECG monitoring system (including one of: an auricular ECG monitoring system 100, a chest-based ECG monitoring system 250, or an arm-based ECG monitoring system 350, smart ring ECG monitoring systems 450A, 450B, 750A 750B), an automatic anti-arrhythmia system 200A, 200B, 200C, 460A, 460B, 760A, 760B, enables automatic therapeutic action to be initiated instantly when a serious cardiac arrhythmia or an impending serious cardiac arrhythmia is detected. These ECG monitoring systems can record ECG data for long-term monitoring. The taVNS unit 30 of an automatic anti-arrhythmia system 200A, 200B, 200C, 460A, 460B, 760A, 760B, may comprise a stimulating electrode 31 which can give pre-determined electric current or electric stimuli transcutaneously in a way similar to transcutaneous electrical nerve stimulation (TENS) similar to existing taVNS and TENS devices. The stimulus parameters of the electric stimulation or electric stimuli (including strength, pattern, duration, interval, frequency and timing etc.) are pre-determined and could be adjusted for various types of cardiac arrhythmia. Examples of the stimulus parameters of the electric stimuli is listed in Table 1 and Table 2.

In some embodiments, an automatic anti-arrhythmia system 200A, 200B, 200C, 460A, 460B, 760A, 760B, may comprise an auricular housing structure 11 that may include an in-the-ear portion 27 and the in-the-ear portion 27 may be placed inside an external ear canal 904 and a bowl-shaped area of tragus-concha 905, 906, 907, when in use, similar to most commonly available hearing aids. Since cardiac arrhythmia affects a lot of old-aged people and many of them already need hearing aids, an auricular automatic anti-arrhythmia system 200A having a hearing aid style structure will be very convenient and useful for them. For patients who do not need a hearing aid, an auricular automatic anti-arrhythmia system 200A can be made with a shape adapted from a hearing aid, and can be worn like a hearing aid. Since a hearing aid can be securely attached to the external ear canal 904, tragus 905 and concha 906, 907, area of the external ear 902, this set-up will also serve as a long-term attachment for the automatic anti-arrhythmia systems 200A, 200B, 200C, 460A, 460B, 760A, 760B. (FIGS. 7, 8, 9).

For most patients or wearers 900 with cardiac arrythmia, who wear an automatic anti-arrhythmia system, 200A, 200B, 200C, 460A, 460B, 760A, 760B, having a taVNS unit 30, the taVNS unit 30 will be placed in or around one external ear 902 (preferably the left external ear 902). However, if one-sided (left side) vagus nerve stimulation is not enough to achieve the desired therapeutic effect for the cardiac arrhythmia, the automatic anti-arrhythmia system 200A, 200B, 200C, 460A, 460B, 760A, 760B, may utilize or comprise two taVNS units 30, with one stimulating electrode 31 of a first taVNS unit 30 placed in the first external ear 902 and with one stimulating electrode 31 of a second taVNS unit 30 placed in the second external ear 902. It has been reported that bilateral vagal stimuli could be more effective than unilateral vagal stimuli. If for any reason, the patient cannot tolerate placing ECG electrodes 12, 13, 88, and stimulating electrode 31 of a taVNS unit 31 in or around one external ear 902, the auricular housing structure 11 can be placed in the other external ear 902. Preferably a spare set of automatic anti-arrhythmia system 200A, 200B, 200C, 460A, 460B, 760A, 760B, is available for a wearer 900 with cardiac arrythmia. When the automatic anti-arrhythmia system 200A, 200B, 200C, 460A, 460B, 760A, 760B, is temporarily removed to charge the power source 16, the wearer 900 can wear the spare set.

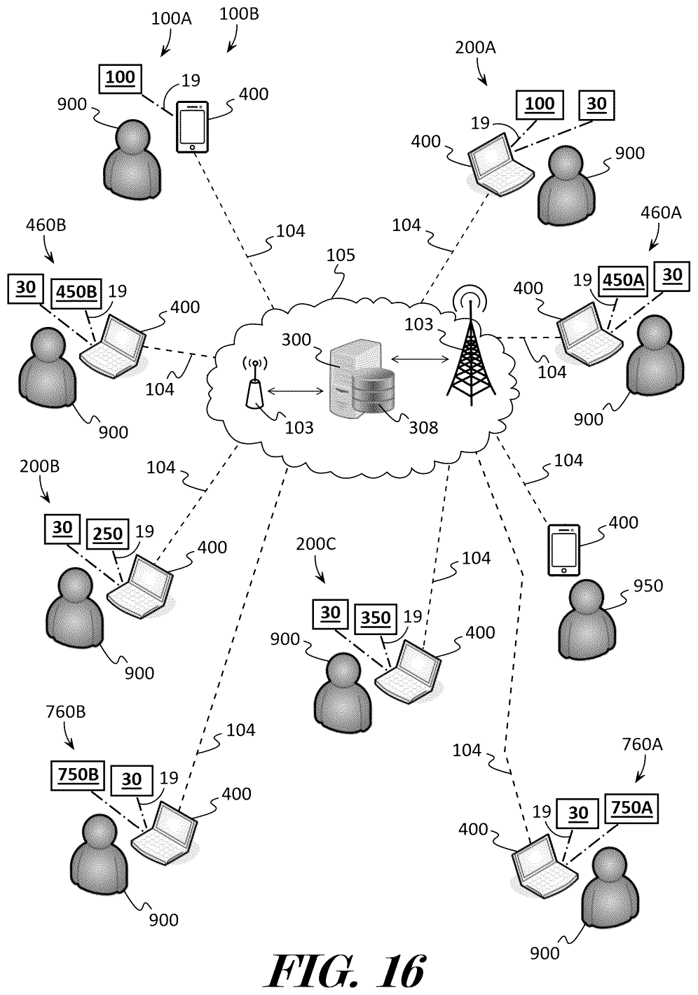

As perhaps best shown by FIG. 16, an illustrative example of some of the physical components which may be used with an ECG monitoring system 100, an auricular automatic anti-arrhythmia system 200A, a chest-based automatic anti-arrhythmia system 200B, an arm-based automatic anti-arrhythmia system 200C, an ear-finger automatic anti-arrhythmia system 460A, a finger-finger automatic anti-arrhythmia system 460B, an ear-toe automatic anti-arrhythmia system 760A and a finger-toe automatic anti-arrhythmia system 760B, according to some embodiments are presented. A system 100, a system 200A, a system 200B, a system 200C, a system 460A, a system 460B, a system 760A and a system 760B configured to facilitate the transfer of data and information between one or more access points 103, client devices 400, and servers 300 over a data network 105. Client devices 400 and servers 300 may send data to and receive data from the data network 105 through a network connection 104 with an access point 103. A data store 308 accessible by the server 300 may contain one or more databases. The data may comprise any data recorded and generated by a system 100, a system 200A, a system 200B, a system 200C, a system 460A and a system 460B.

In this example of FIG. 16, an auricular ECG monitoring system 100, an auricular automatic anti-arrhythmia system 200A, a chest-based automatic anti-arrhythmia system 200B, an arm-based automatic anti-arrhythmia system 200C, an ear-finger automatic anti-arrhythmia system 460A, a finger-finger automatic anti-arrhythmia system 460B, an ear-toe automatic anti-arrhythmia system 760A and a finger-toe automatic anti-arrhythmia system 760B may be in communication with at least one client device 400 (but preferably more than two client devices 400) configured to be operated by one or more users 900, 950. Client devices 400 may include mobile devices, such as laptops, tablet computers, personal digital assistants, smart phones, and the like, that are equipped with a wireless network interface capable of sending data to one or more servers 300 with access to one or more data stores 308 over a network 105, such as a wireless local area network (WLAN). Additionally, client devices 400 may include fixed devices, such as desktops, workstations, and the like, that are equipped with a wireless or wired network interface capable of sending data to one or more servers 300 with access to one or more data stores 308 over a wireless or wired local area network 105. The present invention may be implemented on at least one computing device, such as a client device 400 and/or server 300, programmed to perform the steps described herein. In some embodiments, more than one client device 400 and server 300 may be used, with each being programmed to carry out one or more steps of a process described herein.

### A First Preferred Embodiment

According to one aspect consistent with the principles of the invention, a novel auricular electrocardiogram (ECG) monitoring system 100 is disclosed. An auricular electrocardiogram (ECG) monitoring system 100 that is wearable, convenient, suitable for long-term monitoring and with much less muscle artifacts and movement artifacts is disclosed. The auricular ECG monitoring system 100 may be configured as a single-ear auricular ECG system 100A or a dual-ear auricular ECG system 100B. Referring to FIGS. 4, 5, 6, 10, in some embodiments, a single-ear auricular ECG system 100A may comprise a miniature ECG recording module 20. The ECG recording module 20 may comprise at least two wired or wireless ECG electrodes (ECG sensor electrodes) 12, 13, in electronic communication with the ECG recording module 20. All of the auricular ECG electrodes 12, 13, (for example a first auricular ECG electrode 12 and a second auricular ECG electrode 13) may be configured to be coupled to a wearer's first ear 902 or a peri-auricular area 903 around the wearer's first ear 902. When the first auricular ECG electrode 12 and the second auricular ECG electrode 13 are coupled to the wearer's first ear 902 or the peri-auricular area 903 around the wearer's first ear the first and second auricular ECG electrodes 12, 13, are positioned in contact with the skin of separate locations on an area of the wearer, the area selected from at least one of the following: external ear 902 of the wearer's first ear, external ear canal 904 of the wearer's first ear 902, and the peri-auricular area 903 around the wearer's first ear 902. These locations that the auricular ECG electrodes 12, 13, are attached to may be adequately separated (with different angles or directions) relative to the location of the heart of the wearer so that they can pick up some differences of the cardiac action potentials in order to enable the ECG recording module 20 to perform an ECG. The ECG recording module 20 may be configured to record ECG data of the wearer via the auricular ECG electrodes 12, 13, and the ECG recording module 20 may be in electronic communication with a processing unit 50, 40. With the help of ECG analysis algorithms, the processing unit 50, 401 may be configured to record and analyze the ECG data from the ECG recording module 20 to assess the wearer's ECG profile. The processing unit 50, 401, may be further configured to analyze ECG data recorded by the ECG recording module 20 to detect presence or cessation of a serious cardiac arrhythmia of the wearer 900 and presence or cessation of impending serious cardiac arrhythmia. The serious cardiac arrhythmia may include atrial fibrillation (AFib), atrial flutter (AFL), supraventricular tachycardia (SVT), ventricular tachycardia (VT), and ventricular fibrillation (VF).

As used herein, a peri-auricular area 903 refers to a portion of the head around the auricle (pinna) and this portion of the head 901 is typically hairless. The peri-auricular area 903 includes a portion of the head in front of the auricle (pre-auricular area) and a portion of the head 901 above and behind the auricle (post-auricular area). The pre-auricular area is small, about one inch wide and two inches long and curved along the anterior edge of the auricle. The post-auricular area is also small, about one inch wide and about three inches long and curved along the superior and posterior edges of the auricle (pinna). The post-auricular area is where a behind-the-ear hearing aid is usually located. The small pre-auricular area and post-auricular area together will be called “peri-auricle area” 903 herein. (Anterior, posterior, superior, in front of and behind etc. all refer to the directions relative to the wearer's head 901 when the wearer 900 is in an upright position.) As referred to herein, the external ear canal 904 refers to the part of the ear 902 that connects the visible outer ear (pinna) to the middle ear, essentially the tube that carries sound waves to the eardrum.

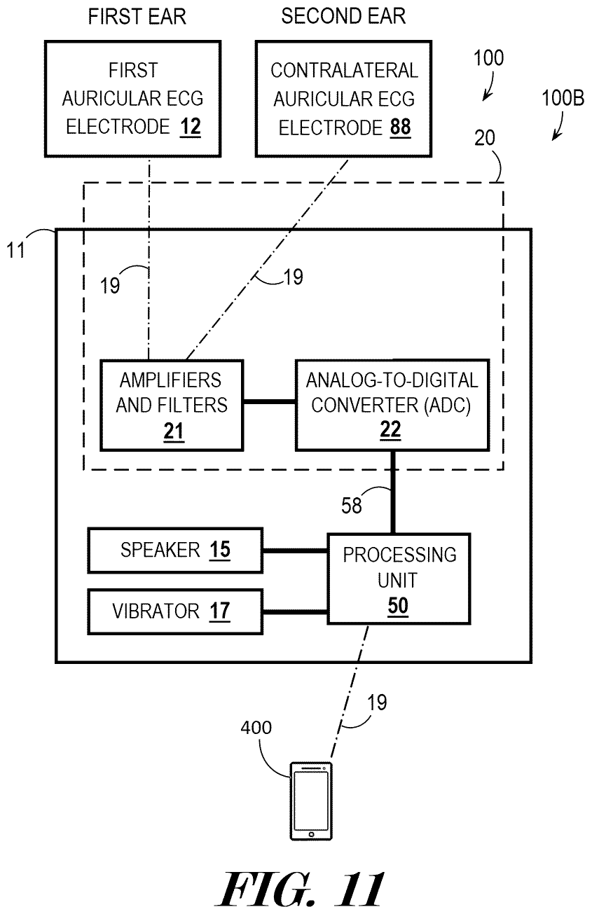

In some embodiments, and referring to FIG. 11, an auricular ECG monitoring system 100 may be configured as a dual-ear auricular ECG system 100B. The dual-ear auricular ECG system 100B may comprise a miniature ECG recording module 20. The ECG recording module 20 may comprise two or more wired or wireless (preferably wireless) auricular ECG electrodes 12, 88, in electronic communication with the ECG recording module 20. At least one of the auricular ECG electrodes (for example a first auricular ECG electrode 12) may be configured to be coupled to a wearer's first ear 902 or a peri-auricular area 903 around the wearer's first ear 902. When the first auricular ECG electrode 12 is coupled to the wearer's first ear 902 or the peri-auricular area 903 around the wearer's first ear the first auricular ECG electrode 12 is positioned in contact with an area of the wearer 900, the area selected from at least one of the following: external ear 902 of the wearer's first ear, external ear canal 904 of the wearer's first ear 902, and the peri-auricular area 903 around the wearer's first ear 902. At least another one of the auricular ECG electrodes 12, 88, (for example a contralateral auricular ECG electrode 88) may be configured to be coupled to the wearer's second ear 902 or a peri-auricular area 903 around the wearer's second ear 902. When the contralateral auricular ECG electrode 88 is coupled to the wearer's second ear 902 or the peri-auricular area 903 around the wearer's second ear 902 the contralateral auricular ECG electrode 88 is positioned in contact with an area of the wearer, the area selected from at least one of the following: external ear 902 of the wearer's second ear, external ear canal 904 of the wearer's second ear 902, and the peri-auricular area 903 around the wearer's second ear 902. In some embodiments, all of the auricular ECG electrodes 12, 88, including the first auricular ECG electrode 12 and the contralateral auricular ECG electrode 88 are configured as wireless ECG electrodes 12, 88, and are in wireless communication with the ECG recording module 20. The ECG recording module 20 may comprise wireless amplifier 21. The ECG recording module 20 may be configured to record ECG data of the wearer via all of the wireless ECG electrodes 12, 88, (including the wireless first auricular ECG electrode 12 and the wireless contralateral auricular ECG electrode 88) and the ECG recording module 20 may be in electronic communication with a processing unit 50, 401. With the help of ECG analysis algorithms, the processing unit 50, 401 may be configured to record and analyze the ECG data from the ECG recording module 20 to assess the wearer's ECG profile. The processing unit 50, 401, may be further configured to analyze ECG data recorded by the ECG recording module 20 to detect presence or cessation of a serious cardiac arrhythmia of the wearer and presence or cessation of an impending serious cardiac arrhythmia of the wearer. The serious cardiac arrhythmia may include atrial fibrillation (AFib), atrial flutter (AFL), supraventricular tachycardia (SVT), ventricular tachycardia (VT), and ventricular fibrillation (VF).

The ECG recording module 20 may be in electronic communication with a processing unit 50, 401, so that the ECG data recorded by the ECG recording module 20 may be communicated to the processing unit 50, 401. With the help of ECG analysis algorithms, the processing unit 50, 401, is configured to analyze the ECG data to detect presence or cessation of any serious cardiac arrhythmia and presence or cessation of any impending serious cardiac arrhythmia, as aforementioned. This auricular ECG monitoring system 100 may further comprise a network interface 53, 406, in electronic communication with the processing unit 50, 401, and at least one of the following: a speaker 15, 404A, a vibrator 17, 404B and a display screen 404C. The network interface 53, 406, may be configured to generate a notification, such as a text message, email, or other electronic user readable message through a display screen 404C to the client device 400 of the wearer 900 of this auricular ECG monitoring system 100 and/or to the client device 400 of the wearer's 900 healthcare provider 950 to take appropriate actions when serious cardiac arrhythmia or impending serious cardiac arrhythmia is detected by the processing unit 50, 401. When cessation of serious cardiac arrhythmia or cessation of impending serious cardiac arrhythmia is detected, the processing unit 50, 401, may be further configured to send a notification to the client device 400 of the wearer 900 of this ECG monitoring system 100 and/or to the client device 400 of the wearer's 900 healthcare provider 950. In some embodiment, the network interface 53, 406, may be configured to send signals to a speaker 15, 404A, to generate an audible notification when serious cardiac arrhythmia or impending serious cardiac arrhythmia is detected. In some embodiment, the network interface 53, 406 may be configured to send signals to a vibrator 17, 404B, to generate a tactile notification when serious cardiac arrhythmia or impending serious cardiac arrhythmia is detected. Additionally, the network interface 53, 406, may be further configured to send signals to the speaker, 15, 404A, vibrator 17, 404B and the display screen 404C to stop the notification when cessation of serious cardiac arrhythmia or cessation of impending serious cardiac arrhythmia is detected.

Referring to FIGS. 1, 4, an example of an earbud style structure 11A of an auricular housing structure 11 of an auricular ECG monitoring system 100 is shown. The first 12 and second 13 auricular ECG electrodes may be located on a surface 25A of a horizontal portion 25 of the earbud (earbud style structure 11A). When the horizontal portion 25 of the earbud-style structure 11A is placed inside an external ear canal 904 and a bowl-shaped area of tragus-concha 905, 906, 907 of a wearer's ear 902, both the first 12 and second 13 auricular ECG electrodes are in close contact with the skin of the external ear canal 904 and skin of the bowl-shaped area of tragus-concha 905, 906, 907. The inside of the horizontal portion of the earbud comprises a sound conduit 18 so that the sound can go through to reach the eardrum. The speaker 15 can provide audible notification when serious cardiac arrhythmia is detected.

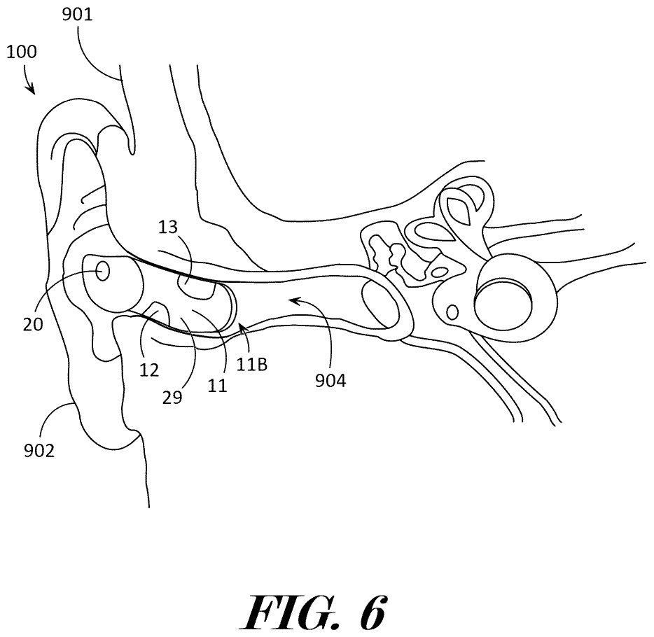

Referring to FIG. 5, another example of an auricular ECG monitoring system 100, housed in a behind-the-ear-hearing-aid-style structure 11C type of auricular housing structure 11 is shown. The behind-the-ear-hearing-aid style structure 11C includes an in-the-ear portion 27 and a behind-the-ear portion 26. The auricular ECG monitoring system 100 is housed in the in-the-ear portion 27 that is configured to be placed into an external ear canal 904 and a bowl-shaped area of tragus-concha 905, 906, 907, of a wearer's ear 902 when in use. There are two auricular ECG electrodes 12, 13, on a surface 27A of the in-the-ear portion 27 of the behind-the-ear-hearing-aid-style structure 11C. Both auricular ECG electrodes 12, 13, contact the skin of external ear canal 904. Referring to FIG. 6, still another example of an auricular ECG monitoring system 100, housed in a tubular-shaped structure 11B type of an auricular housing structure 11 is shown. The auricular ECG monitoring system 100 is inserted into the external ear canal 904. There are two auricular ECG electrodes 12, 13, on the surface 29 of the tubular-shaped structure 11B and both auricular ECG electrodes 12, 13, contact the skin of external ear canal 904.

In some embodiments, auricular housing structures 11 are configured to house all of the auricular ECG electrodes 12, 13, 88, of the auricular ECG monitoring system 100. In some embodiments, a single-ear auricular ECG system 100A of the present invention may comprise an auricular housing structure 11 which may be configured to be coupled to a first ear 902 of a wearer 900 and/or to a peri-auricular area 903 around the first ear 902 of the wearer 900. In some embodiments, a dual-ear auricular ECG system 100B of the present invention may comprise two auricular housing structures 11, with an auricular housing structure 11 may be configured to be coupled to a first ear 902 of a wearer 900 and/or to the peri-auricular area 903 around the first ear 902 of a wearer 900, while another auricular housing structure 11 may be configured to be coupled to a second ear 902 of the wearer 900 and/or to the peri-auricular area 903 around the second ear 902 of the wearer 900.

In some embodiments, each auricular ECG electrode 12, 13, of a single-ear auricular ECG system 100A may be housed in an auricular housing structure 11, such as an earbud-style structure 11A, a tubular-shaped structure 11B, and a behind-the-ear-hearing-aid-style structure 11C. The earbud-style structure 11A may comprise a horizontal portion 25. The horizontal portion 25 is configured to be made from elastic flexible and adaptable material (such as soft foam or ultra-soft silicone-type material). The elastic flexible and adaptable material of the horizontal portion 25 is configured to have appropriate elasticity, flexibility and adaptability such that it will naturally adapt to the contour of the wearer's external ear canal 904 and contour of the bowl-shaped area of tragus-concha 905, 906, 907, and snugly fill the interior of the wearer's external ear canal 904 and interior of the bowl-shaped area of tragus-concha 905, 906, 907, when the horizontal portion is put in place. All of the auricular ECG electrodes 12, 13, may be configured to be partially embedded in a surface 25A of the horizontal portion 25 with slight protrusion at the surface 25A of the horizontal portion 25 such that all of these auricular ECG electrodes 12, 13, will be naturally and snugly in contact with the skin of the wearer's external ear canal 904 or skin of the bowl-shaped area of tragus-concha 905, 906, 907. The behind-the-ear-hearing-aid-style structure 11C may comprise an in-the-ear portion 27 and a behind-the-ear portion 26 and the in-the-ear portion 27 is configured to be put into the wearer's external ear canal 904 and the bowl-shaped area of tragus-concha 905, 906, 907, when in use. The in-the-ear portion 27 is configured to be made from elastic flexible and adaptable material (such as soft foam or ultra-soft silicone-type material). The elastic flexible and adaptable material of the in-the-ear portion 27 is configured to have appropriate elasticity, flexibility and adaptability such that it will naturally adapt to the contour of the wearer's external ear canal 904 and contour of the bowl-shaped area of tragus-concha 905, 906, 907, and snugly fill the interior of the wearer's external ear canal 904 and interior of bowl-shaped area of tragus-concha 905, 906, 907, when the in-the-ear portion 27 is placed into the wearer's external ear 902. Each auricular ECG electrode 12, 13, may be configured to be partially embedded in a surface 27A of the in-the-ear portion 27 with slight protrusion at the surface 27A of the in-the-ear portion 27 such that all of these auricular ECG electrodes 12, 13, will be naturally and snugly in close contact with the skin of the wearer's external ear canal 904 or skin of the bowl-shaped area of tragus-concha 905, 906, 907. Optionally, all of the auricular ECG electrodes 12, 13, of the single-ear auricular ECG system 100A may be housed in a tubular-shaped structure 11B. The tubular-shaped structure 11B is configured to be made from elastic flexible and adaptable material (such as soft foam or ultra-soft silicone-type material). The elastic flexible and adaptable material of the tubular-shaped structure 11B is configured to have appropriate elasticity, flexibility and adaptability such that it will naturally adapt to the contour of the wearer's external ear canal 904 and snugly fill the interior of the wearer's external ear canal 904 when the tubular-shaped structure 11B is inserted into the wearer's external ear canal 904. All of the auricular ECG electrodes 12, 13, may be configured to be partially embedded in a surface 29 of the tubular-shaped structure 11B with slight protrusion at the surface 29 of the tubular-shaped structure 11B such that all of these auricular ECG electrodes 12, 13, will be naturally and snugly in close contact with the skin of the wearer's external ear canal 904. Installing and removing these auricular ECG electrodes 12, 13, will be as easy as inserting and removing the earbud-style structure 11A, the behind-the-ear-hearing-aid-style structure 11C or the tubular-shaped structure 11B from the wearer's external ear 902. This will allow easy self-installation and easy self-removal of the auricular ECG electrodes 12, 13, by the wearer (user) 900.

In some embodiments, a dual-ear auricular ECG system 100B of the present invention may comprise two auricular housing structures 11, including a first auricular housing structure 11 and a second auricular housing structure 11. Optionally, the first auricular housing structure 11 is configured to be coupled to a first ear 902 of a wearer 900 and/or to the peri-auricular area 903 around the first ear 902 of a wearer 900, while the second auricular housing structure 11 may be configured to be coupled to a second ear 902 of the wearer 900 and/or to the peri-auricular area 903 around the second ear 902 of the wearer 900. At least one of the auricular ECG electrodes (for example a first auricular ECG electrode 12) of a dual-ear auricular ECG system 100B may be housed in a first in-the-ear portion 27 of the first auricular housing structure 11 and the first in-the-ear portion 27 is configured to be put into an external ear canal 904 and a bowl-shaped area of tragus-concha 905, 906, 907, of the wearer's first ear 902 when in use. At least another auricular ECG electrode (for example a contralateral auricular ECG electrode 88) may be configured to be housed in a second in-the-ear portion 27 of the second auricular housing structure 11 and the second in-the-ear portion 27 is configured to be put into an external ear canal 904 and a bowl-shaped area of tragus-concha 905, 906, 907, of the wearer's second ear 902 when in use. The first auricular housing structure 11 may be selected from one of the following: an earbud-style structure 11A, a tubular-shaped structure 11B, and a behind-the-ear-hearing-aid-style structure 11C. The first in-the-ear portion 27 of the first auricular housing structure 11 (including 11A, 11B, 11C) may be configured to be made of flexible, elastic and adaptable material (such as soft form or soft silicone-type material) and the first auricular ECG electrode 12 is configured to be partially embedded in a surface 27A of the first in-the-ear portion 27 of the first auricular housing structure 11 with slight protrusion at the surface 27A of the first in-the-ear portion 27, similar to descriptions hereinbefore. Installing and removing the first ECG electrode 12 will be as easy as inserting and removing the first auricular housing structure 11 (including the earbud-style structure 11A, the behind-the-ear-hearing-aid-style structure 11C or the tubular-shaped structure 11B) from the wearer's first external ear 902. These features will enable the first ECG electrode 12 to be easily self-installable and easily self-removable.

Similar housing setups may be applied to the contralateral ECG electrode 88 that may be configured to be housed in the second in-the-ear portion 27 of the second auricular housing structure 11 and the second in-the-ear portion 27 is configured to be put into the external ear canal 904 and the bowl-shaped area of tragus-concha 905, 906, 907 of the wearer's second ear 902 when in use. The second auricular housing structure 11 may be selected from one of the following: a second earbud-style structure 11A, a second tubular-shaped structure 11B, and a second behind-the-ear-hearing-aid-style structure 11C. (It should be noted that the housing structure selected for the first auricular housing structure 11 may be the same or may be different from the housing structure selected for the second auricular housing structure 11.) The second auricular housing structures 11 (including 11A, 11B, 11C) may be configured to be made of flexible, elastic and adaptable material (such as soft form or soft silicone-type material) and the contralateral auricular ECG electrode 88 is configured to be partially embedded in a surface 27A of the second in-the-ear portion 27 of the second auricular housing structure 11 with slight protrusion at the surface 27A of the second in-the-ear portion 27, similar to descriptions hereinbefore. Installing and removing the contralateral auricular ECG electrode 88 will be as easy as inserting and removing the second in-the-ear portion 27 of the second auricular housing structure 11 (including the earbud-style structure 11A, the behind-the-ear-hearing-aid-style structure 11C or the tubular-shaped structure 11B) from the wearer's second external ear 902. These features will enable the contralateral ECG electrode 88 to be easily self-installable and easily self-removable. There is no need to have a technologist to pre-prepare the skin surface before attaching the electrodes and also no need for the technologist to use special adhesive material to secure the electrodes to the user's skin. These features and setups will also enable the in-the-ear portion 27 of the auricular housing structure 11 (including the earbud-style structure 11A, the tubular-shaped structure 11B and the in-the-ear portion 27 of the behind-the-ear-hearing-aid-style structure 11C) of this invention to be collectively referred as “self-installable-housing structures”.

### A Second Preferred Embodiment

According to another aspect consistent with the principles of the invention, an automatic anti-arrhythmia system that may be configured as an auricular automatic anti-arrhythmia system 200A is disclosed which may comprise a novel integration of the aforementioned auricular ECG monitoring system 100 (including system 100A and system 100B) and a transcutaneous auricular vagus nerve stimulation (taVNS) unit 30, together with a processing unit 50, 401. Details of the auricular ECG monitoring system 100, including the single-ear auricular ECG system 100A and the dual-ear auricular ECG system 100B have been described above and in the First Preferred Embodiment.

The auricular automatic anti-arrhythmia system 200A may further comprise a transcutaneous auricular vagus nerve stimulation (taVNS) unit 30. The taVNS unit 30 comprises a miniature stimulating electrode 31 that is configured to contact or be attached to vagus nerve innervated auricular skin of the wearer 900. The vagus innervated auricular skin includes external ear canal 904, tragus 905, cymba-concha 906, cavum-concha 907 and small adjacent areas. The vagus innervated auricular skin that the stimulating electrode 31 is configured to contact may be selected from at least one of the following: external ear canal 904, tragus 905, cymba-concha 906, and cavum-concha 907 of the wearer's ear 902. The ECG recording module 20 may be in wired or wireless electronic communication (e.g., through wire, Bluetooth, etc.) with a processing unit 50, 401. The taVNS unit 30 may also be in electronic communication with the processing unit 50, 401. The processing unit 50, 401, may be configured to analyze ECG data recorded by the auricular ECG monitoring system 100 (including system 100A or system 100B) to detect the presence or cessation of ECG signals suggestive of serious cardiac arrhythmias or impending serious cardiac arrhythmias. When the presence of ECG signals suggestive of at least one serious cardia arrhythmia is detected by the processing unit 50, 401, the processing unit 50, 401, may be configured to immediately send arrhythmia-warning-signals to the taVNS unit 30 and upon receiving the arrhythmia-warning-signals from the processing unit 50, 401, the taVNS unit 30 is configured to automatically start sending pre-determined electric stimuli to the vagus innervated auricular skin of the wearer's ear 902 to which the stimulating electrode 31 is in contact with (examples of stimulating parameters as in Table 1). When the presence of ECG signals suggestive of at least one impending serious cardia arrhythmia is detected by the processing unit 50, 401, the processing unit 50, 401, may be configured to immediately send impending-arrhythmia-warning-signals to the taVNS unit 30 and upon receiving the impending-arrhythmia-warning-signals from the processing unit 50, 401, the taVNS unit 30 is configured to automatically start sending pre-determined electric stimuli to the vagus innervated auricular skin of the wearer's ear 902 to which the stimulating electrode 31 is in contact with (examples of stimulating parameters as in Table 2). When cessation of ECG signals suggestive of the at least one serious cardiac arrhythmias is detected by the processing unit 50, 401, the processing unit 50, 401, may be further configured to immediately send arrhythmia-cessation-signals to the taVNS unit 30 and upon receiving arrhythmia-cessation-signals from the processing unit the taVNS unit 30 is configured to automatically stop sending pre-determined electric stimuli to the vagus innervated auricular skin of the wearer's ear 902 to which the stimulating electrode 31 is in contact with. When cessation of ECG signals suggestive of the at least one impending serious cardiac arrhythmias is detected by the processing unit 50, 401, the processing unit 50, 401, may be further configured to immediately send impending-arrhythmia-cessation signals to the taVNS unit 30 and upon receiving the impending-arrhythmia-cessation signals from the processing unit the taVNS unit 30 is configured to automatically stop sending pre-determined electric stimuli to the vagus innervated auricular skin of the wearer's ear 902 to which the stimulating electrode 31 is in contact with.

In some embodiments, an auricular automatic anti-arrhythmia system 200A may comprise one or two transcutaneous auricular vagus nerve stimulation (taVNS) units 30 (“taVNS unit 30”) such as shown in FIG. 21. Optionally, an auricular automatic anti-arrhythmia system 200A may comprise a first taVNS unit 30 and a second taVNS unit 30. Preferably, the first taVNS unit 30 comprises a first stimulating electrode 31 that is configured to contact vagus innervated auricular skin of the wearer's first ear 902, while the second taVNS unit 30 comprises a second stimulating electrode 31 that is configured to contact vagus innervated auricular skin of the wearer's second ear 902. (It had been reported that bilateral vagus nerve stimulation with bilateral taVNS units might be more effective than unilateral vagus nerve stimulation). A processing unit 50, 401, may be in electronic communication with the first and the second taVNS units 30. When the presence of ECG signals suggestive of a serious cardiac arrhythmia is detected by the processing unit 50, 401, the processing unit 50, 401, may be configured to immediately send arrhythmia-warning-signals to the first and the second taVNS units 30 to cause the first and the second ta VNS units 30 to automatically start sending pre-determined electric stimuli to the vagus innervated auricular skin of the wearer's first and second ears 902 (examples of stimulation parameters as in Table 1). When the presence of ECG signals suggestive of an impending serious cardiac arrhythmia is detected by the processing unit 50, 401, the processing unit 50, 401, may be configured to immediately send impending-arrhythmia-warning-signals to the first and the second taVNS units 30 to cause the first and the second taVNS units 30 to automatically start sending pre-determined electric stimuli to the vagus innervated auricular skin of the wearer's first and second ears 902. (examples of stimulation parameters as shown in Table 2). When cessation of ECG signals suggestive of the serious cardiac arrhythmia or the impending cardiac arrhythmia is detected by the processing unit 50, 401, the processing unit 50, 401, may be further configured to immediately send arrhythmia-cessation-signals or impending-arrhythmia-cessation signals to the first and the second taVNS units 30 to cause the first and the second taVNS units 30 to automatically stop or modify sending pre-determined electric stimuli to the vagus innervated auricular skin of the wearer's first and second ears 902.

A taVNS unit 30 may comprise any device that is able to provide transcutaneous auricular vagus nerve stimulation to a user's body. As an example, and referring to FIG. 13, a taVNS unit 30 may comprise a microcontroller 33 that may be in communication with a pulse generator 34, voltage regulator 35, voltage transformer 36, amplifier 37, and buffer 38, and that may be configured to generate taVNS stimuli that may be delivered to vagus nerve innervated auricular skin via a stimulating electrode 31. The taVNS unit 30 comprises a stimulating electrode 31 which can give pre-determined electric current transcutaneously in a way similar to transcutaneous electrical nerve stimulation (TENS) (taVNS and TENS as known in the art). The stimulus parameters of the electric stimuli (including strength, pattern, duration, interval, frequency and timing etc.) are pre-determined, such as examples disclosed in Tables 1 & 2. The taVNS unit 30 contains a stimulating electrode 31 for delivering of electric stimuli to the vagus innervated auricular skin (including tragus 905, concha 906, 907, or external ear canal 904) that the stimulating electrode 31 is attached to so that the electric stimuli can be delivered to the auricular branch of vagus nerve.

The ECG recording module 20 and the taVNS unit 30 may both be in electronic communication 19 with a processing unit 50, 401, through Bluetooth, a wired connection, or other electronic connecting means. The ECG data recorded by the ECG recording module 20 are transmitted or otherwise communicated to the processing unit 50, 401. The processing unit 50, 401, may be configured to analyze the ECG data to detect presence or cessation of any serious cardiac arrhythmia and presence or cessation of any impending serious cardiac arrhythmia, including AFib, AFL, SVT, VT, VF or other cardiac arrhythmia, using ECG analysis algorithms (ECG analysis algorithms as known in the art). Upon detection of presence of a serious cardiac arrhythmia, the processing unit 50, 401, may be configured to automatically send arrhythmia-warning-signals to actuate the taVNS unit 30 to immediately start sending pre-determined electric stimuli to the auricular branch of vagus nerve via the stimulating electrode 31 (examples of stimulating parameters as shown in Table 1). Upon detection of presence of an impending serious cardiac arrhythmia, the processing unit 50, 401, may be configured to automatically send impending-arrhythmia-warning-signals to actuate the ta VNS unit 30 to immediately start sending pre-determined electric stimuli to the auricular branch of vagus nerve via the stimulating electrode 31 (examples of stimulating parameters as shown in Table 2). When the processing unit 50, 401, detects cessation of the serious cardiac arrhythmia or cessation of the impending serious cardiac arrhythmia, the processing unit 50, 401, may be configured to send arrhythmia-cessation-signals or impending-arrhythmia-cessation signals to the taVNS unit 30 to modify or stop the electric stimuli to the vagus nerve. This auricular automatic anti-arrhythmia system 200A may automatically initiate therapeutic intervention instantly when a serious cardiac arrhythmia or an impending serious cardiac arrhythmia is detected. This can avoid the inevitable delay associated with the traditional set-up where the ECG data being sent to a monitoring center and the monitoring center notifying healthcare provider 950 via their client device 400 to arrange for therapeutic intervention.

In some embodiments, an automatic anti-arrhythmia system 200A comprises an auricular ECG monitoring system 100, a transcutaneous auricular vagus nerve stimulation (taVNS) unit 30 and a processing unit 50, 401. The auricular ECG monitoring system 100 and the taVNS unit 30 may be housed together or separately. In some embodiments, an auricular automatic anti-arrhythmia system 200A that comprises a single-ear auricular ECG system 100A may have a first auricular housing structure 11. The first auricular housing structure 11 includes a first in-the-ear portion 27 that is configured to be put into an external ear canal 904 and a bowl-shaped area of tragus-concha 905, 906, 907, of a wearer's first ear 902 when in use. In some embodiments an automatic anti-arrhythmia system 200A with a single-ear auricular ECG system 100A may be optionally configured to have a first auricular housing structure 11 that is configured to house all of the auricular ECG electrodes 12, 13, of the auricular ECG monitoring system 100 and the stimulating electrode 31 of the taVNS unit 30. The auricular ECG electrodes 12, 13, of a single-ear auricular ECG system 100A and the stimulating electrode 31 of the taVNS unit 31 may be housed in a first in-the-ear portion 27 of a first auricular housing structure 11. The auricular ECG electrodes 12, 13 and the stimulating electrode 31 are configured to be located at separate locations on a surface 27A of the first in-the-ear portion 27 of the first auricular housing structure 11. The first auricular housing structure 11 may be selected from one of the following: a first earbud-style structure 11A, a first tubular-shaped structure 11B, and a first behind-the-ear-hearing-aid-style structure 11C. Optionally, in some embodiments, a first earbud-style structure 11A may be used for housing purpose. The first earbud-style structure 11A may comprise a first horizontal portion 25 (a horizontal portion 25 of an earbud-style structure 11A is equivalent to an in-the-ear portion 27 of an auricular housing structure 11). The first horizontal portion 25 of the first earbud-style structure 11A is configured to be made from elastic flexible and adaptable material (such as soft foam or ultra-soft silicone-type material). The elastic flexible and adaptable material of the first horizontal portion 25 is configured to have appropriate elasticity, flexibility and adaptability such that it will naturally adapt to the contour the external ear canal 904 and the contour of a bowl-shaped area of tragus-concha 905, 906, 907, of the wearer's first ear 902 (a bowl-shaped area of tragus-concha 905, 906, 907 is essentially the same as an area surrounding the opening of an external ear canal 904), and snugly fill the interior of the external ear canal 904 and the bowl-shaped area of tragus-concha 905, 906, 907, of the wearer's first ear 902 when the first horizontal portion 25 is put into the wearer's first ear 902 (or first external ear 902). All of the auricular ECG electrodes 12, 13, and the stimulating electrode 31 may be configured to be partially embedded in a surface 25A of the first horizontal portion 25 with slight protrusion at the surface 25A of the first horizontal portion 25 such that all of these electrodes 12, 13, 31, will be naturally and snugly in contact with the skin of the external ear canal 904 and skin of bowl-shaped area of tragus-concha 905, 906, 907, of the wearer's first ear 902. Optionally, in some embodiments, a first behind-the-ear-hearing-aid-style structure 11C may be used for housing purpose. The first behind-the-ear-hearing-aid-style structure 11C may comprise a first in-the-ear portion 27 (an in-the-ear portion 27 of a behind-the-ear-hearing-aid-style structure 11C is essentially the same as an in-the-ear portion 27 of an auricular housing structure 11). The first in-the-ear portion 27 is configured to be made from elastic flexible and adaptable material (such as soft foam or ultra-soft silicone-type material). The elastic flexible and adaptable material of the first in-the-ear portion 27 is configured to have appropriate elasticity, flexibility and adaptability such that it will naturally adapt to the contour of the wearer's external ear canal 904 and the bowl-shaped area of tragus-concha 905, 906, 907, of the wearer's first ear 902 and snugly fill the interior of the external ear canal 904 and interior of the bowl-shaped area of tragus-concha 905, 906, 907, of the wearer's first ear 902 when the first in-the-ear portion 27 is placed into the wearer's first ear 902. All of the auricular ECG electrodes 12, 13, and the stimulating electrode 31 may be configured to be partially embedded in a surface 27A of the first in-the-ear portion 27 with slight protrusion at the surface 27A of the first in-the-ear portion 27 such that all of these electrodes 12, 13, 31, will be naturally and snugly in contact with the skin of the external ear canal 904 and skin of the bowl-shaped area of tragus-concha 905, 906, 907, of the wearer's first ear 902. Optionally, in some other embodiments, a tubular-shaped structure 11B may be used for housing purpose. The auricular ECG electrodes 12, 13, of the single-ear auricular ECG system 100A and the stimulating electrode 31 of the taVNS unit 30 may be housed in a first tubular-shaped structure 11B (a tubular-shaped structure 11B is part of an in-the-ear portion 27 of an auricular housing structure 11). The first tubular-shaped structure 11B is configured to be made from elastic flexible and adaptable material (such as soft foam or ultra-soft silicone-type material). The elastic flexible and adaptable material of the first tubular-shaped structure 11B is configured to have appropriate elasticity, flexibility and adaptability such that it will naturally adapt to the contour of the wearer's first external ear canal 904 and snugly fill the interior of the wearer's first external ear canal 904 when the first tubular-shaped structure 11B is inserted into the wearer's first external ear canal 904. All of the auricular ECG electrodes 12, 13, and the stimulating electrode 31 may be configured to be partially embedded in a surface 29 of the first tubular-shaped structure 11B with slight protrusion at the surface 29 of the first tubular-shaped structure 11B such that all of these electrodes 12, 13, 31, will be naturally and snugly in close contact with the skin of the external ear canal 904 of the wearer's first ear. Installing and removing these electrodes 12, 13, 31, will be as easy as inserting and removing the first earbud-style structure 11A, the first behind-the-ear-hearing-aid-style structure 11C or the first tubular-shaped structure 11B from the wearer's first ear 902. These features will enable these electrodes 12, 13, 31, to be easily self-installable and easily self-removable.

In some embodiments, an auricular automatic anti-arrhythmia system 200A with a dual-ear auricular ECG system 100B may have the at least two auricular ECG electrodes 12, 88 (including a first auricular ECG electrode 12 and a contralateral auricular ECG electrode 88) and a stimulating electrode 31. The stimulating electrode 31 may be housed together or housed separately with one of the auricular ECG electrodes 12, 88. Optionally, the stimulating electrode 31 may be housed together with the first auricular ECG electrode 12 in a first in-the-ear portion 27 of a first auricular housing structure 11. Alternatively, the stimulating electrode 31 may be housed together with the contralateral auricular ECG electrode 88 in a second in-the-ear portion 27 of a second auricular housing structure 11.

In some embodiments, an auricular automatic anti-arrhythmia system 200A that comprises a dual-ear auricular ECG system 100B may preferably have two auricular housing structures 11, including a first and a second auricular housing structures 11. The first auricular housing structure 11 may include a first in-the-ear portion 27 that is configured to be put into an external ear canal 904 and a bowl-shaped area of tragus-concha 905, 906, 907, of a first ear 902 of the wearer when in use. The second auricular housing structure 11 may include a second in-the-ear portion 27 that is configured to be put into an external ear canal 904 and a bowl-shaped area of tragus-concha 905, 906, 907, of the wearer's second ear 902 when in use. For an auricular automatic anti-arrhythmia system 200A having a dual-ear auricular ECG system 100B, the first auricular ECG electrode 12 may be housed in the first in-the-ear portion 27 of the first auricular housing structure 11. The first auricular housing structure 11 may be configured in a similar way as aforementioned descriptions. Optionally, the contralateral auricular ECG electrode 88 and the stimulating electrode 31 of the taVNS unit 30 may be housed together in the second in-the-ear portion 27 of the second auricular housing structure 11. The second in-the-ear portion 27 is configured to be placed into the second external ear canal 904 and the second bowl-shaped area of tragus-concha 905, 906, 907, of a wearer's second ear 902 when in use. The second auricular housing structure 11 may be selected from one of the following: a second earbud-style structure 11A, a second behind-the-ear-hearing-aid-style structure 11C and a second tubular-shaped structure 11B. Optionally, in some embodiments, a second earbud-style structure 11A may be used for housing purpose. The second earbud-style structure 11A may comprise a second horizontal portion 25 (a horizontal portion of an earbud-style structure is equivalent to an in-the-ear portion 27 of an auricular housing structure 11). The contralateral auricular ECG electrode 88 and the stimulating electrode 31 of the taVNS unit 30 may be configured to be partially embedded in a surface 25A of the second horizontal portion 25 of the second earbud-style structure 11A with slight protrusion at the surface 25A of the second horizontal portion 25 of the second earbud-style structure 11A. The second horizontal portion 25 may comprise a flexible elastic and adaptable material (e.g. soft foam or very soft silicone-type material) and the flexible elastic and adaptable material is configured to have appropriate flexibility, elasticity and adaptability such that the second horizontal portion 25 will naturally adapt to the contour of the external ear canal 904 and contour of the bowl-shaped area of tragus-concha 905, 906, 907, of the wearer's second ear 902 and snugly fill the interior of the external ear canal 904 and the bowl-shaped area of tragus-concha 905, 906, 907, of the wearer's second ear 902 and such that the contralateral auricular ECG electrode 88 and the stimulating electrode 31 are naturally and snugly in close contact with the skin of the external ear canal 904 and skin of the bowl-shaped area of tragus-concha 905, 906, 907, of the wearer's second ear 902 when the second horizontal portion 25 of the second earbud-style structure 11A is placed into the wearer's second ear 902. Optionally, in some embodiments, a second behind-the-ear-hearing-aid-style structure 11C may be used for housing purpose, The second behind-the-ear-hearing-aid-style structure 11C may comprise a second in-the-ear portion 27 (an in-the-ear portion 27 of a behind-the-ear-hearing-aid-style structure 11C is essentially the same as an in-the-ear portion 27 of an auricular housing structure 11). The contralateral auricular ECG electrode 88 and the stimulating electrode 31 of the taVNS unit 30 may be configured to be partially embedded in a surface 27A of the second in-the-ear portion 27 with slight protrusion at the surface 27A of the second in-the-ear portion 27. The second in-the-ear portion 27 may comprise a flexible elastic and adaptable material (such as soft foam or very soft silicone-type material) and the flexible elastic and adaptable material is configured to have appropriate flexibility, elasticity and adaptability such that the second in-the-ear portion 27 will naturally adapt to the contour of the external ear canal 904 and contour of the bowl-shaped area of tragus-concha 905, 906, 907, of the wearer's second ear 902 and snugly fill the interior of the external ear canal 904 and bowl-shaped area of tragus-concha 905, 906, 907, of the wearer's second ear 902 and such that the contralateral auricular ECG electrode 88 and the stimulating electrode 31 are naturally and snugly in close contact with the skin of the external ear canal 904 and skin of the bowl-shaped area of tragus-concha 905, 906, 907, of the wearer's second ear 902 when the second in-the-ear portion 27 is put in place. Optionally, in some other embodiments, a second tubular-shaped structure 11B may be used for housing purpose. The contralateral auricular ECG electrode 88 of the dual-ear auricular ECG system 100A and the stimulating electrode 31 of the taVNS unit 30 may be housed in the second tubular-shaped structure 11B (a tubular-shaped structure 11B is essentially part of an in-the-ear portion 27 of an auricular housing structure 11). The contralateral auricular ECG electrode 88 and the stimulating electrode 31 of the taVNS unit 30 may be configured to be partially embedded in a surface 29 of the second tubular-shaped structure 11B with slight protrusion at the surface 29 of the second tubular-shaped structure 11B. The second tubular-shaped structure 11B is configured to be made from elastic flexible and adaptable material (such as soft foam or ultra-soft silicone-type material). The elastic flexible and adaptable material of the second tubular-shaped structure 11B is configured to have appropriate elasticity, flexibility and adaptability such that the second tubular-shaped structure 11B will naturally adapt to the contour of the wearer's second external ear canal 904 and snugly fill the interior of the wearer's second external ear canal 904 when the tubular-shaped structure 11B is inserted into the wearer's second external ear canal 904 and such that the contralateral auricular ECG electrode 88 and the stimulating electrode 31 are naturally and snugly in close contact with the skin of the wearer's second external ear canal 904 when the tubular-shaped structure 11B is inserted into the wearer's second external ear canal 904. Installing and removing the contralateral auricular ECG electrode 88 and the stimulating electrode 31 will be as easy as inserting and removing the second earbud-style structure 11A, the second behind-the-ear-hearing-aid-style structure 11C or the second tubular-shaped structure 11B from the wearer's second external ear 902. These features will enable the contralateral auricular ECG electrode 88 and the stimulating electrode 31 of the taVNS unit to be easily self-installable and self-removable.

In some embodiments, an auricular automatic anti-arrhythmia system 200A having a dual-ear auricular ECG system 100B may optionally comprises two taVNS units with a first taVNS unit 30 having a first stimulating electrode 31 coupled to a first ear 902 of a wearer 900 and a second taVNS unit having a second stimulating electrode 31 coupled to a second ear 902 of the wearer 900. Optionally, the first auricular ECG electrode 12 and the first stimulating electrode 31 may be housed together. Optionally, the contralateral auricular ECG electrode 88 and the second stimulating electrode 31 may be housed together. The first auricular ECG electrode 12 and the first stimulating electrode 31 of the first taVNS unit 30 may be housed together in a first in-the-ear portion 27 of a first auricular housing structure 11 and the first in-the-ear portion 27 is configured to be put into a first external ear canal 904 and first bowl-shaped area of tragus-concha 905, 906, 907, of the wearer's first ear 902 when in use. The first auricular housing structure 11 may be selected from one of the following: an earbud-style structure 11A, a tubular-shaped structure 11B, and a behind-the-ear-hearing-aid-style structure 11C. The first in-the-ear portion 27 of the first auricular housing structure 11 (including 11A, 11B, 11C) may be configured to be made of flexible, elastic and adaptable material (such as soft form or soft silicone-type material) and the first auricular ECG electrode 12 and the first stimulating electrode 31 of the first taVNS unit 30 are configured to be partially embedded in a surface 27A of the first in-the-ear portion 27 of the first auricular housing structure 11 with slight protrusion at the surface 27A of the first in-the-ear portion 27, similar to descriptions hereinbefore. Likewise, the contralateral auricular ECG electrode 88 and the second stimulating electrode 31 of the second taVNS unit 30 may be housed together in a second in-the-ear portion 27 of a second auricular housing structure 11 and the second in-the-ear portion 27 is configured to be placed into a second external ear canal 904 and second bowl-shaped area of tragus-concha 905, 906, 907, of the wearer's second ear 902 when in use. The second auricular housing structure 11 may be selected from one of the following: an earbud-style structure 11A, a tubular-shaped structure 11B, and a behind-the-ear-hearing-aid-style structure 11C. The second in-the-ear portion 27 of the second auricular housing structure 11 (including 11A, 11B, 11C) may be configured to be made of flexible, elastic and adaptable material (such as soft form or soft silicone-type material) and the contralateral auricular ECG electrode 88 and the second stimulating electrode 31 of the second taVNS unit 30 are configured to be partially embedded in a surface 27A of the second in-the-ear portion 27 of the second auricular housing structure 11 with slight protrusion at the surface 27A of the second in-the-ear portion 27, similar to descriptions hereinbefore. Similar descriptions are applicable when an earbud-style structure 11A, a behind-the-ear-hearing-aid-style structure 11C or a tubular-shaped structure 11B is used for housing purpose. These setups will enable the electrodes 12, 88, 31 to be naturally and snugly in close contact with the skin of external ear canal 904 or skin of bowl-shaped area of tragus-concha 905, 906, 907, of the wearer's first and second ears 902 when these auricular housing structures 11 are put in use. Installing and removing the first auricular ECG electrode 12, the contralateral ECG electrode 88 and the stimulating electrodes 31 will be as easy as placing and removing the first and second auricular housing structures 11 (including the earbud-style structure 11A, the behind-the-ear-hearing-aid-style structure 11C or the tubular-shaped structure 11B) from the wearer's first and second ears 902. These features will allow easy self-installation and easy self-removal of the auricular ECG electrodes 12, 88, and the stimulating electrodes 31 of the taVNS units 30. The stimulating electrode 31 will naturally in close contact with vagus innervated auricular skin since external ear canal 904, tragus 905 and concha 906, 907, all have rich vagal innervation.

In some embodiments, an auricular automatic anti-arrhythmia system 200A may comprise a network interface 53, 406, and the network interface 53, 406, may be configured to generate an electronic notification via an input/output (I/O) interface 404 of a client device 400 when the processing unit 50, 401, detects the presence or cessation of a serious cardiac arrhythmia or an impending serious cardiac arrhythmia. In preferred embodiments, the auricular automatic anti-arrhythmia system 200A may comprise one or more client devices 400 that may be configured to generate a notification when a processing unit 50, 401, detects the presence or cessation of a serious cardiac arrhythmia or impending serious cardiac arrhythmia. The auricular automatic anti-arrhythmia system 200A preferably further includes notification function, such as through a speaker 15, 404A and/or a vibrator 17, 404B. The speaker 15, 404A may be configured to make audible notification, such as a voice, tone, beeping noise, or other audible alert about presence of a serious cardiac arrhythmia (including atrial fibrillation, atrial flutter, supraventricular tachycardia, ventricular tachycardia, or ventricular fibrillation) is detected by a processing unit 50, 401. Similarly, the speaker 15, 404A may be configured to make audible notification when the processing unit 50, 401, detects presence of an impending serious cardiac arrhythmia. The vibrator 17, 404B can make tactile notification when the processing unit 50, 401 detects presence of serious cardiac arrhythmia or impending serious cardiac arrhythmia.

### A Third Preferred Embodiment

According to another aspect consistent with the principles of the invention, an automatic anti-arrhythmia system may be configured as a chest-based automatic anti-arrhythmia system 200B or an arm-based automatic anti-arrhythmia system 200C.

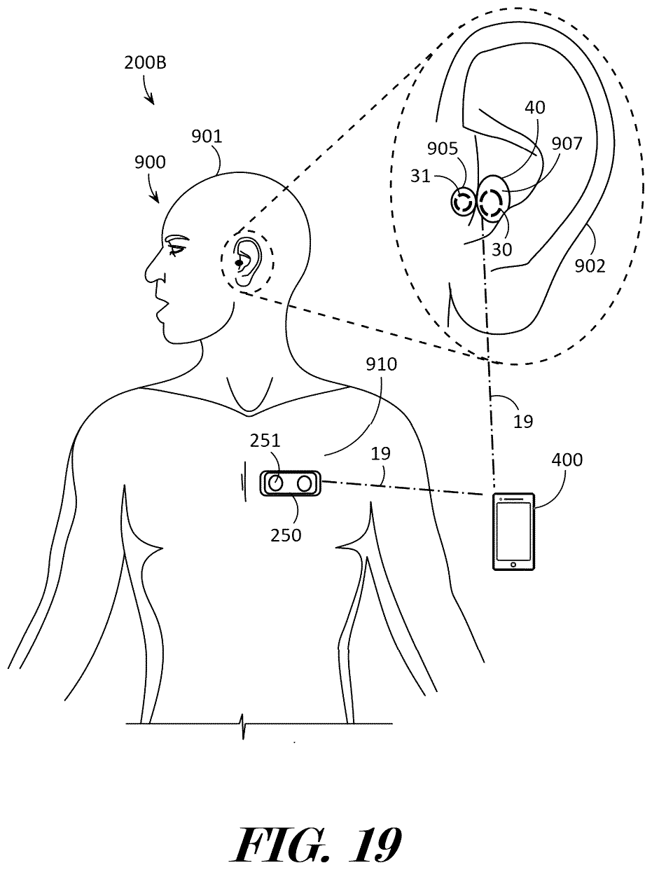

In some embodiments, and referring to FIGS. 14 and 19, a chest-based automatic anti-arrhythmia system 200B may comprise a chest-based ECG monitoring system 250, a transcutaneous auricular vagus nerve stimulation (taVNS) unit 30 and a processing unit 50, 401. The chest-based ECG monitoring system 250 comprises a chest-based ECG recording module 256 that is in electronic communication with at least two chest-contacting electrodes 251. The chest-based ECG recording module 256 is configured to record ECG data via the at least two chest-contacting electrodes 251. The processing unit 50, 401 is in electronic communication with the chest-based ECG monitoring system 250 and the taVNS unit 30. Examples of a chest-based ECG monitoring system 250 may include a Holter monitor, a Zio Patch Monitor or an Assert-IQ ICM from Abbott. The taVNS unit 30 may be housed in a taVNS housing 40 that preferably may be integrated with an in-the-ear portion 27 of an auricular housing structure 11 and the in-the-ear portion 27 is configured to be placed inside an external ear canal 904 and a bowl-shaped area of concha-inner tragus 907, 906, 905, of a wearer's ear 902 when in use. Alternatively, the taVNS housing 40 may be attached to a location on an external ear 902 or a peri-auricular area 903. The taVNS unit 30 may comprise a stimulating electrode 31 which is configured to give pre-determined electric stimuli transcutaneously to a vagus innervated auricular skin (for example, one of the following: external ear canal 904, tragus 905, cymba concha 906 or cavum concha 907) that the stimulating electrode 31 is in contact with. The taVNS unit 30 may give electrical stimulation in a way similar to transcutaneous electrical nerve stimulation (TENS). The stimulus parameters of the electric stimulation (including strength, pattern, duration, interval, frequency and timing etc.) are pre-determined, such as described in Tables 1 & 2. The taVNS unit 30 contains a stimulating electrode 31 which can deliver electric stimuli to the vagus innervated auricular skin of the wearer 900 that the stimulating electrode 31 is in contact with. The stimulating electrode 31 may be attached to vagus innervated auricular skin so that the electric stimuli can be delivered to the auricular branch of vagus nerve.

The chest-based ECG monitoring system 250 may be in electronic communication 19 with the processing unit 50, 401, through Bluetooth, WiFi, a wired connection, or other electronic connection means. The taVNS unit 30 may also be in communication with the processing unit 50, 401, through Bluetooth or other connection means. The chest-based ECG monitoring system 250 may be configured to transmit the recorded ECG data to the processing unit 50, 401. The processing unit 50, 401, may be configured to analyze the ECG data to detect presence or cessation of any serious cardiac arrhythmia or any impending serious cardiac arrhythmia. When the processing unit 50, 401, detects presence of a serious cardiac arrhythmia, the processing unit 50, 401, may be configured to immediately send arrhythmia-warning-signals to the taVNS unit 30 to automatically start sending pre-determined electric stimuli to the vagus nerve via the stimulating electrode 31 (examples of stimulating parameters as shown in Table 1). When the processing unit 50, 401, detects presence of an impending serious cardiac arrhythmia, the processing unit 50, 401, may be configured to immediately send impending-arrhythmia-warning-signals to the taVNS unit 30 to automatically start sending pre-determined electric stimuli to the vagus nerve via the stimulating electrode 31 (examples of stimulating parameters as shown in Table 2). When the processing unit 50, 401, detects cessation of the serious cardiac arrhythmia or cessation of the impending serious cardiac arrhythmia, the processing unit 50, 401, may be further configured to immediately send arrhythmia-cessation-signals or impending-arrhythmia-cessation signals to the taVNS unit 30 to automatically stop or modify the electric stimuli to the vagus nerve.

In some embodiments, the chest-based automatic anti-arrhythmia system 200B may comprise a network interface 53, 406, and the network interface 53, 406, may be configured to generate an electronic notification via input/output (I/O) interfaces 404 of a client device 400 when the processing unit 50, 401, detects the presence or cessation of a serious cardiac arrhythmia or an impending serious cardiac arrhythmia. For example, the network interface 53, 406, may be configured to generate an electronic notification, such as a text message, email, or other electronic user readable message, to the client device 400 of the wearer 900 of this chest-based automatic anti-arrhythmia system 200B and/or to the client device 400 of the wearer's 900 healthcare provider 950 to take appropriate actions when serious cardiac arrhythmia or impending serious cardiac arrhythmia is detected. In preferred embodiments, the chest-based automatic anti-arrhythmia system 200B may comprise one or more client devices 400 that may be configured to generate a visible notification (through a display screen 404C on a client device 400) when a processing unit 50, 401, detects the presence or cessation of a serious cardiac arrhythmia or an impending serious cardiac arrhythmia, including atrial fibrillation (AFib), atrial flutter (AFL), supraventricular tachycardia (SVT), ventricular tachycardia (VT), and ventricular fibrillation (VF). The chest-based automatic anti-arrhythmia system 200B may include a notification function, such as through a speaker 15, 404A or a vibrator 17, 404B. The speaker 15, 404A (that may be configured to make audible warning notification) or vibrator 17, 404B (that may be configured to give tactile warning notification to the wearer 900) may be configured to give notification when presence of a serious cardiac arrhythmia or an impending serious cardiac arrhythmia is detected by the processing unit 50, 401.

Referring to FIG. 19, an example of a chest-based automatic anti-arrhythmia system 200B, comprising a transcutaneous auricular vagus nerve stimulation (taVNS) unit 30 and a chest-based ECG monitoring system 250, is shown. The taVNS unit 30 is attached to the tragus-concha region 905, 906, 907, of external ear 902 with its stimulating electrode 31 attached to the tragus 905. The taVNS unit 30 and the chest-based ECG monitoring system 250 are both linked with a processing unit 401 of a client device 400 through Bluetooth or other connection means. (Example of a chest-based ECG monitoring system 250: Assert-IQ ICM from Abbott, which is placed subcutaneously in left anterior chest 910).

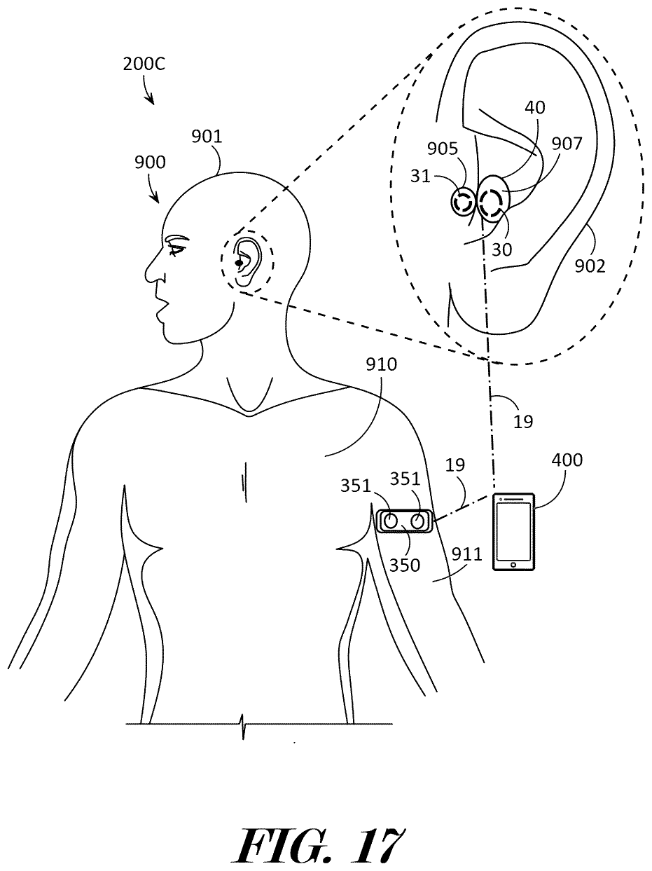

In some embodiments, and referring to FIGS. 17 and 18, an arm-based automatic anti-arrhythmia system 200C may comprise an arm-based ECG monitoring system 350, a transcutaneous auricular vagus verve stimulation (taVNS) unit 30 and a processing unit 50, 401. The arm-based ECG monitoring system 350 comprises an arm-based ECG recording module 356 that is in electronic communication with at least two arm-contacting electrodes 351. The arm-based ECG recording module 356 is configured to record ECG data via the at least two arm-contacting electrodes 351. Example of an arm-based ECG monitoring system 350 includes MUAC armband ECG monitoring from Ulster University, UK. The processing unit 50, 401 may be in communication with the taVNS unit 30 and the arm-based ECG monitoring system 350. The taVNS unit 30 may be housed in an in-the-ear portion 27 of an auricular housing structure 11 and the in-the-ear portion is configured to be placed inside an external ear canal 904 and a bowl-shaped area of tragus-concha 905, 906, 907 of a wearer's ear 902. Alternatively, the taVNS unit 30 may be attached to a location on an external ear 902 or behind the ear 903 (behind the ear portion of a peri-auricular area 903). The taVNS unit 30 may comprise a stimulating electrode 31 which is configured to give pre-determined electric stimuli transcutaneously to vagus innervated auricular skin that the stimulating electrode 31 is in contact with in a way similar to transcutaneous electrical nerve stimulation (TENS). The stimulus parameters of the electric stimulation (including strength, pattern, duration, interval, frequency and timing etc.) are pre-determined, such as described in Tables 1 & 2. The taVNS unit 30 contains a stimulating electrode 31 which can deliver electric stimuli to the vagus innervated auricular skin of the wearer 900 that the stimulating electrode 31 is in contact with. The stimulating electrode 31 may be attached to vagus innervated auricular skin (including tragus 905, concha 906, 907, and external ear canal 904) so that the electric stimuli can be delivered to the auricular branch of vagus nerve.

The arm-based ECG monitoring system 350 may be in electronic communication 19 with a processing unit 50, 401, through Bluetooth, WiFi, a wired connection, or other electronic connection means. The taVNS unit 30 may also be in communication with the processing unit 50, 401, through Bluetooth or other connection means. The arm-based ECG monitoring system 350 may be configured to transmit the recorded ECG data to the processing unit 50, 401. The processing unit 50, 401, may be configured to analyze the ECG data to detect presence or cessation of any serious cardiac arrhythmia or any impending serious cardiac arrhythmia. When the processing unit 50, 401, detects presence of a serious cardiac arrhythmia, the processing unit 50, 401, may be configured to immediately send arrhythmia-warning-signals to the taVNS unit 30 to automatically start sending pre-determined electric stimuli to the vagus nerve via the stimulating electrode 31 (stimulation parameters as shown in Table 1). When the processing unit 50, 401, detects presence of an impending serious cardiac arrhythmia, the processing unit 50, 401, may be configured to immediately send impending-arrhythmia-warning-signals to the taVNS unit 30 to automatically start sending pre-determined electric stimuli to the vagus nerve via the stimulating electrode 31 (stimulation parameters as shown in Table 2). When the processing unit 50, 401, detects cessation of the serious cardiac arrhythmia or cessation of the impending serious cardiac arrhythmia, the processing unit 50, 401, may be further configured to immediately send arrhythmia-cessation-signals or impending-arrhythmia-cessation signals to the taVNS unit 30 to automatically stop or modify the electric stimuli to the vagus nerve.

In some embodiments, the arm-based automatic anti-arrhythmia system 200C may comprise a network interface 53, 406, and the network interface 53, 406, may be configured to generate an electronic notification via input/output (I/O) interfaces 404 of a client device 400 when the processing unit 50, 401, detects the presence or cessation of a serious cardiac arrhythmia or an impending serious cardiac arrhythmia. For example, the network interface 53, 406, may be configured to generate a visible notification (through a display screen 404C on a client device 400), such as a text message, email, or other electronic user readable message, to the client device 400 of the wearer 900 of this arm-based automatic anti-arrhythmia system 200C and/or to the client device 400 of the wearer's 900 healthcare provider 950 to take appropriate actions when a serious cardiac arrhythmia or an impending serious cardiac arrhythmia is detected. In preferred embodiments, the arm-based automatic anti-arrhythmia system 200C may comprise one or more client devices 400 that may be configured to generate a notification when a processing unit 50, 401, detects the presence or cessation of a serious cardiac arrhythmia or an impending serious cardiac arrhythmia, including atrial fibrillation (AFib), atrial flutter (AFL), supraventricular tachycardia (SVT), ventricular tachycardia (VT), and ventricular fibrillation (VF). The arm-based automatic anti-arrhythmia system 200C may further include a notification function, such as through a speaker 15, 404A or a vibrator 17, 404B. The speaker 15, 404A (that may be configured to make audible warning notification) or the vibrator 17, 404B (that may be configured to make tactile notification to the wearer 900) are configured to send notification when the processing unit 50, 401, detects presence of the serious cardiac arrhythmia or the impending serious cardiac arrhythmia.

Referring to FIG. 17, an example of an arm-based automatic anti-arrhythmia system 200C, comprising a transcutaneous auricular vagus nerve stimulator (taVNS) unit 30 and an arm-based ECG monitoring system 350, is shown. The taVNS unit 30 is attached to the tragus-concha region 905, 906, 907, of external ear 902 with its stimulating electrode 31 attached to the tragus 905. The taVNS unit 30 and the arm-based ECG monitoring system 350 are both linked with a processing unit 401 of a client device 400 through Bluetooth or other connection means. (Example of an arm-based ECG monitoring system 350: MUAC armband ECG monitoring from Ulster University, UK, which is placed on left upper arm 911 of a wearer 900).

### A Fourth Preferred Embodiment

According to still another aspect consistent with the principles of the invention, in some embodiments, a novel smart-ring ECG monitoring system 450 may be configured as another example of a wearable self-installable self-removable ECG monitoring system. The smart-ring ECG monitoring system 450 may be configured as an ear-finger ECG system 450A or a finger-finger ECG system 450B. According to yet another aspect consistent with the principles of the invention, in some embodiments, a novel wearable self-installable self-removable smart-ring automatic anti-arrhythmia system 460 may be configured as an ear-finger automatic anti-arrhythmia system 460A or a finger-finger automatic anti-arrhythmia system 460B. The novel ear-finger automatic anti-arrhythmia system 460A may be configured to comprise an ear-finger ECG system 450A, a transcutaneous auricular vagus verve stimulation (taVNS) unit 30, and a processing unit 50, 401. Similarly, the novel finger-finger automatic anti-arrhythmia system 460B may be configured to comprise a finger-finger ECG system 450B, a transcutaneous auricular vagus verve stimulation (taVNS) unit 30, and a processing unit 50, 401, detail to be described hereinafter.

Referring to FIGS. 22 & 24, in some embodiments, an ear-finger ECG system 450A may comprise at least two wired or wireless ECG electrodes 83, 86, in electronic communication (preferably wireless communication) with an ECG recording module 20. The at least two wired or wireless ECG electrodes 83, 86 include a first finger-ring electrode 83 and a first auricular electrode 86. The ECG recording module 20 is configured to record ECG data of the wearer 900 via the at least two wired or wireless ECG electrodes 83, 86, including the first finger-ring electrode 83 and the first auricular electrode 86. The ear-finger ECG system 450A further comprises an amplifier and filter 21, an analog-to-digital converter (ADC) 22, a processing unit 50, a speaker 15 and a vibrator 17 and functions of these components are similar to descriptions hereinbefore. The ECG recording module 20 is in electronic communication with a processing unit 50, 401. The processing unit 50, 401, is configured to analyze the ECG data transmitted from the ECG recording module 20 to assess the ECG profile of the wearer 900 and to detect presence or absence of any serious cardiac arrhythmia or any impending serious cardiac arrhythmia. The first finger-ring electrode 83 is configured to be placed on an inner surface 87 of a first finger-ring 83A to be worn on a finger (for example an X-finger 81A) on a first hand 81 of the wearer 900. The first finger-ring electrode 83 is configured to be located on the inner surface 87 of the first finger-ring 83A and the first finger-ring 83A is configured to be made of an elastic adaptable and flexible material (such as silicone-type material) and the elastic adaptable and flexible material is configured to have appropriate size, elasticity, adaptability and flexibility such that the first finger-ring electrode 83 will snugly contact the skin of the wearer's X-finger 81A when the wearer wears the first finger-ring 83A on the X-finger 81A of the wearer's 900 first hand 81. The first finger-ring electrode 83 is configured to be partially embedded in the inner surface 87 of the first finger-ring 83A with slight protrusion at the inner surface 87 of the first finger-ring 83A such that the first finger-ring electrode 83 will naturally snugly contact the skin of the wearer's X-finger 81A when the first finger-ring 83A is worn on the X-finger 81A of the wearer's 900 first hand 81. The first auricular electrode 86 is configured to be placed on an in-the-ear portion 27 of an auricular housing structure 11 and the in-the-ear portion 27 is configured to be placed inside an external ear canal 904 and a bowl-shaped area of tragus-concha 905, 906, 907, of an ear 902 of the wearer 900 when in use. The auricular housing structure 11 includes an in-the-ear portion 27 that is configured to be in contact with the skin of the external ear canal 904 and skin of the bowl-shaped area of tragus-concha 905, 906, 907 of the wearer's ear 902 when the in-the-ear portion 27 is inserted (or put in place) into a wearer's ear 902. The auricular housing structure 11 may be selected from one of the following: an earbud-style structure 11A, a behind-the-ear-hearing-aid-style structure 11C, or a tubular-shaped structure 11B. The in-the-ear portion 27 of the auricular housing structure 11 is configured to comprise an elastic adaptable and flexible material (such as soft foam or soft silicone-type material.). The first auricular electrode 86 is configured to be partially embedded in a surface 27A of the in-the-ear portion 27 of the auricular housing structure 11 with slight protrusion at the surface 27A of the in-the-ear portion 27. The in-the-ear portion 27 of the auricular housing structure 11 is configured to have appropriate size, elasticity, adaptability and flexibility such that the first auricular electrode 86 will naturally snugly in close contact with the skin of the external ear canal 904 and skin of the bowl-shaped area of tragus-concha 905, 906, 907, of the wearer's ear 902 when the in-the-ear portion 27 is placed into the wearer's ear 902. The ear-finger ECG system 450A may comprise an ECG recording module 20 that may be housed either within the first finger-ring 83A or within the first auricular housing structure 11. The first finger-ring electrode 83 and the first auricular electrode 86 may be configured to be wired or wireless ECG electrodes (preferably wireless electrodes). The ECG recording module 20 may be configured to comprise a wired or wireless ECG amplifier 21 (preferably wireless). The first finger-ring electrode 83 and the first auricular electrode 86 may be configured to be in wired or wireless (preferably wireless) communication with the ECG recording module 20 and the ECG recording module 20 is configured to record ECG data via the first finger-ring electrode 83 and the first auricular electrode 86. The ECG recording module 20 is in electronic communication with a processing unit 50, 401. The processing unit 50, 401, is configured to analyze the ECG data recorded by the ECG recording module 20 to assess the wearer's ECG profile and to detect presence or cessation of a serious cardiac arrhythmia or an impending serious cardiac arrhythmia (including AFib, AFL, SVT, VT and VF). The ear-finger ECG system 450A may further comprise a network interface 53, 406 and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit 50, 401 detects presence of a serious cardiac arrhythmia or presence of an impending serious cardiac arrhythmia, the processing unit 50, 401, is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401 may be configured to send visible notification to a display screen 404C of a client device 400 of the wearer's healthcare provider 950 to alert the healthcare provider 950 to take appropriate action.

Referring to FIGS. 23 & 25, in some embodiments, a finger-finger ECG system 450B may comprise at least two wired or wireless ECG electrodes 83, 84, in electronic communication (preferably wireless communication) with an ECG recording module 20. The at least two wired or wireless ECG electrodes 83, 84 include a first finger-ring electrode 83 and a second finger-ring electrode 84. The ECG recording module 20 is configured to record ECG data of the wearer 900 via the at least two wired or wireless ECG electrodes 83, 86, including the first finger-ring electrode 83 and the second finger-ring electrode 84. The finger-finger ECG system 450B further comprises an amplifier and filter 21, an analog-to-digital converter (ADC) 22, a processing unit 50, a speaker 15 and a vibrator 17 and functions of these components are similar to descriptions hereinbefore. The ECG recording module 20 is in electronic communication with a processing unit 50, 401. The processing unit 50, 401, is configured to analyze the ECG data transmitted from the ECG recording module 20 to assess the ECG profile of the wearer 900 and to detect presence or absence of any serious cardiac arrhythmia or any impending serious cardiac arrhythmia. The first finger-ring electrode 83 is configured to be placed on an inner surface 87 of a first finger-ring 83A to be worn on a finger (for example an X-finger 81A) of the wearer's 900 first hand 81. The second finger-ring electrode 84 is configured to be placed on an inner surface 87 of a second finger-ring 84A that is configured to be worn on a finger (for example a Y-finger 82A) of the wearer's second hand 82. The first finger-ring 83A may be configured to be made of elastic flexible adaptable material (for example silicone-type material) and the elastic flexible adaptable material is configured to have appropriate size, elasticity, flexibility and adaptability such that the first finger-ring 83A will snugly fit the X-finger 81A of the wearer's first hand 81 when the first finger-ring 83A is worn by the wearer 900. The first finger-ring electrode 83 is configured to be partially embedded in the inner surface 87 of the first finger-ring 83A with slight protrusion at the inner surface 87 of the first finger-ring 83A such that the first finger-ring electrode 83 will naturally snugly in close contact with the skin of the X-finger 81A of the wearer's first hand 81 when the first finger-ring 83A is worn by the wearer 900. Likewise, the second finger-ring electrode 84 is configured to be located on an inner surface 87 of the second finger-ring 84A and the second finger-ring 84A is configured to be made of elastic flexible adaptably material and the elastic flexible adaptable material is configured to have appropriate size, flexibility, elasticity and adaptability such that the second finger-ring 84A will snugly fit the Y-finger 82A of the wearer's second hand 82 when the wearer wears the second finger-ring 84A on the wearer's Y-finger 82A. The second finger-ring electrode 84 is configured to be partially embedded in the inner surface 87 of the second finger-ring 84A with slight protrusion at the inner surface 87 of the second finger-ring 84A such that the second finger-ring electrode 84 will naturally snugly in close contact with the skin of the Y-finger 82A of the wearer's second hand 82 when the second finger-ring 84A is worn by the wearer 900. The first finger-ring electrode 83 and the second finger-ring electrode 84 may be configured as wired or wireless (preferably wireless) ECG electrodes. The ECG recording module 20 may be configured to comprise wired or wireless (preferably wireless) ECG amplifier 21. The first finger-ring electrode 83 and the second finger-ring electrode 84 are configured to be in wired or wireless (preferably wireless) communication with the ECG recording module 20 and the ECG recording module 20 is configured to record ECG data via the first finger-ring electrode 83 and the second finger-ring electrode 84. The processing unit 50, 401, is configured to analyze the ECG data recorded by the ECG recording module 20 to detect presence or cessation of any serious cardiac arrhythmia or any impending serious cardiac arrhythmia (including AFib, AFL, SVT, VT and VF). The finger-finger ECG system 450B may further comprise a network interface 53, 406 and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit 50, 401 detects presence of a serious cardiac arrhythmia or an impending serious cardiac arrhythmia, the processing unit 50, 401, is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401 may be configured to send visible notification to a display screen 404C of a client device 400 of the wearer's healthcare provider 950 to alert the healthcare provider 950 to take appropriate action.

In some embodiments, referring to FIG. 24, an ear-finger automatic anti-arrhythmia system 460A is configured to comprise an ear-finger ECG system 450A, a transcutaneous auricular vagus verve stimulation (taVNS) unit 30 and a processing unit 50, 401. The ear-finger ECG system 450A and the taVNS unit 30 are as described hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401 is in electronic communication (preferably wireless communication) with the taVNS unit 30 and the ear-finger ECG system 450A. The processing unit 50, 401 is configured to analyze the ECG data transmitted from the ear-finger ECG system 450A to assess the wearer's ECG profile and to detect presence or cessation of a serious cardiac arrhythmia (including AFib, AFL, SVT, VT, VF and WPW) or an impending serious cardiac arrhythmia. When at least one serious cardiac arrhythmia is detected by the processing unit 50, 401, the processing unit 50, 401, is configured to send arrhythmia-warning-signals to the taVNS unit 30 and upon receiving arrhythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with, similar to descriptions hereinbefore. (Examples for pre-determined electric stimulation parameters for the taVNS unit 30 are shown on Table 1.) When at least one impending serious cardiac arrhythmia is detected by the processing unit 50, 401, the processing unit 50, 401, is further configured to send impending-arrhythmia-warning-signals to the taVNS unit 30 and upon receiving impending-arrhythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with, similar to descriptions hereinbefore. (Examples for pre-determined electric stimulation parameters for the taVNS unit 30 are shown on Table 2.) When cessation of the at least one serious cardiac arrhythmia is detected by the processing unit 50, 401, the processing unit 50, 401 is further configured to send arrhythmia-cessation-signals to the taVNS unit 30 and upon receiving these signals from the processing unit the taVNS unit 30 is configured to stop sending pre-determined electric stimuli. When cessation of the at least one impending serious cardiac arrhythmia is detected by the processing unit 50, 401, the processing unit 50, 401 is further configured to send impending-arrhythmia-cessation signals to the taVNS unit 30 and upon receiving these signals from the processing unit the taVNS unit 30 is configured to stop sending pre-determined electric stimuli. The ear-finger automatic anti-arrhythmia system 460A may further comprise a network interface 53, 406, and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900. When the processing unit 50, 401, detects presence of a serious cardiac arrhythmia or an impending serious cardiac arrhythmia, the processing unit 50, 401 is configured to send signals to the speaker 15, 404A, (that is configured to generate an audible warning notification), the vibrator 17, 404B, (that is configured to generate a tactile warning notification) and the display screen 404C (that is configured to generate a visible warning notification) on the client device 400 of the wearer 900 to alert the wearer 900 to take appropriate action. Additionally, the processing unit 50, 401 may be configured to send visible notification to a display screen 404C of a client device 400 of the wearer's healthcare provider 950 to alert the healthcare provider 950 to take appropriate action. The housing for the first finger-ring electrode 83 and the first auricular electrode 86 is the same as the aforementioned housing for the ear-finger ECG system 450A. The stimulating electrode 31 of the taVNS unit 30 may be housed in a taVNS housing 40 that may be integrated with or part of an auricular housing structure 11. The stimulating electrode may be configured to be partially embedded in a surface 27A of the in-the-ear portion 27 of the auricular housing structure 11 with slight protrusion at the surface 27A of the in-the-ear portion 27 to enable the stimulating electrode 31 to be naturally snugly in close contact with the skin of the external ear canal 904 when the in-the-ear portion 27 is placed into an external ear canal 904 and a bowl-shaped area of tragus-concha 905, 906, 907, of the wearer's ear 902, similar to descriptions hereinbefore. These features will enable the first finger-ring electrode 83, the first auricular electrode 86 and the stimulating electrode 31 of the taVNS unit 30 to be easily self-installable and easily self-removable. These features and setups will also enable smart ring (for example the first finger-ring 83A housing the first finger-ring electrode 83) and the in-the-ear portion 27 of the auricular housing structure 11 (housing the first auricular electrode 86 and the taVNS stimulating electrode 31) of this invention to be another excellent example of self-installable-housing structures.

In some embodiments, referring to FIG. 25, a finger-finger automatic anti-arrhythmia system 460B may be configured to comprise a finger-finger ECG system 450B, a transcutaneous auricular vagus nerve stimulation (taVNS) unit 30 and a processing unit 50, 401. The finger-finger ECG system 450B and the taVNS unit 30 are as described hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401 is in electronic communication (preferably wireless communication) with the transcutaneous auricular vagus verve stimulation (taVNS) unit 30 and the finger-finger ECG system 450B. The processing unit 50, 401 is configured to process the ECG data transmitted from the finger-finger ECG system 450B to assess the wearer's ECG profile and to detect presence or cessation of a serious cardiac arrhythmia or an impending serious cardiac arrhythmia (including AFib, AFL, SVT, VT and VF). When at least one serious cardiac arrhythmia is detected by the processing unit 50, 401, the processing unit 50, 401, is configured to send arrhythmia-warning-signals to the taVNS unit 30 and upon receiving the arrhythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with (examples of stimulating parameters as shown in Table 1). When at least one impending serious cardiac arrhythmia is detected by the processing unit 50, 401, the processing unit 50, 401, is further configured to send impending-arrhythmia-warning-signals to the taVNS unit 30 and upon receiving the impending-arrhythmia-warning signals from the processing unit 50, 401, the taVNS unit 30 is configured to start sending pre-determined electric stimulation to the vagus innervated auricular skin to which the stimulating electrode 31 of the taVNS unit 30 is in contact with (examples of stimulating parameters as shown in Table 2). When cessation of the at least one serious cardiac arrhythmia is detected by the processing unit 50, 401, the processing unit 50, 401 is further configured to send arrhythmia-cessation-signals to the taVNS unit 30 to cause the taVNS unit 30 to stop sending pre-determined electric stimuli. When cessation of the at least one impending serious cardiac arrhythmia is detected by the processing unit 50, 401, the processing unit 50, 401 is further configured to send impending-arrhythmia-cessation signals to the taVNS unit 30 to cause the taVNS unit 30 to stop sending pre-determined electric stimuli. The finger-finger automatic anti-arrhythmia system 460B may further comprise a network interface 53, 406, and one of the following: a speaker 15, 404A, a vibrator 17, 404B, and a display screen 404C on a client device 400 of the wearer 900 or a client device 400 of the wearer's healthcare provider 950, similar to descriptions hereinbefore. The housing of the first finger-ring electrode 83 and the second finger-ring electrode 84 is the same as the aforementioned housing for the finger-finger ECG system 450B. The stimulating electrode 31 of the taVNS unit 30 may be housed in a taVNS housing 40 that may be integrated with or part of an in-the-ear portion 27 of an auricular housing structure 11. The stimulating electrode may be configured to be partially embedded in a surface 27A of the in-the-ear portion 27 of the auricular housing structure 11 with slight protrusion at the surface 27A of the in-the-ear portion 27 to enable the stimulating electrode 31 to be naturally snugly in close contact with the skin of external ear canal 904 or skin of the bowl-shaped area of tragus-concha 905, 906, 907, when the in-the-ear portion 27 is inserted into a wearer's ear 902, similar to descriptions hereinbefore. These features will enable the first finger-ring electrode 83, the second finger-ring electrode 84 and the stimulating electrode 31 of the taVNS unit 30 to be easily self-installable and easily self-removable. These features and setups will also enable smart rings (for example the first finger-ring 83A housing the first finger-ring electrode 83 and second finger-ring 84A housing the second finger-ring electrode 84) and the in-the-ear portion 27 of the auricular housing structure 11 (housing the taVNS stimulating electrode 31) of this invention to be yet another excellent example of self-installable-housing structures. By using self-installable housing structures and self-installable electrodes of this invention, there is no need to have a technologist to pre-prepare the skin surface before attaching an electrode to a wearer's skin and no need to use special adhesive material to secure the electrodes to the wearer's skin.

As used herein, the term “auricular housing structure 11” refers to housing structures in or around the ear. Examples of auricular housing structures include an earbud-style structure 11A, a behind-the-ear-hearing-aid-style structure 11C, a tubular-shaped structure 11B and a taVNS housing 40 used for a taVNS unit 30. Also, as used herein, the term “in-the-ear portion 27” of an auricular housing structure 11 refers to a portion of the auricular housing structure 11 that is placed inside an external ear canal 904 and a bowl-shaped area of tragus-concha 905, 906, 907, when in use. Additionally, as used herein, all of the self-installable self-removable housing structures that are used to house ECG electrodes 12, 13, 88, 83, 84, 86, 73, (such as auricular housing structure 11 and smart rings 83A, 84A, 73A) and taVNS stimulating electrode 31 will be referred collectively as “self-installable-housing structures” herein when their housings have the aforementioned features and setups.

According to yet another aspect consistent with the principles of the invention, in some alternative embodiments, a smart ring with an ECG electrode may be worn on a toe of a foot of the wearer 900. A smart ring (toe-ring) with an ECG electrode (toe-ring electrode) being worn on a toe of the wearer may have all of the similar features and setups as aforementioned descriptions of a smart ring (finger-ring) with an ECG electrode (finger-ring electrode) being worn on a finger of the wearer 900 (for example a first finger-ring electrode 83 located on a first finger-ring 83A worn on an X-finger 81A of the wearer's first hand 81.) Thus, a toe-ring with a toe-ring electrode worn on a toe will be equally self-installable and self-removable. A toe-ring electrode may be in communication with an ECG recording module 20, similar to a finger-ring electrode worn on a finger. These alternative embodiments may include ear-toe ECG monitoring system 750A, finger-toe ECG monitoring system 750B, toe-toe ECG monitoring system, ear-toe automatic anti-arrhythmia system 760A, finger-toe automatic anti-arrhythmia system 760B, and toe-toe automatic anti-arrhythmia system, etc. Additionally, an ECG electrode may be mounted on an ankle band to be worn on an ankle of a wearer or on a wrist band to be worn on a wrist. (Note: an ankle band is usually more stable and less movement interference than a wrist band and thus ankle band is more suitable for continuous monitoring.) All of these alternative systems are within the aspects consistent with the principles of the present invention.

In alternative embodiments, a smart-ring ECG monitoring system 450 may be configured as an ear-toe ECG system 750A or a finger-toe ECG system 750B. In other embodiments, a smart-ring automatic anti-arrhythmia system 460 may be configured as an ear-toe automatic anti-arrhythmia system 760A or a finger-toe automatic anti-arrhythmia system 760B. Referring to FIG. 26, in some embodiments, an ear-toe ECG system 750A may comprise at least two wired or wireless ECG electrodes 73, 86, in electronic communication (preferably wireless communication) with an ECG recording module 20. The at least two wired or wireless ECG electrodes 73, 86 include a first toe-ring electrode 73 and a first auricular electrode 86. The ECG recording module 20 is configured to record ECG data of the wearer 900 via the at least two wired or wireless ECG electrodes 73, 86, including the first toe-ring electrode 73 and the first auricular electrode 86. The ear-toe ECG system 750A further comprises an amplifier and filter 21, an analog-to-digital converter (ADC) 22, a processing unit 50, a speaker 15 and a vibrator 17 and functions of these components are similar to descriptions hereinbefore. The ECG recording module 20 is in electronic communication with a processing unit 50, 401. The processing unit 50, 401, is configured to analyze the ECG data transmitted from the ECG recording module 20 to assess the ECG profile of the wearer 900 and to detect presence or absence of any serious cardiac arrhythmia or any impending serious cardiac arrhythmia. The first toe-ring electrode 73 is configured to be placed on an inner surface 77 of a first toe-ring 73A to be worn on a toe (for example an X-toe 71A) of a wearer's first foot 71. The first toe-ring electrode 73 is configured to be located on an inner surface 77 of the first toe-ring 73A and the first toe-ring 73A is configured to be made of an elastic adaptable and flexible material (such as silicone-type material) and the elastic adaptable flexible material is configured to have appropriate size, elasticity, adaptability and flexibility such that the first toe-ring 73A will snugly contact the skin of the X-toe 71A of the wearer's first foot 71 when the wearer wears the first toe-ring 73A on the X-toe 71A of the wearer's first foot 71. The first toe-ring electrode 73 is configured to be partially embedded in the inner surface 77 of the first toe-ring 73A with slight protrusion at the inner surface 77 of the first toe-ring 73A such that the first toe-ring electrode 73 will naturally snugly contact the skin of the X-toe 71A of the wearer's first foot 71 when the first toe-ring 73A is worn on the X-toe 71A of the wearer's first foot 71. Thus, the first toe-ring electrode 73 can be easily self-installed and self-removed by the wearer (user) and there is no need to have a technologist to perform installation or removal of the first toe-ring electrode 73. The first auricular electrode 86 is configured to be placed on a surface 27A of an in-the-ear portion 27 of an auricular housing structure 11 that is configured to be worn on or in an ear 902 of the wearer 900. The auricular housing structure 11 includes an in-the-ear portion 27 that is configured to be in contact with the wearer's external ear canal 904 and the bowl-shaped area of tragus-concha 905, 906, 907, when the in-the-ear portion 27 is put in place into a wearer's external ear canal 904 and the bowl-shaped area of tragus-concha 905, 906, 907. The auricular housing structure 11 may be selected from one of the following: an earbud-style structure 11A, a behind-the-ear-hearing-aid-style structure 11C, or a tubular-shaped structure 11B. Optionally, a taVNS housing 40 may be configured to be integrated into or with the auricular housing structure 11. The in-the-ear portion 27 of the auricular housing structure 11 is configured to comprise an elastic, adaptable and flexible material (such as soft foam or soft silicone-type material.). The first auricular electrode 86 is configured to be partially embedded in the surface of the in-the-ear portion 27 with slight protrusion at the surface 27A of the in-the-ear portion 27 of the auricular housing structure 11. The in-the-ear portion 27 of the auricular housing structure 11 is configured to have appropriate size, elasticity, adaptability and flexibility such that the in-the-ear portion 27 will naturally snugly fill the interior of the wearer's external ear canal 904 and the bowl-shaped area of tragus-concha 905, 906, 907, when the in-the-ear portion 27 is put in place into the wearer's external ear canal 904 and the bowl-shaped area of tragus-concha 905, 906, 907, and such that the first auricular electrode 86 will naturally snugly contact the skin of the wearer's external ear canal 904 or skin of the bowl-shaped area of tragus-concha 905, 906, 907, when the in-the-ear portion 27 is put in place. Installing and removing the first auricular electrode 86 will be as easy as inserting and removing the in-the-ear portion 27 from the wearer's external ear 902. This feature of easy self-installation and easy self-removal will be very convenient for the wearer. The ear-toe ECG system 750A may further comprise an ECG recording module 20 that may be housed either within the first toe-ring 73A or within the auricular housing structure 11. The first toe-ring electrode 73 and the first auricular electrode 86 are configured to be wired or wireless ECG electrodes (preferably wireless). The ECG recording module 20 is configured to comprise a wired or wireless (preferably wireless) ECG amplifier 21. The first toe-ring electrode 73 and the first auricular electrode 86 are configured to be in wired or wireless (preferably wireless) communication with the ECG recording module 20 and the ECG recording module 20 is configured to record ECG data of the wearer via the first toe-ring electrode 73 and the first auricular electrode 86. The processing unit 50, 401, is configured to analyze the ECG data recorded by the ECG recording module 20 to assess the ECG profile of the wearer 900 and to detect presence or cessation of any serious cardiac arrhythmia (including AFib, AFL, SVT, VT and VF). The processing unit 50, 401, is further configured to analyze the ECG data recorded by the ECG recording module 20 to detect presence or cessation of any impending serious cardiac arrhythmia. The ear-toe ECG system 750A may further comprise a network interface 53, 406, and one of the following: a speaker 15, 404A, a vibrator 17, 404B, and a display screen 404C on a client device 400 of the wearer 900. The network interface 53, 406, is in electronic communication with the processing unit 50, 401, the speaker 15, 404A, the vibrator 17, 404B, and the display screen 404C on a client device 400 of the wearer 900 or on a client device 400 of the wearer's healthcare provider 950. The functions and setups of the network interface 53, 406, the speaker 15, 404A, the vibrator 17, 404B and display screen 404C are similar to descriptions hereinbefore.

Referring to FIG. 27, in some embodiments, a finger-toe ECG system 750B may comprise at least two wired or wireless ECG electrodes 83, 73, in electronic communication (preferably wireless communication) with an ECG recording module 20. The at least two wired or wireless ECG electrodes 83, 73 include a first finger-ring electrode 83 and a first toe-ring electrode 73. The ECG recording module 20 is configured to record ECG data of the wearer 900 via the at least two wired or wireless ECG electrodes 83, 73, including the first finger-ring electrode 83 and the first toe-ring electrode 73. The finger-toe ECG system 750B further comprises an amplifier and filter 21, an analog-to-digital converter (ADC) 22, a processing unit 50, a speaker 15 and a vibrator 17 and functions of these components are similar to descriptions hereinbefore. The ECG recording module 20 is in electronic communication with a processing unit 50, 401. The processing unit 50, 401, is configured to analyze the ECG data transmitted from the ECG recording module 20 to assess the ECG profile of the wearer 900 and to detect presence or absence of any serious cardiac arrhythmia or any impending serious cardiac arrhythmia. The first finger-ring electrode 83 may be configured to be placed on an inner surface 87 of a first finger-ring 83A to be worn on a finger (for example an X-finger 81A) of the wearer's first hand 81. The first toe-ring electrode 73 may be configured to be placed on an inner surface 77 of a first toe-ring 73A that is configured to be worn on a toe (for example an X-toe 71A) of the wearer's first foot 71. The first finger-ring 83A may be configured to be made of elastic flexible adaptable material (for example silicone-type material) and the elastic flexible adaptable material is configured to have appropriate size, elasticity, adaptability and flexibility such that the first finger-ring 83A will snugly fit the X-finger 81A of the wearer's first hand 81 when the wearer wears the first finger-ring 83A on the X-finger 81A of the wearer's first hand 81. The first finger-ring electrode 83 is configured to be partially embedded in the inner surface 87 of the first finger-ring 83A with slight protrusion at the inner surface 87 of the first finger-ring 83A such that the first finger-ring electrode 83 will snugly contact the skin of the X-finger 81A of the wearer's first hand 81 when the first finger-ring 83A is worn by the wearer 900. Likewise, the first toe-ring electrode 73 is configured to be placed on an inner surface 77 of the first toe-ring 73A and the first toe-ring 73A is configured to be made of elastic flexible adaptable material and the elastic flexible adaptable material is configured to have appropriate size, flexibility, adaptability and elasticity such that the first toe-ring electrode 73 will snugly fit the X-toe 71A of the wearer's first foot 71 when the wearer wears the first toe-ring 73A on the X-toe 71A of the wearer's first foot 71. The first toe-ring electrode 73 is configured to be partially embedded in the inner surface 77 of the first toe-ring 73A with slight protrusion at the inner surface 77 of the first toe-ring 73A such that the first toe-ring electrode 73 will naturally contact the skin of the X-toe 71A of the wearer's first foot 71 when the first toe-ring 73A is worn by the wearer. The first finger-ring electrode 83 and the first toe-ring electrode 73 are configured as wired or wireless ECG electrodes (preferably wireless). The ECG recording module 20 is configured to comprise wired or wireless ECG amplifier (preferably wireless). The first finger-ring electrode 83 and the first toe-ring electrode 73 are configured to be in wired or wireless communication (preferably wireless) with the ECG recording module 20 and the ECG recording module 20 is configured to record ECG data of the wearer via the first finger-ring electrode 83 and the first toe-ring electrode 73. The processing unit 50, 401, is configured to analyze the ECG data recorded by the ECG recording module 20 to assess the wearer's ECG profile and to detect presence or cessation of any serious cardiac arrhythmia (including AFib, AFL, SVT, VT, and VF) or any impending serious cardiac arrhythmia. The finger-toe ECG system 750B may further comprise a network interface 53, 406, and one of the following: a speaker 15, 404A, a vibrator 17, 404B, and a display screen 404C on a client device 400 of the wearer 900 and a client device 400 of the wearer's healthcare provider 950. The functions and setups of the network interface 53, 406, the speaker 15, 404A, the vibrator 17, 404B and display screen 404C are similar to descriptions hereinbefore.

Referring to FIG. 26, in some embodiments, an ear-toe automatic anti-arrhythmia system 760A may be configured to comprise an ear-toe ECG system 750A, a transcutaneous auricular vagus verve stimulation (taVNS) unit 30 and a processing unit 50, 401. The ear-toe ECG system 750A and the taVNS unit 30 are as described hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401, is in electronic communication (preferably wireless communication) with the transcutaneous auricular vagus nerve stimulation (taVNS) unit 30 and the ear-toe ECG system 750A. The processing unit 50, 401, is configured to process the ECG data transmitted from the ear-toe ECG system 750A and the processing unit 50, 401, is configured to assess the wearer's ECG profile and to detect presence or cessation of a serious cardiac arrhythmia or an impending serious cardiac arrhythmia (including AFib, AFL, SVT, VT, and VF). When at least one serious cardiac arrhythmia is detected by the processing unit 50, 401, the processing unit 50, 401, is configured to send signals (arrhythmia-warning signals) to the taVNS unit to prompt or cause (prompted by the arrhythmia-warning signals) the taVNS unit to start sending pre-determined electric stimulation to the vagus innervated auricular skin to which the stimulating electrode 31 of the taVNS unit 30 is in contact with, similar to descriptions hereinbefore. (Examples for stimulation parameters for serious cardiac arrhythmia as shown on Table 1.) When at least one impending serious cardiac arrhythmia is detected by the processing unit 50, 401, the processing unit 50, 401, is further configured to send signals (impending-arrhythmia-warning signals) to the taVNS unit 30 to prompt the taVNS unit 30 to start sending pre-determined electric stimulation to the vagus innervated auricular skin to which the stimulating electrode 31 of the taVNS unit 30 is in contact with, similar to descriptions hereinbefore. (Examples for stimulation parameters for impending serious cardiac arrhythmia are shown on Table 2.) When cessation of the at least one serious cardiac arrhythmia is detected by the processing unit 50, 401, the processing unit 50, 401, is further configured to send signals (arrhythmia-cessation signals) to the taVNS unit 30 to cause the taVNS unit 30 to stop or modify sending pre-determined electric stimuli. When cessation of the at least one impending serious cardiac arrhythmia is detected by the processing unit 50, 401, the processing unit 50, 401, is further configured to send signals (impending-arrhythmia-cessation signals) to the taVNS unit 30 to cause the taVNS unit 30 to stop or modify sending pre-determined electric stimuli. The ear-toe automatic anti-arrhythmia system 760A may further comprise a network interface 53, 406, and one of the following: a speaker 15, 404A, a vibrator 17, 404B, and a display screen 404C on a client device 400 of the wearer 900 or on a client device of the wearer's healthcare provider 950. The functions and setups of the network interface 53, 406, the speaker 15, 404A, the vibrator 17, 404B and display screen 404C are similar to descriptions hereinbefore.

In some embodiments, and referring to FIG. 27, a finger-toe automatic anti-arrhythmia system 760B may be configured to comprise a finger-toe ECG system 750B, a transcutaneous auricular vagus verve stimulation (taVNS) unit 30 and a processing unit 50, 401. The finger-toe ECG system 750B and the taVNS unit 30 are as described hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401, is in electronic communication (preferably wireless communication) with the taVNS unit 30 and the finger-toe ECG system 750B. The processing unit 50, 401, is configured to process the ECG data transmitted from the finger-toe ECG system 750B and the processing unit 50, 401, is configured to assess the wearer's ECG profile and to detect presence or cessation of a serious cardiac arrhythmia or an impending serious cardiac arrhythmia (including AFib, AFL, SVT, VT and VF). When at least one serious cardiac arrhythmia is detected by the processing unit 50, 401, the processing unit 50, 401, is configured to send signals (arrhythmia-warning signals) to the taVNS unit 30 to cause the taVNS unit 30 to start sending pre-determined electric stimulation to the vagus innervated auricular skin to which the stimulating electrode 31 of the taVNS unit 30 is in contact with (stimulating parameters as shown in Table 1). When at least one impending serious cardiac arrhythmia is detected by the processing unit 50, 401, the processing unit 50, 401, is further configured to send signals (impending-arrhythmia-warning signals) to the taVNS unit to cause the taVNS unit to start sending pre-determined electric stimulation to the vagus innervated auricular skin to which the stimulating electrode 31 of the taVNS unit 30 is in contact with (stimulating parameters as shown in Table 2). When cessation of the at least one serious cardiac arrhythmia is detected by the processing unit, the processing unit is configured to send signals (arrhythmia-cessation signals) to the taVNS unit to cause the taVNS unit to stop or modify sending pre-determined electric stimuli. When cessation of the at least one impending serious cardiac arrhythmia is detected by the processing unit, the processing unit is further configured to send signals (impending-arrhythmia-cessation signals) to the taVNS unit to cause the taVNS unit to stop or modify sending pre-determined electric stimuli. The finger-toe automatic anti-arrhythmia system 760B may further comprise a network interface 53, 406, and one of the following: a speaker 15, 404A, a vibrator 17, 404B, and a display screen 404C on a client device 400 of the wearer 900 or a client device 400 of the wearer's healthcare provider 950. The functions and setups of the network interface 53, 406, the speaker 15, 404A, the vibrator 17, 404B and display screen 404C are similar to descriptions hereinbefore.

In some alternative embodiments, a toe-toe ECG system may comprise at least two wired or wireless ECG electrodes in electronic communication (preferably wireless communication) with an ECG recording module 20. The at least two wired or wireless ECG electrodes include a first toe-ring electrode and a second toe-ring electrode. The first toe-ring electrode is configured to be housed in a first toe-ring to be worn on an X-toe of a wearer's first foot. The second toe-ring electrode is configured to be housed in a second toe-ring to be worn on a Y-toe of the wearer's second foot. The ECG recording module 20 is configured to record ECG data of the wearer 900 via the at least two wired or wireless ECG electrodes, including the first toe-ring electrode and the second toe-ring electrode. The toe-toe ECG system further comprises an amplifier and filter 21, an analog-to-digital converter (ADC) 22, a processing unit 50, a speaker 15 and a vibrator 17 and functions of these components are similar to descriptions hereinbefore. The functions and the housing setups of the toe-toe ECG system is essentially similar to the aforementioned descriptions (for example, similar to those for a finger-finger ECG system 450B). For simplicity purpose, figures for the toe-toe ECG system are omitted from the Figures.

In some alternative embodiments, a toe-toe automatic anti-arrhythmia system may be configured to comprise a toe-toe ECG system, a transcutaneous auricular vagus verve stimulation (taVNS) unit 30 and a processing unit 50, 401. The toe-toe ECG system and the taVNS unit 30 are as described hereinbefore. The taVNS unit 30 comprises a stimulating electrode 31 that is configured to contact vagus-innervated auricular skin (including one of: tragus 905, cavum concha 907, cymba concha 906 and external ear canal 904). The processing unit 50, 401, is in electronic communication (preferably wireless communication) with the taVNS unit 30 and the toe-toe ECG system. The processing unit 50, 401, is configured to process the ECG data transmitted from the toe-toe ECG system and the processing unit 50, 401, is configured to assess the wearer's ECG profile and to detect presence or cessation of a serious cardiac arrhythmia or an impending serious cardiac arrhythmia (including AFib, AFL, SVT, VT and VF). When at least one serious cardiac arrhythmia is detected by the processing unit 50, 401, the processing unit 50, 401, is configured to send signals (arrhythmia-warning signals) to the taVNS unit 30 to cause the taVNS unit 30 to start sending pre-determined electric stimulation to the vagus innervated auricular skin to which the stimulating electrode 31 of the taVNS unit 30 is in contact with (stimulating parameters as shown in Table 1). When at least one impending serious cardiac arrhythmia is detected by the processing unit 50, 401, the processing unit 50, 401, is further configured to send signals (impending-arrhythmia-warning signals) to the taVNS unit to cause the taVNS unit to start sending pre-determined electric stimulation to the vagus innervated auricular skin to which the stimulating electrode 31 of the taVNS unit 30 is in contact with (stimulating parameters as shown in Table 2). When cessation of the at least one serious cardiac arrhythmia is detected by the processing unit, the processing unit is configured to send signals (arrhythmia-cessation signals) to the taVNS unit to cause the taVNS unit to stop or modify sending pre-determined electric stimuli. When cessation of the at least one impending serious cardiac arrhythmia is detected by the processing unit, the processing unit is further configured to send signals (impending-arrhythmia-cessation signals) to the taVNS unit to cause the taVNS unit to stop or modify sending pre-determined electric stimuli. The functions and the housing setups of the toe-toe automatic anti-arrhythmia system is essentially similar to the aforementioned descriptions (for example, similar to those for a finger-finger automatic anti-arrhythmia system 460B). For simplicity purpose, figures for toe-toe automatic anti-arrhythmia system are omitted from the Figures.

Most of the ECG monitoring systems (including 100A, 100B, 450A, 450B, 750A, 750B) and automatic anti-arrhythmia systems (including 200A, 460A, 460B, 760A, 760B) in this invention have the aforementioned features of self-installable-housing structures with easy self-installable self-removable electrodes. All of these systems are fully wearable and the wearer can ambulate freely and are very convenient for repeatedly at-home use. For example, auricular ECG monitoring system 100 (including single-ear ECG system 100A and dual-ear ECG system 100B) and the smart-ring ECG monitoring systems 450 (including the ear-finger ECG system 450A, the finger-finger ECG system 450B, the ear-toe ECG system 750A and finger-toe ECG system 750B) contain self-installable and self-removable ECG electrodes 12, 13, 88, 83, 84, 86, 73, and these systems are wearable, due to the set-ups and features of their housing structures (features including being made with flexible elastic adaptable material and electrodes being partially embedded in the surface with slight protrusion at the surface of the electrode-housing structures 11, 83A, 84A, 73A). Likewise, the auricular automatic anti-arrhythmia system 200A, the ear-finger automatic anti-arrhythmia system 460A, finger-finger automatic anti-arrhythmia system 460B, the ear-toe automatic anti-arrhythmia system 760A, the finger-toe automatic anti-arrhythmia system 760B and the taVNS unit 30 also contains self-installable self-removable ECG electrodes 12, 13, 88, 83, 84, 86, 73, and self-installable self-removable taVNS stimulating electrode 31 and these systems are fully wearable, self-installable and self-removable. The auricular housing structures 11, 11A, 11B, 11C, and the smart-ring housing 83A, 84A, 73A, are collectively referred as self-installable-housing structures. These systems are fully wearable, self-installable, self-removable and very convenient for long-term ambulatory use at home.

It should also be noted that a stimulating electrode 31 of a taVNS unit 30 contacting the skin of inner tragus 905 or concha 906, 907 is equally self-installable and self-removable as compared with a stimulating electrode 31 contacting an external ear canal 904. The inner tragus 905 and the concha 906, 907, form a bowl-shaped structure surrounding the opening of an external ear canal 904. Due to the bowl-shaped structure, it is equally feasible that an auricular housing structure 11 will naturally and snugly fill the entire bowl-shaped structure if the auricular housing structure 11 is configured to be made with elastic flexible and adaptable material with appropriate elasticity, flexibility and adaptability. Auricular ECG electrodes 12, 13, 88, 86, and stimulating electrode 31 of a taVNS unit 30 may be configured to be partially embedded in a surface 27A of the auricular housing structure 11 with slight protrusion at the surface 27A of the auricular housing structure 11 to enable these electrodes to be naturally snugly contacting the skin of the inner tragus 905 or concha 906, 907, when the auricular housing structure 11 is put into this bowel-shaped structure, similar to descriptions hereinbefore.

The taVNS stimulation is more often given to the left auricular branch of vagus nerve. There are some studies showing that bilateral taVNS stimulation is safe and could be more effective. However, other studies comparing unilateral versus bilateral taVNS stimulation have shown mixed results. In some embodiments, an automatic anti-arrhythmia system (including 200A, 200B, 200C, 460A, 460B, 760A, 760B) may comprise or utilize a single taVNS unit 30 (preferably wearing on or in the left ear 902) although two taVNS units 30 may be utilized as an option. For most patients or wearers 900 with cardiac arrythmia, who wear an automatic anti-arrhythmia system (200A, 200B, 200C, 460A, 460B, 760A, 760B) that has one taVNS unit 30, the taVNS unit 30 will be placed in or around one ear 902 (preferably the left ear 902). However, optionally, if one-sided (preferably left sided) vagus nerve stimulation is not enough to achieve the desired therapeutic effect for a cardiac arrhythmia or an impending cardiac arrhythmia, the automatic anti-arrhythmia system 200A, 200B, 200C, 460A, 460B, 760A, 760B, may utilize or comprise two taVNS units 30, with one stimulating electrode 31 of a first taVNS unit 30 attached to the vagus innervated auricular skin of one external ear 902 and with another stimulating electrode 31 of a second taVNS unit 30 attached to the vagus innervated auricular skin of the other external ear 902, similar to descriptions hereinbefore.

The automatic anti-arrhythmia systems (including 200A, 200B, 200C, 460A, 460B, 760A, 760B) of this invention are all wearable and will allow the wearer (user) to ambulate freely. They can be easily self-installed and self-removed and are thus very convenient for repeated daily use at home. These features are very valuable for long-term continuous monitoring purpose (systems 100A, 100B, 450A, 450B, 750A, 750B) and, more importantly, for long-term continuous monitoring plus instant automatic therapeutic intervention (systems 200A, 460A, 460B, 760A, 760B) when therapeutic intervention (with immediate automatic vagal stimulation) is urgently needed for suddenly appeared dangerous cardiac arrhythmias or impending serious cardiac arrhythmias.

While some exemplary shapes and sizes have been provided for elements of the ECG monitoring systems 100A, 100B, 450A, 450B, 750A, 750B, and automatic anti-arrhythmia systems 200A, 200B, 200C, 460A, 460B, 760A, 760B, it should be understood to one of ordinary skill in the art that the elements described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

1. An auricular automatic anti-arrhythmia system comprising:
an auricular electrocardiogram (ECG) monitoring system, comprising an ECG recording module, wherein the ECG recording module comprises at least two wired or wireless ECG electrodes configured to contact separate locations of a wearer's skin, wherein the at least two wired or wireless ECG electrodes include a first ECG electrode, wherein the first ECG electrode of the at least two wired or wireless ECG electrodes is configured to be coupled to a first ear of the wearer or a peri-auricular area around the wearer's first ear, wherein the ECG recording module is in electronic communication with each ECG electrode of the at least two wired or wireless ECG electrodes, wherein when the first ECG electrode of the at least two wired or wireless ECG electrodes is coupled to the wearer's first ear or the peri-auricular area around the wearer's first ear the first ECG electrode of the at least two wired or wireless ECG electrodes is positioned in contact with an area of the wearer's skin, the area selected from at least one of the following: an external ear of the wearer's first ear, an external ear canal of the wearer's first ear, and the peri-auricular area around the wearer's first ear, and wherein the ECG recording module is configured to record ECG data of the wearer via the at least two wired or wireless ECG electrodes including the first ECG electrode;
a first transcutaneous auricular vagus nerve stimulation unit (first taVNS unit), the first taVNS unit having a first stimulating electrode to be coupled to the wearer's first ear when in use, wherein the first stimulating electrode is configured to contact vagus innervated auricular skin of the wearer's first ear, and wherein the vagus innervated auricular skin that the first stimulating electrode is configured to contact is selected from at least one of the following: external ear canal of the wearer's first ear, tragus of the wearer's first ear, cymba concha of the wearer's first ear and cavum concha of the wearer's first ear; and
a processing unit in electronic communication with the auricular ECG monitoring system and the first taVNS unit,
i. wherein the processing unit is configured to analyze the ECG data of the wearer recorded by the auricular ECG monitoring system to assess the wearer's ECG profile and to detect the presence or cessation of ECG data suggestive of at least one serious cardiac arrhythmia, wherein the processing unit is further configured to analyze the ECG data recorded by the auricular ECG monitoring system to assess the wearer's ECG profile and to detect the presence or cessation of ECG signals suggestive of at least one impending serious cardiac arrhythmia;
ii. wherein when presence of ECG data suggestive of at least one serious cardiac arrhythmia is detected by the processing unit, the processing unit is configured to immediately send arrhythmia-warning-signals to the first taVNS unit and upon receiving the arrhythmia-warning-signals from the processing unit the first taVNS unit is configured to automatically start sending pre-determined electric stimuli to the vagus innervated auricular skin of the wearer's first ear to which the first stimulating electrode is in contact with;
iii. wherein when cessation of ECG signals suggestive of the at least one serious cardiac arrhythmia is detected by the processing unit, the processing unit is further configured to immediately send arrhythmia-cessation-signals to the first taVNS unit and upon receiving arrhythmia-cessation signals from the processing unit the first taVNS unit is configured to automatically stop sending pre-determined electric stimuli to the vagus innervated auricular skin of the wearer's first ear to which the first stimulating electrode is in contact with;
iv. wherein when presence of ECG data suggestive of at least one impending serious cardiac arrhythmia is detected by the processing unit, the processing unit is configured to immediately send impending-arrhythmia-warning-signals to the first taVNS unit and upon receiving the impending-arrhythmia-warning-signals from the processing unit the first taVNS unit is configured to automatically start sending pre-determined electric stimuli to the vagus innervated auricular skin of the wearer's first ear to which the first stimulating electrode is in contact with; and
v. wherein when cessation of ECG signals suggestive of the at least one impending serious cardiac arrhythmia is detected by the processing unit, the processing unit is further configured to immediately send impending-arrhythmia-cessation-signals to the first taVNS unit and upon receiving impending-arrhythmia-cessation signals from the processing unit the first taVNS unit is configured to automatically stop sending pre-determined electric stimuli to the vagus innervated auricular skin of the wearer's first ear to which the first stimulating electrode is in contact with.

2. The auricular automatic anti-arrhythmia system of claim 1, wherein the auricular ECG monitoring system is configured as a single-ear auricular ECG system, wherein the at least two wired or wireless ECG electrodes further include a second ECG electrode, wherein the first ECG electrode and the second ECG electrode of the at least two wired or wireless ECG electrodes are configured to be coupled to the wearer's first ear or the peri-auricular area around the wearer's first ear, wherein the ECG recording module is in electronic communication with the first ECG electrode and the second ECG electrode, wherein when the first ECG electrode and the second ECG electrode of the at least two wired or wireless ECG electrodes are coupled to the wearer's first ear or the peri-auricular area around the wearer's first ear the first ECG electrode and the second ECG electrode of the at least two wired or wireless ECG electrodes are positioned in contact with separate skin locations of the wearer's skin selected from the following: an external ear of the wearer's first ear, an external ear canal of the wearer's first ear, and the peri-auricular area around the wearer's first ear, and wherein the ECG recording module is configured to record ECG data of the wearer via at least the first ECG electrode and the second ECG electrode.

3. The auricular automatic anti-arrhythmia system of claim 1, wherein the auricular ECG monitoring system is configured as a dual-ear auricular ECG system, wherein the at least two wired or wireless ECG electrodes further include a contralateral ECG electrode, wherein at least the first ECG electrode of the at least two wired or wireless ECG electrodes is configured to be coupled to the wearer's first ear or the peri-auricular area around the wearer's first ear, wherein at least the contralateral ECG electrode of the at least two wired or wireless ECG electrodes is configured to be coupled to a second ear of the wearer or a peri-auricular area around the wearer's second ear, wherein the ECG recording module is in electronic communication with the first ECG electrode and the contralateral ECG electrode of the at least two wired or wireless ECG electrodes, wherein when the contralateral ECG electrode of the at least two wired or wireless ECG electrodes is coupled to the wearer's second ear or the peri-auricular area around the wearer's second ear the contralateral ECG electrode of the at least two wired or wireless ECG electrodes is positioned in contact with an area of the wearer's skin, the area selected from at least one of the following: an external ear of the wearer's second ear, an external ear canal of the wearer's second ear, and the peri-auricular area around the wearer's second ear, and wherein the ECG recording module is configured to record ECG data of the wearer via at least the first ECG electrode and the contralateral ECG electrode.

4. The auricular automatic anti-arrhythmia system of claim 2, wherein the first ECG electrode and the second ECG electrode of the at least two wired or wireless ECG electrodes and the first stimulating electrode of the first taVNS unit are housed in a first in-the-ear portion of a first auricular housing structure, wherein the first in-the-ear portion of the first auricular housing structure is configured to be placed inside the external ear canal and a bowl-shaped area of tragus-concha of the wearer's first ear when in use, wherein the first ECG electrode and the second ECG electrode of the at least two wired or wireless ECG electrodes and the first stimulating electrode of the first ta VNS unit are partially embedded in a surface of the first in-the-ear portion of the first auricular housing structure with slight protrusion at the surface of the first in-the-ear portion, wherein the first in-the-ear portion comprises a flexible elastic and adaptable material and the flexible elastic and adaptable material is configured to have appropriate flexibility, elasticity and adaptability such that the first in-the-ear portion will naturally adapt to the contour of the external ear canal and contour of the bowl-shaped area of tragus-concha of the wearer's first ear and snugly fill the interior of the external ear canal and the bowl-shaped area of tragus-concha of the wearer's first ear when the first in-the-ear portion is placed into the external ear canal and the bowl-shaped area of tragus-concha of the wearer's first ear and such that the first ECG electrode and the second ECG electrode of the at least two wired or wireless ECG electrodes and the first stimulating electrode of the first ta VNS unit are naturally and snugly in contact with the skin of the external ear canal and skin of the bowl-shaped area of tragus-concha of the wearer's first ear when the first in-the-ear portion is placed into the wearer's first ear, wherein the first ECG electrode and the second ECG electrode of the at least two wired or wireless ECG electrodes are configured as wireless ECG electrodes, wherein the ECG recording module comprises a wireless ECG amplifier, wherein the first wireless ECG electrode and the second wireless ECG electrode of the at least two wired or wireless ECG electrodes are housed in the first in-the-ear portion of the first auricular housing structure, wherein the ECG recording module and the processing unit are housed in one of the following: a wearable watch-type structure and a portable smart-phone-type structure, wherein the auricular automatic anti-arrhythmia system further comprises a network interface and at least one of the following: a speaker, a vibrator, and a display screen on a client device of the wearer, wherein the network interface is in electronic communication with the processing unit, the speaker, the vibrator, and the display screen on the client device of the wearer, wherein the network interface is configured to generate a visible notification to the display screen on the client device of the wearer describing the wearer's electrocardiographic profile, wherein the network interface is configured to generate an audible notification to the speaker when the processing unit detects presence of a serious cardiac arrhythmia or presence of an impending serious cardiac arrhythmia, and wherein the network interface is further configured to generate a tactile notification to the vibrator when the processing unit detects presence of a serious cardiac arrhythmia or an impending serious cardiac arrhythmia.

5. The auricular automatic anti-arrhythmia system of claim 3, wherein the first ECG electrode of the at least two wired or wireless ECG electrodes and the first stimulating electrode of the first taVNS unit are housed in a first in-the-ear portion of a first auricular housing structure, wherein the first in-the-ear portion of the first auricular housing structure is configured to be placed inside the external ear canal and a bowl-shaped area of tragus-concha of the wearer's first ear when in use, wherein the first ECG electrode of the at least two wired or wireless ECG electrodes and the first stimulating electrode of the first taVNS unit are partially embedded in a surface of the first in-the-ear portion of the first auricular housing structure with slight protrusion at the surface of the first in-the-ear portion, wherein the first in-the-ear portion comprises a flexible elastic and adaptable material and the flexible elastic and adaptable material is configured to have appropriate flexibility, elasticity and adaptability such that the first in-the-ear portion will naturally adapt to the contour of the external ear canal and contour of the bowl-shaped area of tragus-concha of the wearer's first ear and snugly fill the interior of the external ear canal and bowl-shaped area of tragus-concha of the wearer's first ear when the first in-the-ear portion is placed into the external ear canal and the bowl-shaped area of tragus-concha of the wearer's first ear and such that the first ECG electrode of the at least two wired or wireless ECG electrodes and the first stimulating electrode of the first taVNS unit are naturally and snugly in contact with the skin of the external ear canal and skin of the bowl-shaped area of tragus-concha of the wearer's first ear when the first in-the-ear portion is placed in the external ear canal and the bowl-shaped area of tragus-concha of the wearer's first ear, wherein the contralateral ECG electrode of the at least two wired or wireless ECG electrodes is housed in a second in-the-ear portion of a second auricular housing structure, wherein the second in-the-ear portion of the second auricular housing structure is configured to be placed inside an external ear canal and a bowl-shaped area of tragus-concha of the wearer's second ear when in use, wherein the contralateral ECG electrode of the at least two wired or wireless ECG electrodes is partially embedded in a surface of the second in-the-ear portion with slight protrusion at the surface of the second in-the-ear portion, wherein the second in-the-ear portion comprises a flexible elastic and adaptable material and the flexible elastic and adaptable material is configured to have appropriate flexibility, elasticity and adaptability such that the second in-the-ear portion will naturally adapt to the contour of the external ear canal and contour of the bowl-shaped area of tragus-concha of the wearer's second ear and snugly fill the interior of the external ear canal and bowl-shaped area of tragus-concha of the wearer's second ear when the second in-the-ear portion is inserted into the external ear canal and bowl-shaped area of tragus-concha of the wearer's second ear and such that the contralateral ECG electrode of the at least two wired or wireless ECG electrodes is naturally and snugly in contact with the skin of the external ear canal and skin of the bowl-shaped area of tragus-concha of the wearer's second ear when the second in-the-ear portion is placed into the wearer's second ear, wherein the first ECG electrode and the contralateral ECG electrode of the at least two wired or wireless ECG electrodes are configured as wireless ECG electrodes, wherein the ECG recording module comprises a wireless ECG amplifier, wherein at least the first wireless ECG electrode of the at least two wired or wireless ECG electrodes is housed in the first auricular housing structure, wherein at least the contralateral wireless ECG electrode of the at least two wired or wireless ECG electrodes is housed in the second auricular housing structure, wherein the ECG recording module and the processing unit are housed in one of the following: a wearable watch-type structure and a portable smart-phone-type structure.

6. The auricular automatic anti-arrhythmia system of claim 1, further comprising a second transcutaneous auricular vagus nerve stimulation unit (second taVNS unit), wherein the second taVNS unit comprises a second stimulating electrode to be coupled to the wearer's second ear when in use, wherein the second stimulating electrode is configured to contact vagus innervated auricular skin of the wearer's second ear, wherein the vagus innervated auricular skin that the second stimulating electrode is configured to contact is selected from at least one of the following: external ear canal of the wearer's second ear, tragus of the wearer's second ear, cymba concha of the wearer's second ear and cavum concha of the wearer's second ear, wherein the second taVNS unit is in electronic communication with the processing unit, wherein when presence of ECG signals suggestive of at least one serious cardiac arrhythmia is detected by the processing unit, the processing unit is configured to immediately send arrhythmia-warning-signals to the second taVNS unit and the second taVNS unit is configured to automatically start sending pre-determined electric stimuli to the vagus innervated auricular skin of the wearer's second ear to which the second stimulating electrode is in contact with, wherein when presence of ECG signals suggestive of at least one impending serious cardiac arrhythmia is detected by the processing unit, the processing unit is further configured to immediately send impending-arrhythmia-warning-signals to the second ta VNS unit and the second taVNS unit is configured to automatically start sending pre-determined electric stimuli to the vagus innervated auricular skin of the wearer's second ear to which the second stimulating electrode is in contact with, wherein when cessation of ECG signals suggestive of the at least one serious cardiac arrhythmia is detected by the processing unit, the processing unit is configured to immediately send arrhythmia-cessation-signals to the second taVNS unit and the second taVNS unit is configured to automatically stop the electric stimuli to the vagus innervated auricular skin of the wearer's second ear to which the second stimulating electrode is in contact with, and wherein when cessation of ECG signals suggestive of the at least one impending serious cardiac arrhythmia is detected by the processing unit, the processing unit is configured to immediately send impending-arrhythmia-cessation signals to the second taVNS unit and the second taVNS unit is configured to automatically stop the electric stimuli to the vagus innervated auricular skin of the wearer's second ear to which the second stimulating electrode is in contact with.

7. A smart-ring automatic anti-arrhythmia system, comprising:
a smart-ring electrocardiogram (ECG) monitoring system comprising an ECG recording module and at least two wired or wireless ECG electrodes, wherein the at least two wired or wireless ECG electrodes are configured to contact separate locations of the wearer's skin, wherein the at least two wired or wireless ECG electrodes include a first ring electrode, wherein the first ring electrode is configured to be housed in a ring to be worn on one of: a finger of the wearer and a toe of the wearer, wherein each ECG electrode of the at least two wired or wireless ECG electrodes is in electronic communication with the ECG recording module, and wherein the ECG recording module is configured to record ECG data of the wearer via the at least two wired or wireless ECG electrodes including the first ring electrode;
a first transcutaneous auricular vagus nerve stimulation unit (first taVNS unit), the first taVNS unit having a first stimulating electrode to be coupled to the wearer's first ear when in use, wherein the first stimulating electrode is configured to contact vagus innervated auricular skin of the wearer's first ear, and wherein the vagus innervated auricular skin that the first stimulating electrode is configured to contact is selected from at least one of the following: external ear canal of the wearer's first ear, tragus of the wearer's first ear, cymba concha of the wearer's first ear and cavum concha of the wearer's first ear; and
a processing unit in electronic communication with the smart-ring ECG monitoring system and the first taVNS unit,
i. wherein the processing unit is configured to analyze the ECG data of the wearer recorded by the smart-ring ECG monitoring system to assess the wearer's ECG profile and to detect the presence or cessation of ECG data suggestive of at least one serious cardiac arrhythmia, wherein the processing unit is further configured to analyze the ECG data recorded by the smart-ring ECG monitoring system to assess the wearer's ECG profile and to detect the presence or cessation of ECG signals suggestive of at least one impending serious cardiac arrhythmia;
ii. wherein when presence of ECG data suggestive of at least one serious cardiac arrhythmia is detected by the processing unit, the processing unit is configured to immediately send arrhythmia-warning-signals to the first taVNS unit and upon receiving the arrhythmia-warning-signals from the processing unit the first taVNS unit is configured to automatically start sending pre-determined electric stimuli to the vagus innervated auricular skin of the wearer's first ear to which the first stimulating electrode is in contact with;
iii. wherein when cessation of ECG signals suggestive of the at least one serious cardiac arrhythmia is detected by the processing unit, the processing unit is further configured to immediately send arrhythmia-cessation-signals to the first taVNS unit and upon receiving arrhythmia-cessation signals from the processing unit the first taVNS unit is configured to automatically stop sending pre-determined electric stimuli to the vagus innervated auricular skin of the wearer's first ear to which the first stimulating electrode is in contact with;
iv. wherein when presence of ECG data suggestive of at least one impending serious cardiac arrhythmia is detected by the processing unit, the processing unit is configured to immediately send impending-arrhythmia-warning-signals to the first taVNS unit and upon receiving the impending-arrhythmia-warning-signals from the processing unit the first taVNS unit is configured to automatically start sending pre-determined electric stimuli to the vagus innervated auricular skin of the wearer's first ear to which the first stimulating electrode is in contact with; and
v. wherein when cessation of ECG signals suggestive of the at least one impending serious cardiac arrhythmia is detected by the processing unit, the processing unit is further configured to immediately send impending-arrhythmia-cessation-signals to the first taVNS unit and upon receiving impending-arrhythmia-cessation signals from the processing unit the first taVNS unit is configured to automatically stop sending pre-determined electric stimuli to the vagus innervated auricular skin of the wearer's first ear to which the first stimulating electrode is in contact with.

8. The smart-ring automatic anti-arrhythmia system of claim 7, wherein the smart-ring electrocardiogram (ECG) monitoring system is configured as an ear-finger ECG system, wherein the ear-finger ECG system comprises an ECG recording module, wherein the first ring electrode of the at least two wired or wireless ECG electrodes is configured as a first finger-ring electrode to be housed in a first finger-ring to be worn on an X-finger of a wearer's first hand, wherein the at least two wired or wireless ECG electrodes further include a first auricular electrode, wherein the first auricular electrode is configured to be housed in a first in-the-ear portion of a first auricular housing structure, wherein the first in-the-ear portion of the first auricular housing structure is configured to be placed inside the external ear canal and a bowl-shaped area of tragus-concha of the wearer's first ear when in use, wherein the ECG recording module is configured to comprise a wired or wireless amplifier, wherein the first finger-ring electrode and the first auricular electrode are configured as wireless electrodes, wherein the first finger-ring electrode and the first auricular electrode are in wireless communication with the ECG recording module, wherein the ECG recording module is configured to record ECG data of the wearer via at least the first finger-ring electrode and the first auricular electrode, wherein the processing unit is in electronic communication with the ECG recording module, and wherein the processing unit is further configured to analyze the ECG data recorded by the ECG recording module to assess the wearer's ECG profile and to detect presence or cessation of a serious cardiac arrhythmia and presence or cessation of an impending serious cardiac arrhythmia.

9. The smart-ring automatic anti-arrhythmia system of claim 7, wherein the smart-ring electrocardiogram (ECG) monitoring system is configured as a finger-finger ECG system, wherein the finger-finger ECG system comprises an ECG recording module, wherein the first ring electrode of the at least two wired or wireless ECG electrodes is configured as a first finger-ring electrode to be housed in a first finger-ring to be worn on an X-finger of a wearer's first hand, wherein the at least two wired or wireless ECG electrodes further include a second finger-ring electrode, wherein the second finger-ring electrode is configured to be housed in a second finger-ring to be worn on a Y-finger of the wearer's second hand, wherein the ECG recording module is configured to comprise a wired or wireless amplifier, wherein the first finger-ring electrode and the second finger-ring electrode are configured as wireless electrodes and are in wireless communication with the ECG recording module, wherein the ECG recording module is configured to record ECG data via at least the first finger-ring electrode and the second finger-ring electrode, and wherein the ECG recording module is in electronic communication with the processing unit that is configure to analyze the ECG data recorded by the ECG recording module and the processing unit is further configured to process the ECG data recorded by the ECG recording module to assess the wearer's ECG profile and to detect presence or cessation of ECG data suggestive of a serious cardiac arrhythmia or an impending serious cardiac arrhythmia.

10. The smart-ring automatic anti-arrhythmia system of claim 8, wherein the first finger-ring electrode is configured to be partially embedded in an inner surface of the first finger-ring with slight protrusion at the inner surface of the first finger-ring, wherein the first finger-ring is configured to comprise elastic, adaptable flexible material and the elastic, adaptable flexible material is configured to have appropriate size, elasticity, adaptability and flexibility such that the first finger-ring electrode is snugly in contact with the X-finger skin of the wearer's first hand when the first finger-ring is worn on the X-finger of the wearer's first hand, wherein the first auricular electrode is configured to be partially embedded in a surface of the first in-the-ear portion of the first auricular housing structure with slight protrusion at the surface of the first in-the-ear portion of the first auricular housing structure, wherein the first in-the-ear portion is configured to comprise elastic, adaptable flexible material and the elastic, adaptable flexible material is configured to have appropriate elasticity, adaptability and flexibility such that the first auricular electrode is snugly in contact with the skin of the external ear canal and skin of a bowl-shaped area of tragus-concha of the wearer's first ear when the in-the-ear portion is inserted into the external ear canal and the bowl-shaped area of tragus-concha of the wearer's first ear, wherein the ear-finger ECG system further comprises a network interface and at least one of the following: a speaker, a vibrator, and a display screen on a client device of the wearer, wherein the network interface is in electronic communication with the processing unit, the speaker, the vibrator, and the display screen on the client device of the wearer, wherein the network interface is configured to generate a visible notification to the display screen on the client device of the wearer describing the wearer's electrocardiographic profile, wherein the network interface is configured to generate an audible notification to the speaker when the processing unit detects presence of a serious cardiac arrhythmia or an impending serious cardiac arrhythmia, and wherein the network interface is further configured to generate a tactile notification to the vibrator when the processing unit detects presence of a serious cardiac arrhythmia or an impending serious cardiac arrhythmia.

11. The smart-ring automatic anti-arrhythmia system of claim 9, wherein the first finger-ring electrode is configured to be partially embedded in an inner surface of the first finger-ring with slight protrusion at the inner surface of the first finger-ring, wherein the first finger-ring is configured to comprise elastic, adaptable and flexible material and the elastic, adaptable flexible material is configured to have appropriate size, elasticity, adaptability and flexibility such that the first finger-ring electrode is snugly in contact with the X-finger skin of the wearer's first hand when the first finger-ring is worn on the X-finger of the wearer's first hand, wherein the second finger-ring electrode is configured to be partially embedded in an inner surface of the second finger-ring with slight protrusion at the inner surface of the second finger-ring, wherein the second finger-ring is configured to comprise elastic, adaptable and flexible material and the elastic, adaptable flexible material is configured to have appropriate size, elasticity, adaptability and flexibility such that the second finger-ring electrode is snugly in contact with the Y-finger skin of the wearer's second hand when the second finger-ring is worn on the Y-finger of the wearer's second hand, wherein the finger-finger ECG system further comprises a network interface and at least one of the following: a speaker, a vibrator, and a display screen on a client device of the wearer, wherein the network interface is in electronic communication with the processing unit, the speaker, the vibrator, and the display screen on the client device of the wearer, wherein the network interface is configured to generate a visible notification to the display screen on the client device of the wearer describing the wearer's electrocardiographic profile, wherein the network interface is configured to generate an audible notification to the speaker when the processing unit detects presence of a serious cardiac arrhythmia or an impending serious cardiac arrhythmia, and wherein the network interface is further configured to generate a tactile notification to the vibrator when the processing unit detects presence of a serious cardiac arrhythmia or an impending serious cardiac arrhythmia.

12. The smart-ring automatic anti-arrhythmia system of claim 7, wherein the smart-ring electrocardiogram (ECG) monitoring system is configured as an ear-toe ECG system, wherein the ear-toe ECG system comprises an ECG recording module, wherein the first ring electrode of the at least two wired or wireless ECG electrodes is configured as a first toe-ring electrode to be housed in a first toe-ring to be worn on an X-toe of a wearer's first foot, wherein the at least two wired or wireless ECG electrodes further include a first auricular electrode, wherein the first auricular electrode is configured to be housed in a first in-the-ear portion of a first auricular housing structure, wherein the first in-the-ear portion of the first auricular housing structure is configured to be placed inside the external ear canal and a bowl-shaped area of tragus-concha of the wearer's first ear when in use, wherein the ECG recording module is configured to comprise a wired or wireless amplifier, wherein the first toe-ring electrode and the first auricular electrode are configured as wireless electrodes, wherein the first toe-ring electrode and the first auricular electrode are in wireless communication with the ECG recording module, wherein the ECG recording module is configured to record ECG data of the wearer via at least the first toe-ring electrode and the first auricular electrode, wherein the processing unit is in electronic communication with the ECG recording module, wherein the processing unit is further configured to analyze the ECG data recorded by the ECG recording module to assess the wearer's ECG profile and to detect presence or cessation of a serious cardiac arrhythmia or an impending serious cardiac arrhythmia, wherein the first toe-ring electrode is configured to be partially embedded in an inner surface of the first toe-ring with slight protrusion at the inner surface of the first toe-ring, wherein the first toe-ring is configured to comprise elastic, adaptable flexible material and the elastic, adaptable flexible material is configured to have appropriate size, elasticity, adaptability and flexibility such that the first toe-ring electrode is snugly in contact with the X-toe skin of the wearer's first foot when the first toe-ring is worn on the X-toe of the wearer's first foot, wherein the first auricular electrode is configured to be partially embedded in a surface of the first in-the-ear portion of the first auricular housing structure with slight protrusion at the surface of the first in-the-ear portion of the first auricular housing structure, and wherein the first in-the-ear portion is configured to comprise elastic, adaptable flexible material and the elastic, adaptable flexible material is configured to have appropriate elasticity, adaptability and flexibility such that the first auricular electrode is snugly in contact with the skin of the external ear canal and skin of a bowl-shaped area of tragus-concha of the wearer's first ear when the in-the-ear portion is placed into the external ear canal and the bowl-shaped area of tragus-concha of the wearer's first ear.

13. The smart-ring automatic anti-arrhythmia system of claim 7, wherein the smart-ring electrocardiogram (ECG) monitoring system is configured as a finger-toe ECG system, wherein the finger-toe ECG system comprises an ECG recording module, wherein the first ring electrode of the at least two wired or wireless ECG electrodes is configured as a first toe-ring electrode to be housed in a first toe-ring to be worn on an X-toe of a wearer's first foot, wherein the at least two wired or wireless ECG electrodes further include a first finger-ring electrode, wherein the first finger-ring electrode is configured to be housed in a first finger-ring to be worn on an X-finger of a first hand of the wearer, wherein the ECG recording module is configured to comprise a wired or wireless amplifier, wherein the first toe-ring electrode and the first finger-ring electrode are configured as wireless electrodes and are in wireless communication with the ECG recording module, wherein the ECG recording module is configured to record ECG data via at least the first toe-ring electrode and the first finger-ring electrode, and wherein the ECG recording module is in electronic communication with the processing unit that is configured to analyze the ECG data recorded by the ECG recording module and the processing unit is further configured to process the ECG data recorded by the ECG recording module to assess the wearer's ECG profile and to detect presence or cessation of ECG data suggestive of a serious cardiac arrhythmia or an impending serious cardiac arrhythmia, wherein the first toe-ring electrode is configured to be partially embedded in an inner surface of the first toe-ring with slight protrusion at the inner surface of the first toe-ring, wherein the first toe-ring is configured to comprise elastic, adaptable and flexible material and the elastic, adaptable flexible material is configured to have appropriate size, elasticity, adaptability and flexibility such that the first toe-ring electrode is snugly in contact with the X-toe skin of the wearer's first foot when the first toe-ring is worn on the X-toe of the wearer's first foot, wherein the first finger-ring electrode is configured to be partially embedded in an inner surface of the first finger-ring with slight protrusion at the inner surface of the first finger-ring, wherein the first finger-ring is configured to comprise elastic, adaptable and flexible material and the elastic, adaptable flexible material is configured to have appropriate size, elasticity, adaptability and flexibility such that the first finger-ring electrode is snugly in contact with the X-finger skin of the wearer's first hand when the first finger-ring is worn on the X-finger of the wearer's first hand.

14. The smart-ring automatic anti-arrhythmia system of claim 7, wherein the smart-ring electrocardiogram (ECG) monitoring system is configured as a toe-toe ECG system, wherein the toe-toe ECG system comprises an ECG recording module, wherein the first ring electrode of the at least two wired or wireless ECG electrodes is configured as a first toe-ring electrode to be housed in a first toe-ring to be worn on an X-toe of a wearer's first foot, wherein the at least two wired or wireless ECG electrodes further include a second toe-ring electrode, wherein the second-toe-ring electrode is configured to be housed in a second toe-ring to be worn on a Y-toe of a second foot of the wearer, wherein the ECG recording module is configured to comprise a wired or wireless amplifier, wherein the first toe-ring electrode and the second toe-ring electrode are configured as wireless electrodes and are in wireless communication with the ECG recording module, wherein the ECG recording module is configured to record ECG data via at least the first toe-ring electrode and the second toe-ring electrode, and wherein the ECG recording module is in electronic communication with the processing unit that is configured to analyze the ECG data recorded by the ECG recording module and the processing unit is further configured to process the ECG data recorded by the ECG recording module to assess the wearer's ECG profile and to detect presence or cessation of ECG data suggestive of a serious cardiac arrhythmia or an impending serious cardiac arrhythmia, wherein the first toe-ring electrode is configured to be partially embedded in an inner surface of the first toe-ring with slight protrusion at the inner surface of the first toe-ring, wherein the first toe-ring is configured to comprise elastic, adaptable and flexible material and the elastic, adaptable flexible material is configured to have appropriate size, elasticity, adaptability and flexibility such that the first toe-ring electrode is snugly in contact with the X-toe skin of the wearer's first foot when the first toe-ring is worn on the X-toe of the wearer's first foot, wherein the second-toe-ring electrode is configured to be partially embedded in an inner surface of the second toe-ring with slight protrusion at the inner surface of the second toe-ring, wherein the second toe-ring is configured to comprise elastic, adaptable and flexible material and the elastic, adaptable flexible material is configured to have appropriate size, elasticity, adaptability and flexibility such that the second toe-ring electrode is snugly in contact with the Y-toe skin of the wearer's second foot when the second toe-ring is worn on the Y-toe of the wearer's second foot.

15. The smart-ring automatic anti-arrhythmia system of claim 7, further comprising a second transcutaneous auricular vagus nerve stimulation unit (second taVNS unit), wherein the second taVNS unit comprises a second stimulating electrode to be coupled to the wearer's second ear when in use, wherein the second stimulating electrode is configured to contact vagus innervated auricular skin of the wearer's second ear, wherein the vagus innervated auricular skin that the second stimulating electrode is configured to contact is selected from at least one of the following: external ear canal of the wearer's second ear, tragus of the wearer's second ear, cymba concha of the wearer's second ear and cavum concha of the wearer's second ear, wherein the second taVNS unit is in electronic communication with the processing unit, wherein when presence of ECG signals suggestive of at least one serious cardiac arrhythmia is detected by the processing unit, the processing unit is further configured to immediately send arrhythmia-warning-signals to the second taVNS unit and the second taVNS unit is configured to automatically start sending pre-determined electric stimuli to the vagus innervated auricular skin of the wearer's second ear to which the second stimulating electrode is in contact with, and wherein when cessation of ECG signals suggestive of the at least one serious cardiac arrhythmia is detected by the processing unit, the processing unit is configured to immediately send arrhythmia-cessation-signals to the second taVNS unit and the second taVNS unit is configured to automatically stop the electric stimuli to the vagus innervated auricular skin of the wearer's second ear to which the second stimulating electrode is in contact with, wherein when presence of ECG signals suggestive of at least one impending serious cardiac arrhythmia is detected by the processing unit, the processing unit is configured to immediately send impending-arrhythmia-warning-signals to the second taVNS unit and the second taVNS unit is configured to automatically start sending pre-determined electric stimuli to the vagus innervated auricular skin of the wearer's second ear to which the second stimulating electrode is in contact with, wherein when cessation of ECG signals suggestive of the at least one impending serious cardiac arrhythmia is detected by the processing unit, the processing unit is configured to immediately send impending-arrhythmia-cessation-signals to the second taVNS unit and the second taVNS unit is configured to automatically stop the electric stimuli to the vagus innervated auricular skin of the wearer's second ear to which the second stimulating electrode is in contact with, wherein the first stimulating electrode of the first taVNS unit is configured to be housed in a first in-the-ear portion of a first auricular housing structure, wherein the second stimulating electrode is configured to be housed in a second in-the-ear portion of a second auricular housing structure, wherein the first stimulating electrode is configured to be partially embedded in a surface of the first in-the-ear portion of the first auricular housing structure with slight protrusion at the surface of the first in-the-ear portion of the first auricular housing structure, wherein the first in-the-ear portion is configured to comprise elastic, adaptable flexible material and the elastic, adaptable flexible material is configured to have appropriate elasticity, adaptability and flexibility such that the first stimulating electrode is snugly in contact with the skin of the external ear canal and skin of a bowl-shaped area of tragus-concha of the wearer's first ear when the in-the-ear portion is inserted into the external ear canal and the bowl-shaped area of tragus-concha of the wearer's first ear, wherein the second stimulating electrode is configured to be partially embedded in a surface of the second in-the-ear portion of the second auricular housing structure with slight protrusion at the surface of the second in-the-ear portion of the second auricular housing structure, and wherein the second in-the-ear portion is configured to comprise elastic, adaptable flexible material and the elastic, adaptable flexible material is configured to have appropriate elasticity, adaptability and flexibility such that the second stimulating electrode is snugly in contact with the skin of the external ear canal and skin of a bowl-shaped area of tragus-concha of the wearer's second ear when the second in-the-ear portion is inserted into the external ear canal and the bowl-shaped area of tragus-concha of the wearer's second ear.

16. (canceled)

17. The wearable self-installable self-removable electrocardiogram (ECG) monitoring system of claim 16, wherein the wearable self-installable self-removable ECG monitoring system is configured as an ear-finger ECG system, wherein the first self-installable-housing structure is configured as a first finger-ring, wherein the second self-installable-housing structure is configured as a first in-the-ear portion of a first auricular housing structure, wherein the at least two wired or wireless self-installable ECG electrodes includes a first finger-ring electrode and a first auricular electrode, wherein the first finger-ring electrode of the at least two wired or wireless self-installable ECG electrodes is configured to be housed in the first finger-ring to be worn on an X-finger of the first hand of the wearer, wherein the first auricular electrode is configured to be housed in the first in-the-ear portion of the first auricular housing structure, wherein the first in-the-ear portion is configured to be placed into an external ear canal and a bowl-shaped area of tragus-concha of a first ear of the wearer when in use, wherein the ECG recording module is in electronic communication with the first finger-ring electrode and the first auricular electrode of the at least two wired or wireless self-installable ECG electrodes, wherein the ECG recording module is configured to comprise a wired or wireless amplifier, wherein the first finger-ring electrode and the first auricular electrode are configured as wireless electrodes, wherein the first finger-ring electrode and the first auricular electrode are in wireless communication with the ECG recording module, wherein the ECG recording module is configured to record ECG data of the wearer via at least the first finger-ring electrode and the first auricular electrode, wherein the processing unit is in electronic communication with the ECG recording module, wherein the processing unit is configured to analyze the ECG data recorded by the ECG recording module to detect presence or cessation of at least one serious cardiac arrhythmia, and wherein the processing unit is further configured to analyze the ECG data recorded by the ECG recording module to detect presence or cessation of at least one impending serious cardiac arrhythmia.

18. The wearable self-installable self-removable electrocardiogram (ECG) monitoring system of claim 16, wherein the wearable self-installable self-removable ECG monitoring system is configured as a finger-finger ECG system, wherein the first self-installable-housing structure is configured as a first finger-ring, wherein the second self-installable-housing structure is configured as a second finger-ring, wherein the at least two wired or wireless self-installable ECG electrodes includes a first finger-ring electrode and a second finger-ring electrode, wherein the first finger-ring electrode of the at least two wired or wireless self-installable ECG electrodes is configured to be housed in the first finger-ring to be worn on an X-finger of the first hand of the wearer, wherein the second finger-ring electrode of the at least two wired or wireless self-installable ECG electrodes is configured to be housed in the second finger-ring to be worn on a Y-finger of a second hand of the wearer, wherein the ECG recording module is in electronic communication with the first finger-ring electrode and the second finger-ring electrode of the at least two wired or wireless self-installable ECG electrodes, wherein the ECG recording module is configured to comprise a wired or wireless amplifier, wherein the first finger-ring electrode and the second finger-ring electrode are configured as wireless electrodes and are in wireless communication with the ECG recording module, wherein the ECG recording module is configured to record ECG data via at least the first finger-ring electrode and the second finger-ring electrode, and wherein the ECG recording module is in electronic communication with the processing unit that is configured to analyze the ECG data recorded by the ECG recording module and the processing unit is configured to process the ECG data recorded by the ECG recording module to detect presence or cessation of ECG data suggestive of at least one serious cardiac arrhythmia, and wherein the processing unit is further configured to process the ECG data recorded by the ECG recording module to detect presence or cessation of ECG data suggestive of at least one impending serious cardiac arrhythmia.

19. The wearable self-installable self-removable electrocardiogram (ECG) monitoring system configured as the ear-finger ECG system of claim 17, wherein the first finger-ring electrode is configured to be partially embedded in an inner surface of the first finger-ring with slight protrusion at the inner surface of the first finger-ring, wherein the first finger-ring is configured to comprise elastic, adaptable flexible material and the elastic, adaptable flexible material is configured to have appropriate elasticity, adaptability and flexibility such that the first finger-ring electrode is snugly in contact with the X-finger skin of the wearer's first hand when the first ring is worn on the X-finger of the wearer's first hand, wherein the first auricular electrode is configured to be partially embedded in a surface of the first in-the-ear portion of the first auricular housing structure with slight protrusion at the surface of the first in-the-ear portion of the first auricular housing structure, wherein the first in-the-ear portion of the first auricular housing structure is configured to comprise elastic, adaptable flexible material and the elastic, adaptable flexible material is configured to have appropriate elasticity, adaptability and flexibility such that the first in-the-ear portion is snugly in contact with the skin of the external ear canal and skin of the bowl-shaped area of tragus-concha of the wearer's first ear when the first in-the-ear portion is placed into the external ear canal and the bowl-shaped area of tragus-concha of the wearer's first ear, wherein the ear-finger ECG system further comprises a network interface and at least one of the following: a speaker, a vibrator, and a display screen on a client device of the wearer, wherein the network interface is in electronic communication with the processing unit, the speaker, the vibrator, and the display screen on the client device of the wearer, wherein the network interface is configured to generate a visible notification to the display screen on the client device of the wearer describing the wearer's electrocardiographic profile, wherein the network interface is configured to generate an audible notification to the speaker when the processing unit detects presence of a serious cardiac arrhythmia or an impending serious cardiac arrhythmia, and wherein the network interface is further configured to generate a tactile notification to the vibrator when the processing unit detects presence of a serious cardiac arrhythmia or an impending serious cardiac arrhythmia.

20. The wearable self-installable self-removable electrocardiogram (ECG) monitoring system configured as the finger-finger ECG system of claim 18, wherein the first finger-ring electrode is configured to be partially embedded in an inner surface of the first finger-ring with slight protrusion at the inner surface of the first finger-ring, wherein the first finger-ring is configured to comprise elastic, adaptable flexible material and the elastic, adaptable flexible material is configured to have appropriate size, elasticity, adaptability and flexibility such that the first finger-ring electrode is snugly in contact with the X-finger skin of the wearer's first hand when the first finger-ring is worn on the X-finger of the wearer's first hand, wherein the second finger-ring electrode is configured to be partially embedded in an inner surface of the second finger-ring with slight protrusion at the inner surface of the second finger-ring, wherein the second finger-ring is configured to comprise elastic, adaptable flexible material and the elastic, adaptable flexible material is configured to have appropriate size, elasticity, adaptability and flexibility such that the second finger-ring electrode is snugly in contact with the Y-finger skin of the wearer's second hand when the second finger-ring is worn on the Y-finger of the wearer's second hand, wherein the finger-finger ECG system further comprises a network interface and at least one of the following: a speaker, a vibrator, and a display screen on a client device of the wearer, wherein the network interface is in electronic communication with the processing unit, the speaker, the vibrator, and the display screen on the client device of the wearer, wherein the network interface is configured to generate a visible notification to the display screen on the client device of the wearer describing the wearer's electrocardiographic profile, wherein the network interface is configured to generate an audible notification to the speaker when the processing unit detects presence of a serious cardiac arrhythmia or an impending serious cardiac arrhythmia, and wherein the network interface is further configured to generate a tactile notification to the vibrator when the processing unit detects presence of a serious cardiac arrhythmia or an impending serious cardiac arrhythmia.

21. The wearable self-installable self-removable electrocardiogram (ECG) monitoring system of claim 16, wherein the wearable self-installable self-removable electrocardiogram (ECG) monitoring system is configured as an ear-toe ECG system, wherein the ear-toe ECG system comprises an ECG recording module, wherein the first self-installable-housing structure is configured as a first toe-ring, wherein the second self-installable-housing structure is configured as a first in-the-ear portion of a first auricular housing structure, wherein the at least two wired or wireless self-installable ECG electrodes includes a first toe-ring electrode and a first auricular electrode, wherein the first toe-ring electrode of the at least two wired or wireless self-installable ECG electrodes is configured to be housed in the first toe-ring to be worn on an X-toe of the first foot of the wearer, wherein the first auricular electrode is configured to be housed in the first in-the-ear portion of the first auricular housing structure, wherein the first in-the-ear portion is configured to be placed into an external ear canal and a bowl-shaped area of tragus-concha of a first ear of the wearer when in use, wherein the ECG recording module is configured to comprise a wired or wireless amplifier, wherein the first toe-ring electrode and the first auricular electrode are configured as wireless electrodes, wherein the first toe-ring electrode and the first auricular electrode are in wireless communication with the ECG recording module, wherein the ECG recording module is configured to record ECG data of the wearer via at least the first toe-ring electrode and the first auricular electrode, wherein the processing unit is in electronic communication with the ECG recording module, wherein the processing unit is configured to analyze the ECG data recorded by the ECG recording module to assess the wearer's ECG profile and to detect presence or cessation of at least one serious cardiac arrhythmia or at least one impending serious cardiac arrhythmia, wherein the first toe-ring electrode is configured to be partially embedded in an inner surface of the first toe-ring with slight protrusion at the inner surface of the first toe-ring, wherein the first toe-ring is configured to comprise elastic, adaptable flexible material and the elastic, adaptable flexible material is configured to have appropriate size, elasticity, adaptability and flexibility such that the first toe-ring electrode is snugly in contact with the X-toe skin of the wearer's first foot when the first toe-ring is worn on the X-toe of the wearer's first foot, wherein the first auricular electrode is configured to be partially embedded in a surface of the first in-the-ear portion of the first auricular housing structure with slight protrusion at the surface of the first in-the-ear portion of the first auricular housing structure, and wherein the first in-the-ear portion is configured to comprise elastic, adaptable flexible material and the elastic, adaptable flexible material is configured to have appropriate elasticity, adaptability and flexibility such that the first auricular electrode is snugly in contact with the skin of the external ear canal and skin of a bowl-shaped area of tragus-concha of the wearer's first ear when the in-the-ear portion is inserted into the external ear canal and the bowl-shaped area of tragus-concha of the wearer's first ear.

22. The wearable self-installable self-removable electrocardiogram (ECG) monitoring system of claim 16, wherein the wearable self-installable self-removable electrocardiogram (ECG) monitoring system is configured as a finger-toe ECG system, wherein the finger-toe ECG system comprises an ECG recording module, wherein the first self-installable-housing structure is configured as a first toe-ring, wherein the second self-installable-housing structure is configured as a first finger-ring, wherein the at least two wired or wireless self-installable ECG electrodes includes a first toe-ring electrode and a first finger-ring electrode, wherein the first toe-ring electrode of the at least two wired or wireless self-installable ECG electrodes is configured to be housed in the first toe-ring to be worn on an X-toe of the first foot of the wearer, wherein the first finger-ring electrode is configured to be housed in the first finger-ring to be worn on an X-finger of the wearer's first hand, wherein the ECG recording module is configured to comprise a wired or wireless amplifier, wherein the first toe-ring electrode and the first finger-ring electrode are configured as wireless electrodes and are in wireless communication with the ECG recording module, wherein the ECG recording module is configured to record ECG data via at least the first toe-ring electrode and the first finger-ring electrode, and wherein the ECG recording module is in electronic communication with the processing unit that is configure to analyze the ECG data recorded by the ECG recording module and the processing unit is configured to process the ECG data recorded by the ECG recording module to assess the wearer's ECG profile and to detect presence or cessation of ECG data suggestive of at least one serious cardiac arrhythmia or at least one impending serious cardiac arrhythmia, wherein the first toe-ring electrode is configured to be partially embedded in an inner surface of the first toe-ring with slight protrusion at the inner surface of the first toe-ring, wherein the first toe-ring is configured to comprise elastic, adaptable flexible material and the elastic, adaptable flexible material is configured to have appropriate size, elasticity, adaptability and flexibility such that the first toe-ring electrode is snugly in contact with the X-toe skin of the wearer's first foot when the first toe-ring is worn on the X-toe of the wearer's first foot, wherein the first finger-ring electrode is configured to be partially embedded in an inner surface of the first finger-ring with slight protrusion at the inner surface of the first finger-ring, wherein the first finger-ring is configured to comprise elastic, adaptable flexible material and the elastic, adaptable flexible material is configured to have appropriate size, elasticity, adaptability and flexibility such that the first finger-ring electrode is snugly in contact with the X-finger skin of the wearer's first hand when the first finger-ring is worn on the X-finger of the wearer's first hand.

23. The wearable self-installable self-removable electrocardiogram (ECG) monitoring system of claim 16, wherein the wearable self-installable self-removable electrocardiogram (ECG) monitoring system is configured as a toe-toe ECG system, wherein the toe-toe ECG system comprises an ECG recording module, wherein the first self-installable-housing structure is configured as a first toe-ring, wherein the second self-installable-housing structure is configured as a second toe-ring, wherein the at least two wired or wireless self-installable ECG electrodes includes a first toe-ring electrode and a second toe-ring electrode, wherein the first toe-ring electrode of the at least two wired or wireless self-installable ECG electrodes is configured to be housed in the first toe-ring to be worn on an X-toe of the first foot of the wearer, wherein the second toe-ring electrode is configured to be housed in the second toe-ring to be worn on an Y-toe of the wearer's second foot, wherein the ECG recording module is configured to comprise a wired or wireless amplifier, wherein the first toe-ring electrode and the second toe-ring electrode are configured as wireless electrodes and are in wireless communication with the ECG recording module, wherein the ECG recording module is configured to record ECG data via at least the first toe-ring electrode and the second toe-ring electrode, and wherein the ECG recording module is in electronic communication with the processing unit that is configure to analyze the ECG data recorded by the ECG recording module and the processing unit is configured to process the ECG data recorded by the ECG recording module to assess the wearer's ECG profile and to detect presence or cessation of ECG data suggestive of at least one serious cardiac arrhythmia or at least one impending serious cardiac arrhythmia, wherein the first toe-ring electrode is configured to be partially embedded in an inner surface of the first toe-ring with slight protrusion at the inner surface of the first toe-ring, wherein the first toe-ring is configured to comprise elastic, adaptable flexible material and the elastic, adaptable flexible material is configured to have appropriate size, elasticity, adaptability and flexibility such that the first toe-ring electrode is snugly in contact with the X-toe skin of the wearer's first foot when the first toe-ring is worn on the X-toe of the wearer's first foot, wherein the second toe-ring electrode is configured to be partially embedded in an inner surface of the second toe-ring with slight protrusion at the inner surface of the second toe-ring, wherein the second toe-ring is configured to comprise elastic, adaptable flexible material and the elastic, adaptable flexible material is configured to have appropriate size, elasticity, adaptability and flexibility such that the second toe-ring electrode is snugly in contact with the Y-toe skin of the wearer's second foot when the second toe-ring is worn on the Y-toe of the wearer's second foot.

24. A chest-arm automatic anti-arrhythmia system, comprising:
a chest-arm electrocardiogram (ECG) monitoring system, comprising an ECG recording module, wherein the ECG recording module comprises at least two wired or wireless ECG electrodes configured to contact separate locations of a wearer's skin, wherein the at least two wired or wireless ECG electrodes are selected from one of: chest-contacting ECG electrodes and arm-contacting ECG electrodes, wherein the at least two separate areas of the wearer's skin is selected from one of the following: the wearer's chest and an arm of the wearer, wherein the ECG recording module is in electronic communication with each ECG electrode of the at least two wired or wireless ECG electrodes, and wherein the ECG recording module is configured to record ECG data of the wearer via the at least two wired or wireless ECG electrodes;
a first transcutaneous auricular vagus nerve stimulation unit (first taVNS unit), the first taVNS unit having a first stimulating electrode configured to contact vagus innervated auricular skin of the wearer's first ear, and wherein the vagus innervated auricular skin that the first stimulating electrode is configured to contact is selected from at least one of the following: external ear canal of the wearer's first ear, tragus of the wearer's first ear, cymba concha of the wearer's first ear and cavum concha of the wearer's first ear; and
a processing unit in electronic communication with the chest-arm ECG monitoring system and the first taVNS unit, wherein the processing unit is configured to analyze the ECG data recorded by the chest-arm ECG monitoring system to assess the wearer's ECG profile and to detect the presence or cessation of ECG signals suggestive of at least one serious cardiac arrhythmia or at least one impending serious cardiac arrhythmia; wherein when presence of ECG signals suggestive of at least one serious cardiac arrhythmia is detected by the processing unit, the processing unit is configured to immediately send arrhythmia-warning-signals to the first taVNS unit and upon receiving the arrhythmia-warning-signals from the processing unit the first taVNS unit is configured to automatically start sending pre-determined electric stimuli to the vagus innervated auricular skin of the wearer's first ear to which the first stimulating electrode is in contact with; wherein when presence of ECG signals suggestive of at least one impending serious cardiac arrhythmia is detected by the processing unit, the processing unit is configured to immediately send impending-arrhythmia-warning-signals to the first taVNS unit and upon receiving the impending-arrhythmia-warning-signals from the processing unit the first taVNS unit is configured to automatically start sending pre-determined electric stimuli to the vagus innervated auricular skin of the wearer's first ear to which the first stimulating electrode is in contact with; wherein when cessation of ECG signals suggestive of the at least one serious cardiac arrhythmia is detected by the processing unit, the processing unit is configured to immediately send arrhythmia-cessation-signals to the first taVNS unit and upon receiving the arrhythmia-cessation-signals from the processing unit the first taVNS unit is configured to automatically stop sending pre-determined electric stimuli to the vagus innervated auricular skin of the wearer's first ear to which the first stimulating electrode is in contact with, and wherein when cessation of ECG signals suggestive of the at least one impending serious cardiac arrhythmia is detected by the processing unit, the processing unit is further configured to immediately send impending-arrhythmia-cessation signals to the first taVNS unit and upon receiving the impending-arrhythmia-cessation-signals from the processing unit the first taVNS unit is configured to automatically stop sending pre-determined electric stimuli to the vagus innervated auricular skin of the wearer's first ear to which the first stimulating electrode is in contact with.

25. An automatic anti-arrhythmia system comprising:
an electrocardiogram (ECG) monitoring system, comprising an ECG recording module, wherein the ECG recording module comprises at least two wired or wireless ECG electrodes configured to contact separate locations of a wearer's skin, and wherein the ECG recording module is configured to record ECG data of the wearer via the at least two wired or wireless ECG electrodes;
a first transcutaneous auricular vagus nerve stimulation unit (first taVNS unit), comprising a first stimulating electrode, wherein the first stimulating electrode of the first taVNS unit is configured to be housed in a first housing structure, wherein at least part of the first housing structure is configured to be inserted into at least one of the following: an external ear canal and a bowl-shaped area of tragus-concha of a first ear of a wearer when in use, and wherein the first stimulating electrode of the first taVNS unit is configured to be naturally snugly in contact with one of the following: the skin of the external ear canal and the skin of the bowl-shaped area of tragus-concha of the wearer's first ear when the at least part of the first housing structure is inserted into at least one of the external ear canal and the bowl-shaped area of tragus-concha of the wearer's first ear such that the first stimulating electrode of the first taVNS unit is configured to be naturally snugly in contact with vagus innervated auricular skin when the at least part of the first housing structure is inserted into at least one of the external ear canal and the bowl-shaped area of tragus-concha of the wearer's first ear; and
a processing unit in electronic communication with the ECG monitoring system and the first taVNS unit,
i. wherein the processing unit is configured to analyze the ECG data of the wearer recorded by the ECG monitoring system to assess the wearer's ECG profile and to detect the presence or cessation of ECG data suggestive of at least one serious cardiac arrhythmia, wherein the processing unit is further configured to analyze the ECG data recorded by the ECG monitoring system to assess the wearer's ECG profile and to detect the presence or cessation of ECG signals suggestive of at least one impending serious cardiac arrhythmia;
ii. wherein when presence of ECG data suggestive of at least one serious cardiac arrhythmia is detected by the processing unit, the processing unit is configured to immediately send arrhythmia-warning-signals to the first taVNS unit and upon receiving the arrhythmia-warning-signals from the processing unit the first taVNS unit is configured to automatically start sending pre-determined electric stimuli to the vagus innervated auricular skin of the wearer's first ear to which the first stimulating electrode is in contact with;
iii. wherein when cessation of ECG signals suggestive of the at least one serious cardiac arrhythmia is detected by the processing unit, the processing unit is further configured to immediately send arrhythmia-cessation-signals to the first taVNS unit and upon receiving arrhythmia-cessation signals from the processing unit the first taVNS unit is configured to automatically stop sending pre-determined electric stimuli to the vagus innervated auricular skin of the wearer's first ear to which the first stimulating electrode is in contact with;
iv. wherein when presence of ECG data suggestive of at least one impending serious cardiac arrhythmia is detected by the processing unit, the processing unit is configured to immediately send impending-arrhythmia-warning-signals to the first taVNS unit and upon receiving the impending-arrhythmia-warning-signals from the processing unit the first taVNS unit is configured to automatically start sending pre-determined electric stimuli to the vagus innervated auricular skin of the wearer's first ear to which the first stimulating electrode is in contact with; and
v. wherein when cessation of ECG signals suggestive of the at least one impending serious cardiac arrhythmia is detected by the processing unit, the processing unit is further configured to immediately send impending-arrhythmia-cessation-signals to the first taVNS unit and upon receiving impending-arrhythmia-cessation signals from the processing unit the first taVNS unit is configured to automatically stop sending pre-determined electric stimuli to the vagus innervated auricular skin of the wearer's first ear to which the first stimulating electrode is in contact with.
